(19) United States
(12) Patent Application Publication
(10) Pub. No.: US 2023/0399688 A1
(43) Pub. Date: Dec. 14, 2023

(54) COMPOSITIONS AND MULTIPLEXED SYSTEMS FOR COUPLED CELL-FREE TRANSCRIPTION-TRANSLATION AND PROTEIN SYNTHESIS AND METHODS FOR USING THEM

(71) Applicant: GENOMATICA, INC., San Diego, CA (US)

(72) Inventors: Stephanie CULLER, San Diego, CA (US); Ihsiung Brandon CHEN, San Diego, CA (US); Priti PHARKYA, San Diego, CA (US); Steve VAN DIEN, San Diego, CA (US); Nelson BARTON, San Diego, CA (US)

(21) Appl. No.: 18/120,951

(22) Filed: Mar. 13, 2023

Related U.S. Application Data
(63) Continuation of application No. 15/752,884, filed on Feb. 14, 2018, now abandoned.
(60) Provisional application No. 62/207,844, filed on Aug. 20, 2015.

(51) Int. Cl. C12Q 1/6869; A61K 35/66; C12P 21/02; C12P 19/34; A61K 36/02; A61K 36/06; C12N 5/16; C12N 15/09; C40B 40/04

(52) U.S. Cl. CPC C12Q 1/6869; A61K 35/66; C12P 21/02; C12P 19/34; A61K 36/02; A61K 36/06; C12N 5/16; C12N 15/09; C40B 40/04; Y02A 50/30; A61K 2236/15; A61K 2236/30; C12N 2330/50

(57) ABSTRACT

In alternative embodiments, provided herein are transcription/translation (TX-TL) systems and methods of using them for use as rapid prototyping platforms for the synthesis, modification and identification of natural products (NPs), and natural product analogs (NPAs) and secondary metabolites, from biosynthetic gene cluster pipelines. In alternative embodiments, exemplary TX-TL systems as provided herein are used for the combinatorial biosynthesis of natural products (NPs), natural product analogs (NPAs) and secondary metabolites. In alternative embodiments, exemplary TX-TL systems as provided herein are used for the rapid prototyping of complex biosynthetic pathways as a way to rapidly assess combinatorial and biosynthetic designs before moving to cellular hosts. In alternative embodiments, these exemplary TX-TL systems are multiplexed for high-throughput (HT) automation and for prototyping engineered platforms for the synthesis or modification of natural products (NPs), and natural product analogs (NPAs) and secondary metabolites analogs.

### RELATED APPLICATIONS

This application is a continuation of U.S. National Phase application U.S. Ser. No. 15/752,884, filed Feb. 14, 2018, now pending, which is a national phase application claiming benefit of priority under 35 U.S.C. § 371 to Patent Convention Treaty (PCT) International Application serial number PCT/US2016/047704, filed Aug. 19, 2016, now expired, which claims the benefit of priority to U.S. Provisional Patent Application Serial No. (USSN) 62/207,844, filed Aug. 20, 2015, now expired. The aforementioned applications are expressly incorporated herein by reference in their entirety and for all purposes.

### TECHNICAL FIELD

This invention generally relates to genetic and cell engineering and biosynthetic processes for the production of organic chemicals and natural products, and particularly to in vitro systems for rapid creation of improved production host strains and libraries of new products including natural product analogs.

### BACKGROUND

Research, development and production of useful compounds, including industrial, pharmaceutical and nutraceutical compounds, both natural and synthetic, is very time, labor and cost intensive. False positives arise that require extensive time, labor and cost to exclude. Difficulties exist in creating a diversity of new compounds, especially by chemical means. Historically in vivo approaches have been required to both identify, synthesize and screen new compounds. A limited number of elite production host strains are available, which can be a challenge when developing production of new compounds, particularly complex compounds. There exists a need for methods and systems that facilitate research, development and production of new compounds, especially complex compounds.

### SUMMARY

In alternative embodiments, provided herein are coupled transcription/translation (TX-TL) systems and methods of using them as rapid prototyping platforms for the synthesis, modification and identification of natural products (NPs), and natural product analogs (NPAs) and secondary metabolites, from biosynthetic gene cluster pipelines. In alternative embodiments, exemplary TX-TL (TX/TL) systems as provided herein are used for the combinatorial biosynthesis of natural products (NPs), natural product analogs (NPAs) and secondary metabolites, and libraries of NPs, NPAs and secondary metabolite analogs. In alternative embodiments, exemplary TX-TL systems as provided herein are used for the rapid prototyping of complex biosynthetic pathways (e.g., for making NPs, NPAs and secondary metabolite analogs), e.g., as a way to rapidly assess combinatorial and biosynthetic designs before moving to cellular hosts. In alternative embodiments, these exemplary TX-TL systems are multiplexed for high-throughput (HT) automation, thus, provided are TX-TL HT platforms for rapid prototyping and modification of natural product (NP) gene clusters and the natural products (NPs) they encode and synthesize, and for prototyping engineered platforms for the synthesis or modification of natural products (NPs), and natural product analogs (NPAs) and secondary metabolites analogs. Accordingly, in alternative embodiments, provided herein are methods and systems that facilitate research, development and production of new compounds, especially complex compounds.

In alternative embodiments, provided are products of manufacture comprising a mixture of: at least two (e.g., 2, 3, 4, 5, 6 or more) cytoplasmic extracts; at least two (e.g., 2, 3, 4, 5, 6 or more) nuclear extracts; or at least one cytoplasmic and one nuclear extract, from at least two (e.g., 2, 3, 4, 5, 6 or more) different cells, wherein optionally the mixture is capable of in vitro transcription, translation and/or coupled transcription and translation. In alternative embodiments, the at least two different cells are from different kingdoms, phyla, classes, orders, families, genera or species. The at least two (e.g., 2, 3, 4, 5, 6 or more) different cell extracts can comprise at least one extract from: a prokaryotic or a eukaryotic cell; or, a bacterial cell, a fungal cell, an algae cell, an Archaeal cell, a yeast cell, an insect cell, a plant cell, a mammalian cell or a human cell.

In alternative embodiments of the products of manufacture the mixture: (a) comprises an undiluted liquid isolate from at least one of the at least two (e.g., 2, 3, 4, 5, 6 or more) different cells; (b) comprises a diluted liquid preparation from at least one of the at least two different cells, wherein optionally the cytoplasmic or nuclear extract or combined cytoplasmic and nuclear extract is diluted with a saline or a buffer; (c) comprises an undiluted liquid preparation from at least one of the at least two different cells; or, (d) comprises a lyophilized or equivalent preparation of the mixture of at least two cytoplasmic or nuclear extracts or combined cytoplasmic and nuclear extract from at least two different cells. In alternative embodiments, between about 50% and 99.9%, or about 50%, 51%, 52%, 53%, 54%, 55%, 56%5, 57% 58%5, 59% 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 99.5% of the liquid volume of the extract is from one of the two extracts (from one of the cytoplasmic or nuclear or combined cytoplasmic and nuclear extracts). The mixture of at least two cytoplasmic or nuclear or combined cytoplasmic and nuclear extracts can comprise: at least one extract from a bacterial cell and at least one extract from a eukaryotic cell; at least one extract from a prokaryotic cell and at least one extract from a mammalian cell; at least one extract from a bacterial cell and at least one extract from an insect, a plant, a fungal or a yeast cell; or, extracts from at least two different bacterial cells, two different fungal cells; two different yeast cells, two different insect cells, two different plant cells or two different mammalian cells. The mixture of at least two cytoplasmic or nuclear or combined cytoplasmic and nuclear extracts can comprise: a mixture of a cytoplasmic and a nuclear extract; a mixture of two different cytoplasmic extracts; or a mixture of at least two different nuclear extracts.

In alternative embodiments of the products of manufacture, at least one of the cytoplasmic or nuclear or combined cytoplasmic and nuclear extracts comprises an extract from or an extract derived from: a prokaryote (optionally a bacteria, an Archaea), a eukaryote (optionally a fungi, a plant, an animal, a human); a bacterial isolate from an environmental source or sample; a Saccharomyces cerevisiae or a yeast a Aspergillus or fungus, optionally A. oryzae, A. nidulans; a plant or plant product, optionally a wheat germ, P. somniferum S. lycopersicon, M esculenta, L. japonicas, A. thaliana, Zea mays, Avena spp.; an Escherichia or a Escherichia coli (E. coli); an Actinomyces or a Streptomyces or an Actinobacteria, a Micromonospora; an Ascomycota, Basidiomycota, or a Saccharomycetales; a Penicillium or a Trichocomaceae; a Spodoptera, a Spodoptera frugiperda, a Trichoplusia or a Trichoplusia ni; a Poaceae, a Triticum; an insect cell, optionally Sf9; a rabbit reticulocyte, Chinese hamster ovary (CHO), Human embryonic kidney (HEK) or a HeLa cell, a cultured human-derived cell; or, Leishmania tarentolae, Myxobacteria, Phellinus, Ceratocystis virescens, Cronartium fusiforme, Paenibacillus polymyxa, Mycolatopsis rifamycinica, Clostridium botulinum, Streptomyces verticillus, Marine bacteria, Archaea, Thermococcus S557, Methanocaldococcus jannaschii, Penicillium chrysogenum, Cephalosporium acremonium, Pleurotus ostreatus, Tolypocladium inflatum, Claviceps spp., Aspergillus alliaceus, Taxus brevifolia, Cephalotaxus harringtonii, Artemisia annua, Galanthus spp., Conus magus, Conus magus, Ecteinascidia turbinate, Discodermia dissoluta, Erythropodium caribaeorum or Bugula neritina.

In alternative embodiments of the products of manufacture at least one of the cytoplasmic extracts comprises an extract from or comprises an extract derived from an E. coli; and, at least one of the cytoplasmic extracts comprises an extract from or comprises an extract derived from an Actinomyces or a Streptomyces, and optionally the Actinomyces is: an Amycolatopsis, a Saccharopolyspora, a Streptomyces, Micromonospora; and optionally the Streptomyces is: S. coelicolor, S. albus, S. albus J1074, S. ambofaciens, S. ambofaciens BES2074, S. avermitilis, S. avermitilis SUKA17, S. coelicolor M1154, S. fradiae, S. roseosporus, S. toyocaensis, S. venezuelae, S. cinnamonensis, Streptomyces rapamycinicus, Streptomyces griseus, Streptomyces platensis, Streptomyces spheroides, Streptomyces rimosus, Streptomyces roseosporus and Streptomyces lividans; and optionally the Amycolatopsis is Amycolatopsis mediterranei, Amycolatopsis orientalis; and optionally the Saccharopolyspora is Saccharopolyspora erythraea, Saccharopolyspora spinosa.

In alternative embodiments of the products of manufacture, the cells from which the at least one cytoplasmic or nuclear extract has been derived, before isolation or harvesting of the extract, is: an activated or a stimulated cell; a cell exposed to chemical or a reagent in vitro; a genetically altered cell; a “strain engineered” cell (modification of cells from which the at least one cytoplasmic or nuclear extract has been derived); or, a cultured cell, wherein optionally the cells from which the at least one cytoplasmic or nuclear extract has been derived have had components of central metabolism (for example glycolysis, pentose phosphate pathway, TCA cycle and amino acid biosynthesis), lipid or fatty acid biosynthesis, oxidative phosphorylation and/or protein synthesis upregulated, activated or co-activated, or de-activated, and optionally the cells from which the at least one cytoplasmic or nuclear extract has been derived are cultured under different environmental or in vitro culture conditions (optionally to turn on/off native enzymes, natural products (secondary metabolites) (NP) (including polyketides of class I, II or III, a non-ribosomal peptide or a hybrid polyketide-non ribosomal peptide), a natural product analog (NPA), or proteins; or for the extracts to have greater or fewer amounts of co-factors); and optionally the cells from which the at least one cytoplasmic or nuclear extract has been derived are in mid-log phase, interphase, mitotic (M) phase or cytokinesis, or undergoing mitosis, and optionally the strain engineering comprises ribosome and/or RNA polymerase engineering, optionally comprising: adding or making rpoB and rpsL mutants (components of ribosomal subunits) to enhance secondary metabolite production; mutations to the RNA polymerase machinery can be made to increase promoter binding affinity; deletion or overexpression of pathway or global regulators (activators and repressors); expressing mutant transcriptional regulators; and/or expressing or overexpressing ribosome recycling factor (RRF), and optionally the strain engineering comprises epigenetic modifications, optionally comprising phosphorylation, acetylation, methylation, ubiquitination, ADP-ribosylation, and/or glycosylation, and optionally the strain engineering comprises engineering in self-resistance, optionally by the upregulation or overexpression of resistance genes such as drrABC, avtAB and actAB, and optionally the strain engineering comprises genome-minimizing, optionally removing or disabling one, some, all or the majority of secondary metabolite biosynthetic gene clusters (SMBGCs), and optionally the strain engineering comprises combinatorial knockdown of secondary metabolite pathways, optionally by adding or expressing small RNAs targeting secondary metabolite biosynthesis, and optionally the strain engineering comprises expressing tRNAs for rare codons, optionally codons for AGA, AGG, AUA, CUA, GGA, CCC, and CGG, and optionally the strain engineering comprises over-expressing one or more chaperones native to strain, optionally a Streptomyces, optionally comprising over-expressing Hsp60, Hsp70, Hsp90, Hsp100, DnaK-DnaJ-GrpE and/or GroEL-GroES, e.g., to improve overall protein production, and optionally the strain engineering comprises inactivating RNaseE, optionally by mutation to enhance mRNA stability and consequently protein production, and optionally the strain engineering comprises expressing or overexpressing Streptomyces antibiotic regulatory protein (SARP) for positive regulation of antibiotic production, and optionally the strain engineering comprises expressing or overexpressing MbtH-like proteins for stimulating adenylation reactions, and optionally the strain engineering comprises expressing or overexpressing phosphopantetheinyl transferases (PPTases) proteins for stimulating post-translational modification of an apo-acyl carrier protein (apo-ACP) to activate polyketide synthases, and optionally the strain engineering comprises NAD(P)H regeneration, optionally expressing or overexpressing trans-hydrogenases for converting NADPH into NADH and NADPH+NAD+<=>NADH+NADP+.

In alternative embodiments of the products of manufacture, the cells from which the at least one cytoplasmic or nuclear extract has been derived: (a) are free of or substantially free of cell wall, cell wall components, organelles or sub-cellular compartments; or (b) are supplemented with: an organelle or sub-cellular compartment, wherein optionally the organelle comprises a natural or a synthetic Golgi organelle (optionally for glycosylation), a mitochondria or a chloroplast; a synthetic or a designer organelle, a synthetic nano- or micro-compartment, a synthetic or a designer micelle or liposome; an NAD(P)H or ATP recycling system; a mitochondria or mitochondrial extract; or a chaperone protein or a chaperone complex (optionally Hsp60, Hsp70, Hsp90, Hsp100, DnaK-DnaJ-GrpE and/or GroEL-GroES) or mbtH and its homologs, or a broad specificity 4′-Phosphopantetheine transferases or phosphoprotein phosphatase (PPTtase) including sfp and its homologs.

In alternative embodiments, the products of manufacture further comprise additional ingredients, compositions or compounds, reagents, ions or element, buffers and/or solutions, wherein optionally the additional ingredients, compositions or compounds, reagents, ions or element, buffers and/or solutions are mixed into the extract or extracts,
wherein optionally the additional ingredients, compositions or compounds, reagents, buffers and/or solutions comprise: nucleosides or nucleotides; lipids or fatty acids; carbohydrates, polysaccharides or sugars; nucleic acids or oligonucleotides; one or more enzymes, co-enzymes or enzyme co-factors; one or more amino acids; polycationic aliphatic amines or spermidine; a folinic acid, a 5-formyltetrahydrofolate or a leucovorin; a vitamin; a polyether or a polyethylene glycol (polyethylene oxide (PEO) or polyoxyethylene (POE)); a small-molecule redox reagent, an isopropyl β-D-1-thiogalactopyranoside (IPTG), a dithioerythritol (DTE) or a dithiothreitol (DTT); a glutamate or a glutamic acid; an alpha-keto amino acid or a pyruvic acid; a regulator or activator of transcription or translation, or any combination thereof,
wherein optionally the carbohydrate, polysaccharide or sugar comprises a maltodextrin, maltose, glucose and/or a hexametaphosphate (HMP); and optionally the co-enzyme or co-factor comprises an acyl-CoA precursor (optionally acetyl-CoA, malonyl-CoA, ethylmalonyl-CoA, methylmalonyl-CoA, isobutyryl-CoA, or propionyl-CoA), a nicotinamide adenine dinucleotide (NAD) or an NADH, a nicotinamide adenine dinucleotide phosphate (NADP) or an NADPH, a fluoromalonyl-CoA (F-CoA), or a S-Adenosyl methionine (SAM); and optionally the nucleosides or nucleotides comprise ATP, GTP, CTP, UTP or any combination thereof, and optionally the nucleic acids or oligonucleotides comprise transfer RNA (tRNA), small inhibitory RNA (siRNA), translational riboregulators or riboswitches; and wherein optionally the amino acid comprises non-natural amino acid (including those introduced using an “expanded genetic code”, as described by e.g., Malyshev et al., Nature 509:385, May 2014), and optionally the ion or element comprises an inorganic phosphate, a phosphonate, a phosphonic acid or a phosphonate salt; and optionally the one or more enzymes comprise an enzyme for modification of a product, a small molecule, a natural product (secondary metabolite) (NP) or a natural product analog (NPA), a protein, a lipid or fatty acid, a polysaccharide or a nucleic acid, and optionally the enzyme modification comprises: lipidation, pegylation, glycosylation, adding hydrophobic groups, myristoylation, palmitoylation, isoprenylation, prenylation, lipoylation, adding a flavin moiety (optionally comprising addition of: a flavin adenine dinucleotide (FAD) an FADH₂, a flavin mononucleotide (FMN), an FMNH₂), phospho-pantetheinylation, heme C addition, acylation, alkylation, butyrylation, carboxylation, malonylation, hydroxylation, adding a halide group, iodination, propionylation, S-glutathionylation, succinylation, glycation, adenylation, thiolation, condensation (optionally the “condensation” comprising addition of: an amino acid to an amino acid, an amino acid to a fatty acid, an amino acid to a sugar), or a combination thereof; and optionally the one or more enzymes comprise a CoA ligase, a phosphorylase, a glycosyl-transferase, a halogenase, a methyltransferase, a hydroxylase, a lambda phage GamS enzyme (optionally used with a bacterial or an E. coli extract, optionally at a concentration of about 3.5 mM), a Dsb (disulfide bond) family enzyme (optionally DsbA), or a combination thereof, or optionally the enzymes comprise one or more central metabolism enzyme (optionally tricarboxylic acid cycle (TCA, or Krebs cycle) enzymes, glycolysis enzymes or Pentose Phosphate Pathway enzymes); and optionally the additional ingredients, compositions or compounds, reagents, ions, buffers and/or solutions comprise protease, DNase or RNase inhibitors, a buffer, an anti-oxidant, a rare earths, a vitamin, a salt, a metal (optionally a trace metal, iron Fe, zinc Zn, Mg2+, Mn, vanadium) and/or a halogen; and optionally the additional ingredients, compositions or compounds, reagents, ions, buffers and/or solutions comprise a labeling agent (optionally a metabolic labeling agent), a detection or an affinity-tags, a fluorophore, reagents for biotinylation or biotin, a gold nanoparticle, an isotope or a radioactive isotope (optionally a metabolic labeling isotope, a ¹³C-lysine, a ³H thymidine, a ³⁵S methionine, a ³²P orthophosphate, a ¹⁴C-labeled D-glucose); a photoreactive amino acid label (optionally a diazirine), a bioorthogonal labeling reagent (optionally an azide, an alkyne, an aldehyde or a ketone); an RNA polymerase, optionally a T7 or T3 RNA polymerase, optionally a purified polymerase; a sigma factor, optionally a Streptomyces sigma factor or, a sigma 70, a sigma 54, sigma factors HrdB, 19, 24, 28, 32 and/or 38); and optionally the additional ingredients, compositions or compounds, reagents, ions, buffers and/or solutions comprise one, several or all of the additional ingredients, compositions, compounds or reagents as set forth in Table 1, and optionally at the concentration set forth in Table 1.

In alternative embodiments of the products of manufacture, the nucleic acids comprise: a substantially isolated or a synthetic nucleic acid comprising or encoding: an enzyme-encoding natural-product (NP)- or natural product analog (NPA)-synthesizing operon; a biosynthetic gene cluster, optionally a biosynthetic gene cluster comprising coding sequence for all or substantially all enzymes needed in the synthesis of a natural product (NP), NPA, or a secondary metabolite; a plurality of enzyme-encoding nucleic acids; a plurality of enzyme-encoding nucleic acids for at least two, several or all of the steps in the synthesis of a product or chemical or molecule, wherein optionally the product or chemical is a natural product (NP) or a natural product analog (NPA); wherein optionally the chemical, the natural product (NP), the NPA, or secondary metabolite is: a violacein, a butadiene, a propylene, a 1,4-butanediol, a 1,3-butanediol, a crotyl alcohol, a methyl vinyl carbinol, an isopropanol, an ethylene glycol, a terephthalic acid, an adipic acid, a hexamethylenediamine (HMDA), a caprolactam, a cyclohexanone, a aniline, a Methyl Ethyl Ketone (MEK), a fatty alcohol, an acrylic acid, an acrylate ester, a methyl methacrylate, a lipid, a carbohydrate, a beta-lactam, a polyketide, a macrolide, a macrolide having a 14-, 15- or 16-membered macrocyclic lactone ring, a trans-AT type I PKS, a Type II PKS, or a Type III PKS, a heterocyst glycolipid PKS-like, a cyclic peptide, a bottromycin or an antibiotic, and optionally the chemical, the natural product (NP), the NPA, or secondary metabolite is a terpenoid, a steroid, an alkaloid, a fatty acid, a nonribosomal polypeptide (“NRP”; also referred to as “nonribosomal peptide”), an enzyme cofactor, an aminocoumarin, a melanin, an aminoglycosides/aminocyclitol, a microcin, an aryl polyene, a microviridin, a bacteriocin, a nucleoside, an oligosaccharide, a butyrolactone, a phenazine, a phosphoglycolipid, a cyanobactin, a phosphonate, a (dialkyl)resorcinol, a polyunsaturated fatty acid, an ectoine, a furan, a lycocin, a Head-to-tail cyclized peptide, a proteusin, a homoserine lactone, a sactipeptide, an indole, a siderophore, a ladderane lipid, a terpene, a lantipeptide, a thiopeptide, a linear azol(in)e-containing peptides (LAPs), a lasso peptide, a linaridin; and optionally the substantially isolated or a synthetic nucleic acid are in a linear or a circular form; and optionally the substantially isolated or a synthetic nucleic acid is contained in a circular or a linearized plasmid, vector or phage DNA; and optionally the substantially isolated or a synthetic nucleic acid comprises enzyme coding sequences operably linked to a homologous or a heterologous transcriptional regulatory sequence, optionally a transcriptional regulatory sequence is a promoter, an enhancer, or a terminator of transcription; and optionally the substantially isolated or a synthetic nucleic acid comprises has at least about 100, 200, 300, 400 or 500 or more base pair ends upstream of the promoter and/or downstream of the terminator; and optionally the promoter comprises a native promoter (a promoter used in an organism that is a source of the mixed extract) or a synthetic promoter, and optionally promoters operably linked to nucleic acids comprise a combination of promoters from all or several of the organisms that are the source of the mixed extract (optionally an E. coli and a Streptomyces extract, and a combination of E. coli and a Streptomyces promoter are used) (taking advantage of the available transcriptional machinery from E. coli and Streptomyces as well as a bacteriophage orthogonal RNA polymerase); and optionally the promoter is ermEp* (a heterologous promoter from Saccharopolyspora erythraea and is active in E. coli), SF14p, or kasOp* (active in E. coli); and optionally all, or a subset, of the enzyme-encoding nucleic acid of the enzyme-encoding natural-product (NP), NPA, or a synthesizing operon or biosynthetic gene cluster are contained on separate linear nucleic acids (separate nucleic acid strands), optionally in equimolar concentrations in the mixed cytoplasmic or nuclear extract, and optionally, each separate linear nucleic acid comprises one, two, three, 4, 5, 6, 7, 8, 9, or 10 or more genes or enzyme-encoding sequences, and optionally the linear nucleic acid is present in a concentration of about 1.0 nM (nanomolar), 5 nM, nM, 15 nM, 20 nM, 25 nM, 30 nM, 35 nM, 40 nM, 45 nM or 50 nM or more or between about 1 nM and 100 nM; and optionally the enzyme-encoding nucleic acids, the linear nucleic acid or all, or a subset, of the enzyme-encoding nucleic acid of the enzyme-encoding natural-product synthesizing operon or biosynthetic gene cluster are immobilized, optionally immobilized on a bead or a chip; and optionally the enzyme encoded by the nucleic acid are between about 10 and 100 kDa, or about 10 kDa, 20 kDa, 30 kDa, 40 kDa, 50 kDa, 60 kDa, 70 kDa, 80 kDa, 90 kDa, or 100 kDa, 110 kDa, 120 kDa, 130 kDa, 140 kDa, 150 kDa, or more;
and optionally the substantially isolated or a synthetic nucleic acid comprises: (i) a genome, a gene or a DNA from a source other than the cell used for the extract (an exogenous nucleic acid), or an exogenous nucleic acid that has been engineered or mutated, optionally engineered or mutated in a protein coding region or in a non-coding region, (ii) a genome, a gene or a DNA from a cell used for the extract (an endogenous nucleic acid), or an endogenous nucleic acid that has been engineered or mutated, optionally engineered or mutated in a protein coding region or in a non-coding region, (iii) a genome, a gene or a DNA from one, both or several of the organisms used as a source for the extract, or, (iv) any or all of (i) to (iii).

In alternative embodiments, of the product of manufacture, the enzyme-encoding natural-product (NP), natural product analog (NPA), or synthesizing operon; the biosynthetic gene cluster; or the plurality of enzyme-encoding nucleic acids for at least two, several or all of the steps in the synthesis of a natural product (NP), natural product analog (NPA) or secondary metabolite or a chemical or molecule: (a) comprise one or several cryptic, or phenotypically silent, genes, optionally identified by a software or sequence analysis of a genome, wherein optionally the program is antiSMASH (ANTISMASH™); (b) are genetically modified, optionally modified for optimization of transcription, translation and/or function of an encoded protein; and optionally translation efficiency of mRNA sequences is determined by RBSDesigner (RBSDESIGNER™) and RNA encoding sequence are optimized to sequences determined by RBSDesigner; and optionally the enzyme-encoding natural-product (NP) or natural product analog (NPA)-synthesizing operon; the biosynthetic gene cluster; or the plurality of enzyme-encoding nucleic acids for at least two, several or all of the steps in the synthesis of a natural product (NP), natural product analog (NPA) or secondary metabolite or a chemical or molecule are identified by methods comprising use of: a genomic or biosynthetic search engine, optionally WARP DRIVE BIO™ software, antiSMASH (ANTISMASH™) software, iSNAP™, CgustScan™, NP.searcher™ SBSPKS™, BAGEL3™, SMURF™, ClusterFinder (CLUSTERFINDER™) or ClusterBlast (CLUSTERBLAST™) algorithms, or a combination thereof, or, an Integrated Microbial Genomes (IMG)-ABC system (DOE Joint Genome Institute (JGI)).

In alternative embodiments, provided are processes for in vitro, or cell free, transcription/translation, comprising: (a) providing a product of manufacture as provided herein; (b) providing a substantially isolated or a synthetic nucleic acid, wherein the nucleic acid is an RNA or a DNA or a synthetic analog thereof, or the nucleic acid is linear or circular, wherein optionally the substantially isolated or a synthetic nucleic acid or synthetic analog thereof comprises or is derived from: (i) a genome, a gene or a DNA from a source other than the cell used for the extract (an exogenous nucleic acid), or an exogenous nucleic acid that has been engineered or mutated, optionally engineered or mutated in a protein coding region or in a non-coding region, (ii) a genome, a gene or a DNA from a cell used for the extract (an endogenous nucleic acid), or an endogenous nucleic acid that has been engineered or mutated, optionally engineered or mutated in a protein coding region or in a non-coding region, (iii) a genome, a gene or a DNA from one, both or several of the organisms used as a source for the extract, or (iv) any or all of (i) to (iii); and, (c) mixing the product of manufacture and the substantially isolated or a synthetic nucleic acid under conditions wherein an RNA is transcribed from the nucleic acid and a corresponding protein is translated from the RNA, or when the provided nucleic acid is RNA then a corresponding protein is translated from the RNA.

In alternative embodiments of the processes, the substantially isolated or a synthetic nucleic acid comprises: an enzyme-encoding operon; a biosynthetic gene cluster; a plurality of enzyme-encoding nucleic acids; a plurality of enzyme-encoding nucleic acids for at least two, several or all of the steps in the synthesis of a product or a chemical, wherein optionally the product or chemical is a natural product (NP), natural product analog (NPA) or secondary metabolite.

In alternative embodiments of the processes, the natural product (NP), natural product analog (NPA) or secondary metabolite is or comprises: a violacein, a butadiene, a propylene, a 1,4-butanediol, an isopropanol, an ethylene glycol, a terephthalic acid, an adipic acid, a hexamethylenediamine (HMDA), a caprolactam, a cyclohexanone, a aniline, a Methyl Ethyl Ketone (MEK), a fatty alcohol, an acrylic acid, an acrylate ester, a methyl methacrylate, a lipid, a carbohydrate, or an antibiotic; a butadiene, a propylene, a 1,4-butanediol, a 1,3-butanediol, a crotyl alcohol, a methyl vinyl carbinol, an isopropanol, an ethylene glycol, a terephthalic acid, an adipic acid, a hexamethylenediamine (HMDA), a caprolactam, a caprolactone, a hexanediol, a cyclohexanone, an aniline, a Methyl Ethyl Ketone (MEK), a fatty alcohol, an acrylic acid, an acrylate ester, a methyl methacrylate, a lipid, a carbohydrate, a beta-lactam, a polyketide, a macrolide, a macrolide having a 14-, 15- or 16-membered macrocyclic lactone ring, a ketolide, a taxane, a trans-AT type I PKS, a Type II PKS, or a Type III PKS, a heterocyst glycolipid PKS-like, a cyclic peptide, or a bottromycin, a terpenoid, a steroid, an alkaloid, a fatty acid, a nonribosomal polypeptide, an enzyme cofactor, an aminocoumarin, a melanin, an aminoglycosides/aminocyclitol, a microcin, an aryl polyene, a microviridin, a bacteriocin, a nucleoside, an oligosaccharide, a butyrolactone, a phenazine, a phosphoglycolipid, a cyanobactin, a phosphonate, a (dialkyl)resorcinol, a polyunsaturated fatty acid, an ectoine, a furan, a lycocin, a Head-to-tail cyclized peptide, a proteusin, a homoserine lactone, a sactipeptide, an indole, a siderophore, a ladderane lipid, a terpene, a lantipeptide, a thiopeptide, a linear azol(in)e-containing peptides (LAPs), a lasso peptide, a linaridin; a natural product (NP) or national product analog (NPA) useful for human and animal health and nutrition or crop health,
optionally comprising antibacterial agents, antifungal agents, cytotoxins, anticancer and antitumor agents, immunomodulators, anti-inflammatory, anti-arthritic, anthelminthic, insecticides, coccidiostats and anti-diarrhea agents; optionally comprising: a cytotoxin, an aminoglycoside antibiotic, a macrolide polyketide (Type I PKS), an oligopyrrole, a nonribosomal peptide, an aromatic polyketide (optionally an aromatic polyketide of a Type III PKS, an aromatic polyketide of Type II PKS), a complex isoprenoid, a beta-lactam, a terpenoid, a hybrid peptide-polyketide (from Type I PKS and NRPS), and/or a taxane; optionally comprising an antibacterial compound, optionally comprising an antibacterial compound, optionally a vancomycin, erythromycin, daptomycin; antifungal agents (optionally amphotericin, nystatin); anticancer and antitumor agents for example doxorubicin, bleomycin; immunomodulators or immunosuppressants for example rapamycin, tacrolimus; anti-inflammatory or anti-arthritic compounds for example ginsenosides including ginsenoside compound K, Rh2, Rh1, Rg5, Rk1, Rg2, Rg3, Rg1, Rf, Re, Road, Rb2, Rc and Rb1; anthelminthics for example avermectins; insecticides for example spinosyns; coccidiostats for example monensin, narasin; animal health compounds for example avilamycin, tilmicosin;
optionally comprising acetogenins, actinorhodine, aflatoxin, albaflavenone, amphotericin, amphotericin b, annonacin, ansamycins, anthramycin, antihelminthics, avermectin, avilamycin, azithromycin, bleomycin, bullatacin, caprazamycins, carbomycin a, cephamycin c, cethromycin, chartreusin, calicheamicin, chloramphenicol, clarithromycin, clavulanate, coelchelin, cytotoxins, daptomycin, discodermolide, doxycycline, daunomycin, docetaxel, dolastatin, doxorubicin, echinomycin, endophenazine, epithienamycin, erythromycin, erythromycin a, fidaxomicin, FK506, flaviolin, fredericamycin, geldanamycin, ginsenoside compound K, Rh2, Rh1, Rg5, Rk1, Rg2, Rg3, Rg1, Rf, Re, Road, Rb2, Rc and Rb, geosmin, glucosyl-a47934, iso-migrastatin, ivermectin, josamycin, ketolides, kitasamycin, lovastatin, macbecin, macrolides, macrotetrolide, midecamycin, molvizarin, monensin, napyradiomycin, narasin, novobiocin, nystatin, oleandomycin, oxytetracycline, paclitaxel, pentalenolactone, phenalinolactione, pikromycin, pimaricin, pimecrolimus, polyene antimycotics, polyenes, polyketide macrolides, polyketides, radicicol, rapamycin, rifamycin, roxithromycin, sirolimus, solithromycin, spinosad, spinosyns, spiramycin, squamocin, staurosporine, streptomycin, tacrolimus, telithromycin, tetracenomycin, tetracyclines, teixobactin, thiocoraline, tilmicosin, troleandomycin, tylocine, tylosin, undecylprodigiosin, usnic acid, uvaricin, vancomycin and analogs thereof.

In alternative embodiments the processes further comprise making a natural product (NP) analog library or secondary metabolite analog library by subjecting the substantially isolated or a synthetic nucleic acid (optionally comprising an enzyme-encoding operon; a biosynthetic gene cluster; a plurality of enzyme-encoding nucleic acids; a plurality of enzyme-encoding nucleic acids for at least two, several or all of the steps in the synthesis of a product or a chemical, optionally a natural product) to one or more combinatorial modifications to generate the natural product analog library, or to generate diversity in the natural product (NP), NP analog (NPA) or secondary metabolite analog library, wherein optionally the one or more combinatorial modifications comprise: (a) deletion or inactivation of a module in a gene cluster for the biosynthesis of the natural product (NP) or secondary metabolite, or the NP analog (NPA) or secondary metabolite analog, (b) domain engineering to fuse domains, shuffling of domains, addition of an extra domain, exchange of multiple domains, or other modifications to alter substrate activity or specificity of an enzyme involved in the biosynthesis or modification of the natural product (NP) or secondary metabolite, or the NP analog (NPA) or secondary metabolite analog, (c) modifying a tailoring enzyme that acts after the biosynthesis of the core backbone or the natural product (NP) or secondary metabolite is completed, optionally comprising a methyl transferase, a glycosyl transferase, a halogenase, a hydroxylase, a dehydrogenase, (d) gene exchanges, gene deletions (exclusions), module exchanges, multi-domain exchanges, lipid side chain changes, addition of tailoring enzymes and the overexpression of enzymes for post-translational modifications, (e) combining modules from various sources to construct an artificial gene synthesis clusters, or, (f) and combination or all of (a) to (e);
wherein optionally the one or more combinatorial modifications to generate the natural product analog or secondary metabolite analog library, or to generate diversity in the natural product library, comprises refactoring a natural product gene cluster and/or a biosynthetic gene, optionally the refactoring comprising replacing the native regulatory parts (e.g. a promoter, RBS, terminator, codon usage etc. of the native or originating enzyme-encoding operon, biosynthetic gene cluster, plurality of enzyme-encoding nucleic acids, plurality of enzyme-encoding nucleic acids for at least two, several or all of the steps in the synthesis of a product or a chemical, optionally a natural product) with synthetic, orthogonal regulation, optionally with the goal of optimization of enzyme expression in a mixed extract product of manufacture and/or in a heterologous host,
and optionally the modifications comprise protein engineering, which optionally comprises: (i) generation of structural diversity of NP analogs or secondary metabolite analogs, optionally polyketide synthases (PKSs) and analogs thereof, by incorporating different starter and extender acyl units, (ii) mutagenesis to increase the diversity of NP analogs or secondary metabolite analogs applied to active site residues, optionally PKS active site residues, optionally incorporating different starter and extender units, (iii) mutagenesis for changing or controlling chain length of NP analogs or secondary metabolite analogs, optionally polyketides, (iv) mutagenesis or one or more or all domain(s) of an NP analog or a secondary metabolite analog, optionally PKS domains, optionally comprising the AT: Acyltransferase, ACP: Acyl carrier protein with an SH group on the cofactor, a serine-attached 4′-phosphopantetheine, KS: Keto-synthase with an SH group on a cysteine side-chain, KR: Ketoreductase, DH: Dehydratase, ER: Enoylreductase, MT: Methyltransferase O— or C— (α or β), SH: Sulfhydrolase, TE: Thioesterase, domain, (v) engineering of one or more or all NP analog or a secondary metabolite analog domains, optionally PKS domains, or the reductive domains, of PKSs, optionally the KR and ER domains, optionally to have different region-and-stereospecificities, optionally engineering one or more or all domain, optionally PKS domains, through mutagenesis to produce analogs with precise modifications and stereochemistry (using protocols as described in e.g., Zabala et al., Ind Microbiol Biotechnol. 2012; 39:227-241), (vi) mutagenesis strategies to inactivate NP analog or a secondary metabolite analog domains, optionally PKS KR domains, which can alter the regio- and stereospecificities of the NP analog or a secondary metabolite analog, e.g., PKS, (v) mutagenesis of NRPS (non-ribosomal peptide synthetases) modules or domains, optionally the adenylation domain (A), the thiolation domain (T), and/or the condensation domain (C); or inactivation of individual domains in the large NRPS (can result in the predictive synthesis of unnatural analogues), (vi) mutagenesis of NRPS adenylation domains, optionally altering the specificity of the loading module to accept different amino acids, (vii) applying directed evolution techniques on chimeric NRPSs with swapped domains and modules, optionally protocols as described in Fischbach et al., Proc Natl Acad Sci USA. 2008; 105:4601-4608, (viii) use of ssrA tags or Transfer-messenger RNA (tmRNA) (optionally ClpXP, ClpAP degradation tags), to selectively add to domains of interest, or addition of an SsrA-SmpB system for protein tagging, directed degradation and/or ribosome rescue, or, (ix) any or all of (i) to (viii).

In alternative embodiments provided are methods for screening for: a modulator of protein activity, transcription or translation or cell function; a toxic metabolite or a protein; a cellular toxin; an inhibitor of transcription or translation, comprising: (a) providing a product of manufacture as provided herein, wherein the product of manufacture comprises at least one protein-encoding nucleic acid; (b) providing a test compound; (c) combining or mixing the test compound with the product of manufacture under conditions wherein the extract initiates or completes transcription and/or translation, or modifies a molecule (optionally a protein, a small molecule, a natural product (NP), natural product analog (NPA) or secondary metabolite or a lipid) and (d) determining or measuring any change in the functioning or products of the extract, or the transcription and/or translation, wherein determining or measuring a change in the protein activity, transcription or translation or cell function identifies the test compound as a modulator of that protein activity, transcription or translation or cell function.

In alternative embodiments provided are in vitro methods for making, synthesizing or altering the structure of a compound, composition, organic molecule small molecule or natural product (NP), natural product analog (NPA) or secondary metabolite or library thereof, comprising using a mixture as provided herein or by using a process as provided herein; and optionally at least two or more of the altered compounds are synthesized to create a library of altered compounds; and optionally the library is a natural product analog library.

In alternative embodiments provided are libraries of: natural products (NPs) or natural product analogs (NPAs), or structural analogs of a secondary metabolite, or a combination thereof, prepared, synthesized or modified by a method comprising use of a product of manufacture or the extract mixture as provided herein, or by using a process or method as provided herein. In alternative embodiments, exemplary libraries are made by methods comprising preparing, synthesizing or modifying the natural products or natural product analogs, or structural analogs of the secondary metabolite, or the combination thereof, comprises using an extract from an Escherichia and from an Actinomyces, optionally a Streptomyces.

In alternative embodiments of the libraries, at least one natural product or natural product analog, or structural analog of the secondary metabolite, is fused or conjugated to a carrier molecule, optionally a pharmaceutically acceptable carrier molecule, optionally a polymer, a protein or peptide, an antibody or fragment thereof, an affibody, a PEG or a PEG derivative, a lipophilic carrier including a fatty acid, optionally palmitoyl, myristoyl, stearic acid, 3-pentadecylglutaric acid, that associates with a serum protein such as albumin, LDL or HDL, and wherein optionally the carrier increases blood circulation time or cell-targeting or both for the NP or NPA. In alternative embodiments of the libraries, the natural product (NP) or NPA, or structural analog of the secondary metabolite: is fused or conjugated to the carrier in the extract, and optionally is enriched before being fused or conjugated to the carrier, or is isolated before being fused or conjugated to the carrier. In alternative embodiments of the libraries, the NP or NP, or structural analog of the secondary metabolite, is site-specifically fused or conjugated to the carrier, optionally wherein the NP or NPA, or structural analog of the secondary metabolite, is modified to comprise a group capable of the site-specific fusion or conjugation to the carrier, optionally where the NP or NPA, or structural analog of the secondary metabolite, is synthesized in the extract to comprise the site-specific reactive group, and optionally wherein the library contains a plurality of NP or NPA, or structural analogs of the secondary metabolite, each having a site-specific reactive group at a different location on the NP or NPA, or structural analog of the secondary metabolite.

In alternative embodiments of the libraries, the site-specific reactive group can react with a cysteine or lysine or glutamic acid on the carrier. In alternative embodiments of the libraries, the natural product analogs (NPAs) or structural analogs of the secondary metabolite, or the diversity of natural product analogs (NPAs) or structural analogs of the secondary metabolite, is generated by a process comprising modifying the natural product (NP) or secondary metabolite chemically or by enzyme modification, wherein optionally the enzyme modification comprises modification of the natural product (NP) or structural analog of the secondary metabolite by: halogenation, lipidation, pegylation, glycosylation, adding hydrophobic groups, myristoylation, palmitoylation, isoprenylation, prenylation, lipoylation, adding a flavin moiety (optionally comprising addition of: a flavin adenine dinucleotide (FAD) an FADH₂, a flavin mononucleotide (FMN), an FMNH₂), phospho-pantetheinylation, heme C addition, acylation, alkylation, butyrylation, carboxylation, malonylation, hydroxylation, adding a halide group, iodination, propionylation, S-glutathionylation, succinylation, glycation, adenylation, thiolation, condensation (optionally the “condensation” comprising addition of: an amino acid to an amino acid, an amino acid to a fatty acid, an amino acid to a sugar), or a combination thereof, and optionally the enzyme modification comprises modification of the natural product (NP) or structural analog of the secondary metabolite by one or more enzymes comprising: a CoA ligase, a phosphorylase, a glycosyl-transferase, a halogenase, a methyltransferase, a hydroxylase, a lambda phage GamS enzyme (optionally used with a bacterial or an E. coli extract, optionally at a concentration of about 3.5 mM), a Dsb (disulfide bond) family enzyme (optionally DsbA), or a combination thereof, or optionally the enzymes comprise one or more central metabolism enzyme (optionally tricarboxylic acid cycle (TCA, or Krebs cycle) enzymes, glycolysis enzymes or Pentose Phosphate Pathway enzymes), and optionally the chemical or enzyme modification comprises addition, deletion or replacement of a substituent or functional groups, optionally a hydroxyl group, an amino group, a halogen, an alkyl or a cycloalkyl group, optionally by hydration, hydrogenation, an Aldol condensation reaction, condensation polymerization, halogenation, oxidation, dehydrogenation, or creating one or more double bonds, and optionally wherein the chemical or enzyme modification comprises altering a gene, a gene cluster or operon encoding the enzyme or enzyme.

In alternative embodiments, provided are compositions comprising: a natural product (NP) or natural product analog (NPA) or structural analog of the secondary metabolite, obtained from a library as provided herein, wherein optionally the composition further comprises, is formulated with, or is contained in: a liquid, a solvent, a solid, a powder, a bulking agent, a filler, a polymeric carrier or stabilizing agent, a liposome, a particle or a nanoparticle, a buffer, a carrier, a delivery vehicle, or an excipient, optionally a pharmaceutically acceptable excipient. The natural product (NP) or natural product analog (NPA) can be fused or conjugated to a carrier molecule, optionally a pharmaceutically acceptable carrier molecule, optionally a polymer, a protein or peptide, an antibody or fragment thereof, an affibody, a PEG or a PEG derivative, a lipophilic carrier including a fatty acid, optionally palmitoyl, myristoyl, stearic acid, 3-pentadecylglutaric acid, that associates with a serum protein such as albumin, LDL or HDL, and wherein optionally the carrier increases blood circulation time or cell-targeting or both for the NP or NPA. The natural product or NPA can be fused or conjugated to the carrier in the extract, and optionally is enriched before being fused or conjugated to the carrier, or is isolated before being fused or conjugated to the carrier. The NP or NP can be site-specifically fused or conjugated to the carrier, optionally wherein the NP or NPA is modified to comprise a group capable of the site-specific fusion or conjugation to the carrier, optionally where the NP or NPA is synthesized in the extract to comprise the site-specific reactive group, and optionally wherein the library contains a plurality of NP or NPA each having a site-specific reactive group at a different location on the NP or NPA. The site-specific reactive group can react with a cysteine or lysine or glutamic acid on the carrier.

In alternative embodiments, provided are products of manufacture comprising: (a) at least one cytoplasmic extract or at least one nuclear extract, wherein optionally the at least one cytoplasmic extract or nuclear extract comprises a second extract (to result in an extract mixture), and optionally the extract mixture comprises at least two cytoplasmic extracts; at least two nuclear extracts; or at least one cytoplasmic and one nuclear extract, from at least two different cells, wherein optionally the at least one extract or extract mixture is capable of in vitro coupled transcription and translation; and (b) a substantially isolated or a synthetic nucleic acid comprising or encoding: an enzyme-encoding natural-product (NP)- or natural product analog (NPA)-synthesizing operon; a biosynthetic gene cluster, optionally a biosynthetic gene cluster comprising coding sequence for all or substantially all enzymes needed in the synthesis of a natural product (NP), NPA, or a secondary metabolite; a plurality of enzyme-encoding nucleic acids; a plurality of enzyme-encoding nucleic acids for at least two, several or all of the steps in the synthesis of a product or chemical or molecule, wherein optionally the product or chemical is a natural product (NP) or a natural product analog (NPA) or secondary metabolite analog;
wherein optionally the enzyme-encoding natural-product (NP), natural product analog (NPA), or synthesizing operon; the biosynthetic gene cluster; or the plurality of enzyme-encoding nucleic acids for at least two, several or all of the steps in the synthesis of a natural product (NP), natural product analog (NPA) or secondary metabolite analog, or chemical or molecule: (i) comprise one or several cryptic, or phenotypically silent, genes, optionally identified by a software or sequence analysis of a genome, wherein optionally the program is antiSMASH (ANTISMASH™), or (ii) are genetically modified, optionally modified for optimization of transcription, translation and/or function of an encoded protein, and optionally translation efficiency of mRNA sequences is determined by RBSDesigner (RBSDESIGNER™), and RNA encoding sequence are optimized to sequences determined by RBSDesigner, and optionally the enzyme-encoding natural-product (NP) or natural product analog (NPA)-synthesizing operon; the biosynthetic gene cluster; or the plurality of enzyme-encoding nucleic acids for at least two, several or all of the steps in the synthesis of a natural product (NP), natural product analog (NPA) or secondary metabolite or a chemical or molecule are identified by methods comprising use of: a genomic or biosynthetic search engine, optionally WARP DRIVE BIO™ software, antiSMASH (ANTISMASH™) software, iSNAP™, CgustScan™, NP.searcher™, SBSPKS™, BAGEL3™ SMURF™, ClusterFinder (CLUSTERFINDER™) or ClusterBlast (CLUSTERBLAST™) algorithms, or a combination thereof, or, an Integrated Microbial Genomes (IMG)-ABC system (DOE Joint Genome Institute (JGI)).

In alternative embodiments the products of manufacture further comprise subjecting the substantially isolated or a synthetic nucleic acid (optionally comprising an enzyme-encoding operon; a biosynthetic gene cluster; a plurality of enzyme-encoding nucleic acids; a plurality of enzyme-encoding nucleic acids for at least two, several or all of the steps in the synthesis of a product or a chemical, optionally a natural product, natural product analog (NPA) or secondary metabolite analog) to one or more combinatorial modifications (optionally to generate the natural product analog library, or to generate diversity in the natural product library),
wherein optionally the one or more combinatorial modifications comprise: (a) deletion or inactivation of a module in a gene cluster for the biosynthesis of the natural product (NP) or secondary metabolite, or the NP analog (NPA) or secondary metabolite analog, (b) domain engineering to fuse domains, shuffling of domains, addition of an extra domain, exchange of multiple domains, or other modifications to alter substrate activity or specificity of an enzyme involved in the biosynthesis or modification of the natural product (NP) or secondary metabolite, or the NP analog (NPA) or secondary metabolite analog, (c) modifying a tailoring enzyme that acts after the biosynthesis of the core backbone or the natural product (NP) or secondary metabolite is completed, optionally comprising a methyl transferase, a glycosyl transferase, a halogenase, a hydroxylase, a dehydrogenase, (d) gene exchanges, gene deletions (exclusions), module exchanges, multi-domain exchanges, lipid side chain changes, addition of tailoring enzymes and the overexpression of enzymes for post-translational modifications, (e) combining modules from various sources to construct an artificial gene synthesis clusters, or, (f) and combination or all of (a) to (e);
wherein optionally the one or more combinatorial modifications to generate the natural product analog or secondary metabolite analog library, or to generate diversity in the natural product library, comprises refactoring a natural product gene cluster and/or a biosynthetic gene, optionally the refactoring comprising replacing the native regulatory parts (optionally a promoter, RBS, terminator, codon usage, and equivalents, of the native or originating enzyme-encoding operon, biosynthetic gene cluster, plurality of enzyme-encoding nucleic acids, plurality of enzyme-encoding nucleic acids for at least two, several or all of the steps in the synthesis of a product or a chemical, optionally a natural product) with synthetic, orthogonal regulation, optionally with the goal of optimization of enzyme expression in a mixed extract product of manufacture and/or in a heterologous host;
and optionally the diversity of the structural analogs of the NP, NPA or secondary metabolite is increased by protein engineering approaches, or the compound (NP or NPA) or protein modifications comprise protein engineering approaches, which optionally comprise: (i) generation of structural diversity of polyketide synthases (PKSs) by incorporating different starter and extender acyl units, (ii) mutagenesis to increase the diversity, optionally applied to PKS active site residues towards incorporating different starter and extender units, or for changing or controlling chain length of polyketides, (iii) mutagenesis or one or more or all PKS domains, optionally comprising the AT: Acyltransferase, ACP: Acyl carrier protein with an SH group on the cofactor, a serine-attached 4′-phosphopantetheine, KS: Keto-synthase with an SH group on a cysteine side-chain, KR: Ketoreductase, DH: Dehydratase, ER: Enoylreductase, MT: Methyltransferase O— or C— (α or β), SH: Sulfhydrolase, TE: Thioesterase, domain, (iv) engineering of one or more or all PKS domains, or the reductive domains, of PKSs, optionally the KR and ER domains, e.g., to have different region-and-stereospecificities, optionally engineering one or more or all PKS domains through mutagenesis to produce analogs with precise modifications and stereochemistry (using protocols as described in e.g., Zabala et al., Ind Microbiol Biotechnol. 2012; 39:227-241), (v) mutagenesis strategies to inactivate KR domains which can alter the regio- and stereospecificities of the PKSs, (vi) mutagenesis of NRPS (non-ribosomal peptide synthetases) modules or domains, optionally the adenylation domain (A), the thiolation domain (T), and/or the condensation domain (C); or inactivation of individual domains in the large NRPS (can result in the predictive synthesis of unnatural analogues); (vii) mutagenesis of NRPS adenylation domains (can alter the specificity of the loading module to accept different amino acids), (viii) applying directed evolution techniques on chimeric NRPSs with swapped domains and modules (optionally protocols as described in Fischbach et al., Proc Natl Acad Sci USA. 2008; 105:4601-4608), (ix) use of ssrA tags or Transfer-messenger RNA (tmRNA) (optionally ClpXP, ClpAP degradation tags), can be selectively added to domains of interest, or addition of an SsrA-SmpB system for protein tagging, directed degradation and/or ribosome rescue; or, (x) any combination or all of (i) to (ix).

In alternative embodiments of the products of manufacture the natural product analogs (NPAs) or structural analogs of the secondary metabolite, or the diversity of natural product analogs (NPAs) or structural analogs of the secondary metabolite, are generated by, or are further modified by, a process comprising modifying the natural product (NP) or secondary metabolite (or further modifying an NP analog or secondary metabolite analog) chemically or by enzyme modification, wherein optionally the enzyme modification comprises modification of the natural product (NP) or structural analog of the secondary metabolite by: halogenation, lipidation, pegylation, glycosylation, adding hydrophobic groups, myristoylation, palmitoylation, isoprenylation, prenylation, lipoylation, adding a flavin moiety (optionally comprising addition of: a flavin adenine dinucleotide (FAD) an FADH₂, a flavin mononucleotide (FMN), an FMNH₂), phospho-pantetheinylation, heme C addition, acylation, alkylation, butyrylation, carboxylation, malonylation, hydroxylation, adding a halide group, iodination, propionylation, S-glutathionylation, succinylation, glycation, adenylation, thiolation, condensation (optionally the “condensation” comprising addition of: an amino acid to an amino acid, an amino acid to a fatty acid, an amino acid to a sugar), or a combination thereof, and optionally the enzyme modification comprises modification of the natural product (NP) or structural analog of the secondary metabolite by one or more enzymes comprising: a CoA ligase, a phosphorylase, a glycosyl-transferase, a halogenase, a methyltransferase, a hydroxylase, a lambda phage GamS enzyme (optionally used with a bacterial or an E. coli extract, optionally at a concentration of about 3.5 mM), a Dsb (disulfide bond) family enzyme (optionally DsbA), or a combination thereof, or optionally the enzymes comprise one or more central metabolism enzyme (optionally tricarboxylic acid cycle (TCA, or Krebs cycle) enzymes, glycolysis enzymes or Pentose Phosphate Pathway enzymes), and optionally the chemical or enzyme modification comprises addition, deletion or replacement of a substituent or functional groups, optionally a hydroxyl group, an amino group, a halogen, an alkyl or a cycloalkyl group, optionally by hydration, hydrogenation, an Aldol condensation reaction, condensation polymerization, halogenation, oxidation, dehydrogenation, or creating one or more double bonds, and optionally wherein the chemical or enzyme modification comprises altering a gene, a gene cluster or operon encoding the enzyme or enzyme.

In alternative embodiments of the products of manufacture the at least two different cells are from different kingdoms, phyla, classes, orders, families, genera or species; or the at least two different cell extracts comprise at least one extract from: a prokaryotic or a eukaryotic cell; or, a bacterial cell, a fungal cell, a yeast cell, an algae cell, an Archaeal cell, an insect cell, a plant cell, a mammalian cell or a human cell. The at least one cytoplasmic extract or at least one nuclear extract, or the extract mixture thereof, can comprise: (a) an undiluted liquid isolate, optionally from at least one of the at least two different cells; (b) a diluted liquid preparation, optionally from at least one of the at least two different cells, wherein optionally the cytoplasmic or nuclear extract or combined cytoplasmic and nuclear extract is diluted with a saline or a buffer; (c) an undiluted liquid preparation, optionally from at least one of the at least two different cells; or (d) a lyophilized preparation of the mixture of at least two cytoplasmic or nuclear extracts or combined cytoplasmic and nuclear extract from at least two different cells.

In alternative embodiments of the products of manufacture: (a) between about 50% and 99.9% or about 50%, 51%, 52%5, 53% 54%, 55%, 56%, 57%, 58%5, 59% 60%, 61%, 62%, 63%, 64%, 65%, 66%, 67%, 68%, 69%0, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 99.5% of the liquid volume of the extract is from one of the two extracts (from one of the cytoplasmic or nuclear or combined cytoplasmic and nuclear extracts); (b) the mixture of at least two cytoplasmic or nuclear or combined cytoplasmic and nuclear extracts comprises: at least one extract from a prokaryotic cell or a bacterial cell and at least one extract from a eukaryotic cell; at least one extract from a prokaryotic cell and at least one extract from a mammalian cell; at least one extract from a bacterial cell and at least one extract from an insect, a plant, a fungal or a yeast cell; or, extracts from at least two different bacterial cells, two different fungal cells; two different yeast cells, two different insect cells, two different plant cells or two different mammalian cells; (c) the mixture of at least two cytoplasmic or nuclear or combined cytoplasmic and nuclear extracts comprises a mixture of a cytoplasmic and a nuclear extract; a mixture of two different cytoplasmic extracts; or a mixture of at least two different nuclear extracts; (d) at least one of the cytoplasmic or nuclear extracts, or combined cytoplasmic and nuclear extracts, comprises an extract from or an extract derived from: a prokaryote (optionally a bacteria, an Archaea), a eukaryote (optionally a fungi, a plant, an animal, a human), a bacterial isolate from an environmental source or sample, a Saccharomyces cerevisiae or a yeast, a Aspergillus or fungus, optionally A. oryzae, A. nidulans, a plant or plant product, optionally a wheat germ, P. somniferum S. lycopersicon, M esculenta, L. japonicas, A. thaliana, Zea mays, Avena spp, an Escherichia or a Escherichia coli (E. coli); an Actinomyces or a Streptomyces or an Actinobacteria, a Micromonospora; an Ascomycota, Basidiomycota, or a Saccharomycetales; a Penicillium or a Trichocomaceae; a Spodoptera, a Spodoptera frugiperda, a Trichoplusia or a Trichoplusia ni; a Poaceae, a Triticum; an insect cell, optionally Sf9, a rabbit reticulocyte, Chinese hamster ovary (CHO), Human embryonic kidney (HEK) or a HeLa cell, a cultured human-derived cell; or, Leishmania tarentolae, Myxobacteria, Phellinus, Ceratocystis virescens, Cronartium fusiforme, Paenibacillus polymyxa, Mycolatopsis rifamycinica, Clostridium botulinum, Streptomyces verticillus, Marine bacteria, Archaea, Thermococcus S557, Methanocaldococcus jannaschii, Penicillium chrysogenum, Cephalosporium acremonium, Pleurotus ostreatus, Tolypocladium inflatum, Claviceps spp., Aspergillus alliaceus, Taxus brevifolia, Cephalotaxus harringtonii, Artemisia annua, Galanthus spp., Conus magus, Conus magus, Ecteinascidia turbinate, Discodermia dissoluta, Erythropodium caribaeorum or Bugula neritina; or, (e) at least one of the cytoplasmic extracts comprises an extract from or comprises an extract derived from an E. coli; and, at least one of the cytoplasmic extracts comprises an extract from or comprises an extract derived from an Actinomyces or a Streptomyces, and optionally the Actinomyces is: an Amycolatopsis, a Saccharopolyspora, a Streptomyces, Micromonospora; and optionally the Streptomyces is: S. coelicolor, S. albus, S. albus J1074, S. ambofaciens, S. ambofaciens BES2074, S. avermitilis, S. avermitilis SUKA17, S. coelicolor M1154, S. fradiae, S. roseosporus, S. toyocaensis, S. venezuelae, S. cinnamonensis, Streptomyces rapamycinicus, Streptomyces griseus, Streptomyces platensis, Streptomyces spheroides, and Streptomyces lividans; and optionally the Amycolatopsis is Amycolatopsis mediterranei, Amycolatopsis orientalis; and optionally the Saccharopolyspora is Saccharopolyspora erythraea, Saccharopolyspora spinosa; or, (f) the cells from which the at least one cytoplasmic or nuclear extract, or mixture thereof, has been derived, before isolation or harvesting of the extract, is: an activated or a stimulated cell; a cell exposed to chemical or a reagent in vitro; a genetically altered cell; a “strain engineered” cell (a cell modified by genetic strain engineering methods, e.g., including modification of cells from which the at least one cytoplasmic or nuclear extract has been derived); or, a cultured cell, wherein optionally the cells from which the at least one cytoplasmic or nuclear extract has been derived have had components of central metabolism (optionally redox metabolism, glycolysis, redox metabolism, pentose phosphate pathway, TCA cycle and amino acid biosynthesis), lipid or fatty acid biosynthesis, oxidative phosphorylation and/or protein synthesis upregulated, activated or co-activated, or de-activated, and optionally the cells from which the at least one cytoplasmic or nuclear extract has been derived are cultured under different environmental or in vitro culture conditions (optionally to turn on/off native enzymes, natural products (secondary metabolites) (NP) (including polyketides of class I, II or III, a non-ribosomal peptide or a hybrid polyketide-non ribosomal peptide), a natural product analog (NPA), or proteins; or for the extracts to have greater or fewer amounts of co-factors); and optionally the cells from which the at least one cytoplasmic or nuclear extract has been derived are in mid-log phase, interphase, mitotic (M) phase or cytokinesis, or undergoing mitosis, and optionally the strain engineering comprises ribosome and/or RNA polymerase engineering, optionally comprising: adding or making rpoB and rpsL mutants (components of ribosomal subunits) to enhance secondary metabolite production; mutations to the RNA polymerase machinery can be made to increase promoter binding affinity; deletion or overexpression of pathway or global regulators (activators and repressors); expressing mutant transcriptional regulators; and/or expressing or overexpressing ribosome recycling factor (RRF), and optionally the strain engineering comprises epigenetic modifications, optionally comprising phosphorylation, acetylation, methylation, ubiquitination, ADP-ribosylation, and/or glycosylation, and optionally the strain engineering comprises engineering in self-resistance, optionally by the upregulation or overexpression of resistance genes such as drrABC, avtAB and actAB, and optionally the strain engineering comprises genome-minimizing, optionally removing or disabling one, some, all or the majority of secondary metabolite biosynthetic gene clusters (SMBGCs), and optionally the strain engineering comprises combinatorial knockdown of secondary metabolite pathways, optionally by adding or expressing small RNAs targeting secondary metabolite biosynthesis, and optionally the strain engineering comprises expressing tRNAs for rare codons, optionally codons for AGA, AGG, AUA, CUA, GGA, CCC, and CGG, and optionally the strain engineering comprises over-expressing one or more chaperones native to strain, optionally a Streptomyces, optionally comprising over-expressing Hsp60, Hsp70, Hsp90, Hsp100, DnaK-DnaJ-GrpE and/or GroEL-GroES, e.g., to improve overall protein production, and optionally the strain engineering comprises inactivating RNaseE, optionally by mutation to enhance mRNA stability and consequently protein production, and optionally the strain engineering comprises expressing or overexpressing Streptomyces antibiotic regulatory protein (SARP) for positive regulation of antibiotic production, and optionally the strain engineering comprises expressing or overexpressing MbtH-like proteins for stimulating adenylation reactions, and optionally the strain engineering comprises expressing or overexpressing phosphopantetheinyl transferases (PPTases) proteins for stimulating post-translational modification of an apo-acyl carrier protein (apo-ACP) to activate polyketide synthases, and optionally the strain engineering comprises NAD(P)H regeneration, optionally expressing or overexpressing trans-hydrogenases for converting NADPH into NADH and NADPH+NAD+<=>NADH+NADP+; or, (g) the cells from which the at least one cytoplasmic or nuclear extract has been derived: or, (i) are free of or substantially free of cell wall, cell wall components, organelles or sub-cellular compartments; or (ii) are supplemented with: an organelle or sub-cellular compartment, wherein optionally the organelle comprises a natural or a synthetic Golgi organelle (optionally for glycosylation), a mitochondria or a chloroplast; a synthetic or a designer organelle, a synthetic nano- or micro-compartment, a synthetic or a designer micelle or liposome; an NAD(P)H or ATP recycling system; a mitochondria or mitochondrial extract; or a chaperone protein or a chaperone complex (optionally Hsp60, Hsp70, Hsp90, Hsp100, DnaK-DnaJ-GrpE and/or GroEL-GroES) or mbtH and its homologs, or a broad specificity 4′-Phosphopantetheine transferases or phosphoprotein phosphatase (PPTtase) including sfp and its homologs.

In alternative embodiments the products of manufacture further comprise additional ingredients, compositions or compounds, reagents, ions or element, buffers and/or solutions, wherein optionally the additional ingredients, compositions or compounds, reagents, ions or element, buffers and/or solutions are mixed into the extract or extracts, wherein optionally the additional ingredients, compositions or compounds, reagents, buffers and/or solutions comprise: nucleosides or nucleotides; lipids or fatty acids; carbohydrates, polysaccharides or sugars; nucleic acids or oligonucleotides; one or more enzymes, co-enzymes or enzyme co-factors; one or more amino acids; polycationic aliphatic amines or spermidine; a folinic acid, a 5-formyltetrahydrofolate or a leucovorin; a vitamin; a polyether or a polyethylene glycol (polyethylene oxide (PEO) or polyoxyethylene (POE)); a small-molecule redox reagent, an isopropyl β-D-1-thiogalactopyranoside (IPTG), a dithioerythritol (DTE) or a dithiothreitol (DTT); a glutamate or a glutamic acid; a carboxylic acid; an alpha-keto amino acid or a pyruvic acid; a regulator or activator of transcription or translation, or any combination thereof, wherein optionally the carbohydrate, polysaccharide or sugar comprises a maltodextrin, maltose, glucose and/or a hexametaphosphate (HMP); and optionally the co-enzyme or co-factor comprises an acyl-CoA precursor (optionally acetyl-CoA, malonyl-CoA, ethylmalonyl-CoA, methylmalonyl-CoA, isobutyryl-CoA, or propionyl-CoA), a nicotinamide adenine dinucleotide (NAD) or an NADH, a nicotinamide adenine dinucleotide phosphate (NADP) or an NADPH, a fluoromalonyl-CoA (F-CoA), or a S-Adenosyl methionine (SAM); and optionally the nucleosides or nucleotides comprise ATP, GTP, CTP, UTP or any combination thereof, and optionally the nucleic acids or oligonucleotides comprise transfer RNA (tRNA), small inhibitory RNA (siRNA), translational riboregulators or riboswitches, and wherein optionally the amino acid comprises non-natural amino acid (including those introduced using an “expanded genetic code”, as described by e.g., Malyshev et al., Nature 509:385, May 2014), and optionally the ion or element comprises an inorganic phosphate, a phosphonate, a phosphonic acid or a phosphonate salt, and optionally the one or more enzymes comprise an enzyme for modification of a product, a small molecule, a natural product (secondary metabolite) (NP) or a natural product analog (NPA), a protein, a lipid or fatty acid, a polysaccharide or a nucleic acid, and optionally the enzyme modification comprises: lipidation, pegylation, glycosylation, adding hydrophobic groups, myristoylation, palmitoylation, isoprenylation, prenylation, lipoylation, adding a flavin moiety (optionally comprising addition of: a flavin adenine dinucleotide (FAD) an FADH₂, a flavin mononucleotide (FMN), an FMNH₂), phospho-pantetheinylation, heme C addition, acylation, alkylation, butyrylation, carboxylation, malonylation, hydroxylation, adding a halide group, iodination, propionylation, S-glutathionylation, succinylation, glycation, adenylation, thiolation, condensation (optionally the “condensation” comprising addition of: an amino acid to an amino acid, an amino acid to a fatty acid, an amino acid to a sugar), or a combination thereof, and optionally the one or more enzymes comprise a CoA ligase, a phosphorylase, a glycosyl-transferase, a halogenase, a methyltransferase, a hydroxylase, a lambda phage GamS enzyme (optionally used with a bacterial or an E. coli extract, optionally at a concentration of about 3.5 mM), a Dsb (disulfide bond) family enzyme (optionally DsbA), or a combination thereof, or optionally the enzymes comprise one or more central metabolism enzyme (optionally tricarboxylic acid cycle (TCA, or Krebs cycle) enzymes, glycolysis enzymes or Pentose Phosphate Pathway enzymes), and optionally the additional ingredients, compositions or compounds, reagents, ions, buffers and/or solutions comprise protease, DNase or RNase inhibitors, a buffer, an anti-oxidant, a rare earths, a vitamin, a salt, a metal (optionally a trace metal, iron Fe, zinc Zn, Mg2+, Mn, vanadium) and/or a halogen, and optionally the additional ingredients, compositions or compounds, reagents, ions, buffers and/or solutions comprise a labeling agent (optionally a metabolic labeling agent), a detection or an affinity-tags, a fluorophore, reagents for biotinylation or biotin, a gold nanoparticle, an isotope or a radioactive isotope (optionally a metabolic labeling isotope, a ¹³C6-lysine, a ³H thymidine, a ³⁵S methionine, a ³²P orthophosphate, a ¹⁴C-labeled D-glucose); a photoreactive amino acid label (optionally a diazirine), a bioorthogonal labeling reagent (optionally an azide, an alkyne, an aldehyde or a ketone), an RNA polymerase, optionally a T7 or T3 RNA polymerase, optionally a purified polymerase, a sigma factor, optionally a Streptomyces sigma factor or, a sigma 70, a sigma 54, sigma factors HrdB, 19, 24, 28, 32 and/or 38), and optionally the additional ingredients, compositions or compounds, reagents, ions, buffers and/or solutions comprise one, several or all of the additional ingredients, compositions, compounds or reagents as set forth in Table 1, and optionally at the concentration set forth in Table 1.

In alternative embodiments of the products of manufacture: (a) the chemical, the natural product (NP), the NPA, or secondary metabolite is: a violacein, a butadiene, a propylene, a 1,4-butanediol, a 1,3-butanediol, a crotyl alcohol, a methyl vinyl carbinol, an isopropanol, an ethylene glycol, a terephthalic acid, an adipic acid, a hexamethylenediamine (HMDA), a caprolactam, a cyclohexanone, a aniline, a Methyl Ethyl Ketone (MEK), a fatty alcohol, an acrylic acid, an acrylate ester, a methyl methacrylate, a lipid, a carbohydrate, a beta-lactam, a polyketide, a macrolide, a macrolide having a 14-, 15- or 16-membered macrocyclic lactone ring, a trans-AT type I PKS, a Type II PKS, or a Type III PKS, a heterocyst glycolipid PKS-like, a cyclic peptide, a bottromycin or an antibiotic, or, (b) the chemical, the natural product (NP), the NPA, or secondary metabolite is a terpenoid, a steroid, an alkaloid, a fatty acid, a nonribosomal polypeptide (“NRP”; also referred to as “nonribosomal peptide”), an enzyme cofactor, an aminocoumarin, a melanin, an aminoglycosides/aminocyclitol, a microcin, an aryl polyene, a microviridin, a bacteriocin, a nucleoside, an oligosaccharide, a butyrolactone, a phenazine, a phosphoglycolipid, a cyanobactin, a phosphonate, a (dialkyl)resorcinol, a polyunsaturated fatty acid, an ectoine, a furan, a lycocin, a Head-to-tail cyclized peptide, a proteusin, a homoserine lactone, a sactipeptide, an indole, a siderophore, a ladderane lipid, a terpene, a lantipeptide, a thiopeptide, a linear azol(in)e-containing peptides (LAPs), a lasso peptide, a linaridin; and optionally the substantially isolated or a synthetic nucleic acid are in a linear or a circular form, and optionally the substantially isolated or a synthetic nucleic acid is contained in a circular or a linearized plasmid, vector or phage DNA, and optionally the substantially isolated or a synthetic nucleic acid comprises enzyme coding sequences operably linked to a homologous or a heterologous transcriptional regulatory sequence, optionally a transcriptional regulatory sequence is a promoter, an enhancer, or a terminator of transcription, and optionally the substantially isolated or a synthetic nucleic acid comprises has at least about 100, 200, 300, 400 or 500 or more base pair ends upstream of the promoter and/or downstream of the terminator; and optionally the promoter comprises a native promoter (a promoter used in an organism that is a source of the mixed extract) or a synthetic promoter, and optionally promoters operably linked to nucleic acids comprise a combination of promoters from all or several of the organisms that are the source of the mixed extract (optionally an E. coli and a Streptomyces extract, and a combination of E. coli and a Streptomyces promoter are used) (taking advantage of the available transcriptional machinery from E. coli and Streptomyces as well as a bacteriophage orthogonal RNA polymerase); and optionally the promoter is ermEp* (a heterologous promoter from Saccharopolyspora erythraea and is active in 20 E. coli), SF14p, or kasOp* (active in E. coli); and optionally all, or a subset, of the enzyme-encoding nucleic acid of the enzyme-encoding natural-product (NP), NPA, or a synthesizing operon or biosynthetic gene cluster are contained on separate linear nucleic acids (separate nucleic acid strands), optionally in equimolar concentrations in the mixed cytoplasmic or nuclear extract, and optionally, each separate linear nucleic acid comprises one, two, three, 4, 5, 6, 7, 8, 9, or 10 or more genes or enzyme-encoding sequences, and optionally the linear nucleic acid is present in a concentration of about 1.0 nM (nanomolar), 5 nM, 10 nM, 15 nM, 20 nM, 25 nM, 30 nM, 35 nM, nM, 45 nM or 50 nM or more or between about 1 nM and 100 nM; and optionally the enzyme-encoding nucleic acids, the linear nucleic acid or all, or a subset, of the enzyme-encoding nucleic acid of the enzyme-encoding natural-product synthesizing operon or biosynthetic gene cluster are immobilized, optionally immobilized on a bead or a chip; and optionally the enzyme encoded by the nucleic acid are between about 10 and 100 kDa, or about 10 kDa, 20 kDa, 30 kDa, 40 kDa, 50 kDa, 60 kDa, 70 kDa, 80 kDa, 90 kDa, or 100 kDa, 110 kDa, 120 kDa, 130 kDa, 140 kDa, 150 kDa, or more.

In alternative embodiments, provided are processes for in vitro, or cell free, transcription/translation, comprising: (a) providing a product of manufacture as described or provided herein; (b) incubating the product of manufacture or extracts thereof such that the substantially isolated or a synthetic nucleic acid comprising or encoding: the enzyme-encoding natural-product (NP)- or natural product analog (NPA)-synthesizing operon; the biosynthetic gene cluster, optionally the biosynthetic gene cluster comprising coding sequence for all or substantially all enzymes needed in the synthesis of a natural product (NP), NPA, or the secondary metabolite; the plurality of enzyme-encoding nucleic acids; or the plurality of enzyme-encoding nucleic acids for the at least two, several or all of the steps in the synthesis of a product or chemical or molecule, undergo coupled transcription and translation to synthesize a natural product (NP) or secondary metabolite, or a natural product analog (NPA) or secondary metabolite analog,
and optionally the substantially isolated or a synthetic nucleic acid comprises: (i) a genome, a gene or a DNA from a source other than the cell used for the extract (an exogenous nucleic acid), or an exogenous nucleic acid that has been engineered or mutated, optionally engineered or mutated in a protein coding region or in a non-coding region, (ii) a genome, a gene or a DNA from a cell used for the extract (an endogenous nucleic acid), or an endogenous nucleic acid that has been engineered or mutated, optionally engineered or mutated in a protein coding region or in a non-coding region, (iii) a genome, a gene or a DNA from one, both or several of the organisms used as a source for the extract, or, (iv) any or all of (i) to (iii).
and optionally the natural product (NP), natural product analog (NPA) or secondary metabolite is or comprises: a violacein, a butadiene, a propylene, a 1,4-butanediol, an isopropanol, an ethylene glycol, a terephthalic acid, an adipic acid, a hexamethylenediamine (HMDA), a caprolactam, a cyclohexanone, a aniline, a Methyl Ethyl Ketone (MEK), a fatty alcohol, an acrylic acid, an acrylate ester, a methyl methacrylate, a lipid, a carbohydrate, or an antibiotic, a butadiene, a propylene, a 1,4-butanediol, a 1,3-butanediol, a crotyl alcohol, a methyl vinyl carbinol, an isopropanol, an ethylene glycol, a terephthalic acid, an adipic acid, a hexamethylenediamine (HMDA), a caprolactam, a caprolactone, a hexanediol, a cyclohexanone, an aniline, a Methyl Ethyl Ketone (MEK), a fatty alcohol, an acrylic acid, an acrylate ester, a methyl methacrylate, a lipid, a carbohydrate, a beta-lactam, a polyketide, a macrolide, a macrolide having a 14-, 15- or 16-membered macrocyclic lactone ring, a ketolide, a taxane, a trans-AT type I PKS, a Type II PKS, or a Type III PKS, a heterocyst glycolipid PKS-like, a cyclic peptide, or a bottromycin, a terpenoid, a steroid, an alkaloid, a fatty acid, a nonribosomal polypeptide, an enzyme cofactor, an aminocoumarin, a melanin, an aminoglycosides/aminocyclitol, a microcin, an aryl polyene, a microviridin, a bacteriocin, a nucleoside, an oligosaccharide, a butyrolactone, a phenazine, a phosphoglycolipid, a cyanobactin, a phosphonate, a (dialkyl)resorcinol, a polyunsaturated fatty acid, an ectoine, a furan, a lycocin, a Head-to-tail cyclized peptide, a proteusin, a homoserine lactone, a sactipeptide, an indole, a siderophore, a ladderane lipid, a terpene, a lantipeptide, a thiopeptide, a linear azol(in)e-containing peptides (LAPs), a lasso peptide, a linaridin, a natural product (NP) or national product analog (NPA) useful for human and animal health and nutrition or crop health,
optionally comprising antibacterial agents, antifungal agents, cytotoxins, anticancer and antitumor agents, immunomodulators, anti-inflammatory, anti-arthritic, anthelminthic, insecticides, coccidiostats and anti-diarrhea agents, and also optionally comprising: a cytotoxin, an aminoglycoside antibiotic, a macrolide polyketide (Type I PKS), an oligopyrrole, a nonribosomal peptide, an aromatic polyketide (optionally an aromatic polyketide of a Type III PKS, an aromatic polyketide of Type II PKS), a complex isoprenoid, a beta-lactam, a terpenoid, a hybrid peptide-polyketide (from Type I PKS and NRPS), and/or a taxane, and also optionally comprising an antibacterial compound, optionally a vancomycin, erythromycin, daptomycin; antifungal agents (optionally amphotericin, nystatin); anticancer and antitumor agents for example doxorubicin, bleomycin; immunomodulators or immunosuppressants for example rapamycin, tacrolimus; anthelminthics for example avermectins; insecticides for example spinosyns; coccidiostats for example monensin, narasin; animal health compounds for example avilamycin, tilmicosin; optionally comprising acetogenins, actinorhodine, aflatoxin, albaflavenone, amphotericin, amphotericin b, annonacin, ansamycins, anthramycin, antihelminthics, avermectin, avilamycin, azithromycin, bleomycin, bullatacin, caprazamycins, carbomycin a, cephamycin c, cethromycin, chartreusin, calicheamicin, chloramphenicol, clarithromycin, clavulanate, coelchelin, cytotoxins, daptomycin, discodermolide, doxycycline, daunomycin, docetaxel, dolastatin, doxorubicin, echinomycin, endophenazine, epithienamycin, erythromycin, erythromycin a, fidaxomicin, FK506, flaviolin, fredericamycin, geldanamycin, ginsenoside compound K, Rh2, Rh1, Rg5, Rk1, Rg2, Rg3, Rg1, Rf, Re, Road, Rb2, Rc and Rb, geosmin, glucosyl-a47934, iso-migrastatin, ivermectin, josamycin, ketolides, kitasamycin, lovastatin, macbecin, macrolides, macrotetrolide, midecamycin, molvizarin, monensin, napyradiomycin, narasin, novobiocin, nystatin, oleandomycin, oxytetracycline, paclitaxel, pentalenolactone, phenalinolactione, pikromycin, pimaricin, pimecrolimus, polyene antimycotics, polyenes, polyketide macrolides, polyketides, radicicol, rapamycin, rifamycin, roxithromycin, sirolimus, solithromycin, spinosad, spinosyns, spiramycin, squamocin, staurosporine, streptomycin, tacrolimus, telithromycin, tetracenomycin, tetracyclines, teixobactin, thiocoraline, tilmicosin, troleandomycin, tylocine, tylosin, undecylprodigiosin, usnic acid, uvaricin, vancomycin and analogs thereof.

In alternative embodiments of the processes in vitro, or cell free, transcription/translation, at least one natural product or natural product analog, or structural analog of the secondary metabolite, is fused or conjugated to a carrier molecule, optionally a pharmaceutically acceptable carrier molecule, optionally a polymer, a protein or peptide, an antibody or fragment thereof, an affibody, a PEG or a PEG derivative, a lipophilic carrier including a fatty acid, optionally palmitoyl, myristoyl, stearic acid, 3-pentadecylglutaric acid, that associates with a serum protein such as albumin, LDL or HDL, and wherein optionally the carrier increases blood circulation time or cell-targeting or both for the NP or NPA, and optionally the natural product (NP) or NPA, or structural analog of the secondary metabolite: is fused or conjugated to the carrier in the extract, and optionally is enriched before being fused or conjugated to the carrier, or is isolated before being fused or conjugated to the carrier, and optionally the NP or NP, or structural analog of the secondary metabolite, is site-specifically fused or conjugated to the carrier, optionally wherein the NP or NPA, or structural analog of the secondary metabolite, is modified to comprise a group capable of the site-specific fusion or conjugation to the carrier, optionally where the NP or NPA, or structural analog of the secondary metabolite, is synthesized in the extract to comprise the site-specific reactive group, and, optionally wherein the library contains a plurality of NP or NPA, or structural analogs of the secondary metabolite, each having a site-specific reactive group at a different location on the NP or NPA, or structural analog of the secondary metabolite, and optionally the site-specific reactive group can react with a cysteine or lysine or glutamic acid on the carrier.

In alternative embodiments, provided are methods for screening for: a modulator of protein activity, transcription or translation or cell function; a toxic metabolite or a protein; a cellular toxin; an inhibitor of transcription or translation, comprising: (a) providing a product of manufacture as described or provided herein, wherein the product of manufacture comprises at least one protein-encoding nucleic acid; (b) providing a test compound; (c) combining or mixing the test compound with the product of manufacture under conditions wherein the extract initiates or completes transcription and/or translation, or modifies a molecule (optionally a protein, a small molecule, a natural product (NP), natural product analog (NPA) or secondary metabolite or a lipid) and, (d) determining or measuring any change in the functioning or products of the extract, or the transcription and/or translation, wherein determining or measuring a change in the protein activity, transcription or translation or cell function identifies the test compound as a modulator of that protein activity, transcription or translation or cell function.

In alternative embodiments provided are in vitro methods for making, synthesizing or altering the structure of a compound, composition, organic molecule small molecule or natural product (NP), natural product analog (NPA) or secondary metabolite or library thereof comprising using a product of manufacture as provided herein, or by using a process or method as provided herein. In alternative embodiments, at least two or more of the altered compounds are synthesized to create a library of altered compounds, and optionally the library is a natural product analog library.

In alternative embodiments, provided are libraries of: natural products (NPs) or natural product analogs (NPAs), or structural analogs of a secondary metabolite, or a combination thereof, prepared, synthesized or modified by a method comprising use of a product of manufacture as provided herein, or by using a process or method as provided herein. In alternative embodiments, the method for preparing, synthesizing or modifying the natural products or natural product analogs, or structural analogs of the secondary metabolite, or the combination thereof, comprises using an extract from an Escherichia and from an Actinomyces, optionally a Streptomyces. In alternative embodiments, at least one natural product or natural product analog, or structural analog of the secondary metabolite, is fused or conjugated to a carrier molecule, optionally a pharmaceutically acceptable carrier molecule, optionally a polymer, a protein or peptide, an antibody or fragment thereof, an affibody, a PEG or a PEG derivative, a lipophilic carrier including a fatty acid, optionally palmitoyl, myristoyl, stearic acid, 3-pentadecylglutaric acid, that associates with a serum protein such as albumin, LDL or HDL, and wherein optionally the carrier increases blood circulation time or cell-targeting or both for the NP or NPA. In alternative embodiments, the natural product (NP) or NPA, or structural analog of the secondary metabolite: is fused or conjugated to the carrier in the extract, and optionally is enriched before being fused or conjugated to the carrier, or is isolated before being fused or conjugated to the carrier.

In alternative embodiments of the libraries: the NP or NP, or structural analog of the secondary metabolite, is site-specifically fused or conjugated to the carrier; optionally wherein the NP or NPA, or structural analog of the secondary metabolite, is modified to comprise a group capable of the site-specific fusion or conjugation to the carrier, optionally where the NP or NPA, or structural analog of the secondary metabolite, is synthesized in the extract to comprise the site-specific reactive group, and optionally wherein the library contains a plurality of NP or NPA, or structural analogs of the secondary metabolite, each having a site-specific reactive group at a different location on the NP or NPA, or structural analog of the secondary metabolite, and optionally the site-specific reactive group can react with a cysteine or lysine or glutamic acid on the carrier.

In alternative embodiments of the libraries: the natural product analogs (NPAs) or structural analogs of the secondary metabolite, or the diversity of natural product analogs (NPAs) or structural analogs of the secondary metabolite, is generated by a process comprising modifying the natural product (NP) or secondary metabolite chemically or by enzyme modification, wherein optionally the enzyme modification comprises modification of the natural product (NP) or structural analog of the secondary metabolite by: halogenation, lipidation, pegylation, glycosylation, adding hydrophobic groups, myristoylation, palmitoylation, isoprenylation, prenylation, lipoylation, adding a flavin moiety (optionally comprising addition of: a flavin adenine dinucleotide (FAD) an FADH₂, a flavin mononucleotide (FMN), an FMNH₂), phospho-pantetheinylation, heme C addition, acylation, alkylation, butyrylation, carboxylation, malonylation, hydroxylation, adding a halide group, iodination, propionylation, S-glutathionylation, succinylation, glycation, adenylation, thiolation, condensation (optionally the “condensation” comprising addition of: an amino acid to an amino acid, an amino acid to a fatty acid, an amino acid to a sugar), or a combination thereof, and optionally the enzyme modification comprises modification of the natural product (NP) or structural analog of the secondary metabolite by one or more enzymes comprising: a CoA ligase, a phosphorylase, a glycosyl-transferase, a halogenase, a methyltransferase, a hydroxylase, a lambda phage GamS enzyme (optionally used with a bacterial or an E. coli extract, optionally at a concentration of about 3.5 mM), a Dsb (disulfide bond) family enzyme (optionally DsbA), or a combination thereof, or optionally the enzymes comprise one or more central metabolism enzyme (optionally tricarboxylic acid cycle (TCA, or Krebs cycle) enzymes, glycolysis enzymes or Pentose Phosphate Pathway enzymes), and optionally the chemical or enzyme modification comprises addition, deletion or replacement of a substituent or functional groups, optionally a hydroxyl group, an amino group, a halogen, an alkyl or a cycloalkyl group, optionally by hydration, hydrogenation, an Aldol condensation reaction, condensation polymerization, halogenation, oxidation, dehydrogenation, or creating one or more double bonds.

In alternative embodiments, provided are compositions comprising: a natural product (NP) or natural product analog (NPA) or structural analog of the secondary metabolite, obtained from a library as provided herein, wherein optionally the composition further comprises, is formulated with, or is contained in: a liquid, a solvent, a solid, a powder, a bulking agent, a filler, a polymeric carrier or stabilizing agent, a liposome, a particle or a nanoparticle, a buffer, a carrier, a delivery vehicle, or an excipient, optionally a pharmaceutically acceptable excipient. In alternative embodiments, of the compositions, the natural product (NP) or natural product analog (NPA) is fused or conjugated to a carrier molecule, optionally a pharmaceutically acceptable carrier molecule, optionally a polymer, a protein or peptide, an antibody or fragment thereof, an affibody, a PEG or a PEG derivative, a lipophilic carrier including a fatty acid, optionally palmitoyl, myristoyl, stearic acid, 3-pentadecylglutaric acid, that associates with a serum protein such as albumin, LDL or HDL, and wherein optionally the carrier increases blood circulation time or cell-targeting or both for the NP or NPA. In alternative embodiments, the natural product or NPA is fused or conjugated to the carrier in the extract, and optionally is enriched before being fused or conjugated to the carrier, or is isolated before being fused or conjugated to the carrier. In alternative embodiments, the NP or NP is site-specifically fused or conjugated to the carrier, optionally wherein the NP or NPA is modified to comprise a group capable of the site-specific fusion or conjugation to the carrier, optionally where the NP or NPA is synthesized in the extract to comprise the site-specific reactive group, and optionally wherein the library contains a plurality of NP or NPA each having a site-specific reactive group at a different location on the NP or NPA, and optionally the site-specific reactive group can react with a cysteine or lysine or glutamic acid on the carrier.

In alternative embodiments, provided are compositions or methods according to any embodiment of the invention, substantially as herein before described, or described herein, with reference to any one of the examples.

The details of one or more exemplary embodiments as provided herein are set forth in the accompanying drawings and the description below. Other features, objects, and advantages of the invention will be apparent from the description and drawings, and from the claims.

All publications, patents and patent applications cited herein are hereby expressly incorporated by reference for all purposes.

The embodiments of the description described herein are not intended to be exhaustive or to limit the disclosure to the precise forms disclosed in the following drawings or detailed description. Rather, the embodiments are chosen and described so that others skilled in the art can appreciate and understand the principles and practices of the description.

### BRIEF DESCRIPTION OF THE DRAWINGS

The drawings set forth herein are illustrative of embodiments of the invention and are not meant to limit the scope of the invention as encompassed by the claims.

FIG. 1 schematically illustrates an exemplary cell-free transcription-translation system (“TX-TL”) as provided herein, comprising a rapid prototyping platform for the synthesis and identification of natural products (NPs) or natural product analogs (NPAs) from biosynthetic gene cluster pipelines and modified and heterologous extracts; and, the combinatorial biosynthesis of natural products and natural product analogs, as discussed in detail, below.

FIG. 2 schematically illustrates that an exemplary cell-free transcription-translation system as provided herein can be used as a rapid expression screening platform; in this example, the expression of the 1,4-BDO (1,4-butanediol) pathway genes were validated in the exemplary TX-TL using linear DNA. Exemplary TX-TL protocol validated by using a 1,4-BDO pathway expression system using linear DNA to synthesize 1,4-BDO; this exemplary system validated expression of 4 out of 5 BDO pathway genes (expression of Cat2 not detected, as expected; not detected in in vivo experiments via SDS-PAGE).

FIG. 3 schematically illustrates an exemplary cell-free transcription-translation system as provided herein, and graphically illustrates data validating use of linear DNA in an exemplary TX-TL for 1,4-BDO production; 1,4-BDO production was validated in wild-type (WT) (BL21) and advanced strain extracts (advanced 1,4-BDO production strain) with non- and engineered pathway enzymes. An increase in 1,4-BDO was observed for all 1,4-BDO producing strain extracts versus control (left and right panels) as well as for a strain having an engineered 1,4-BDO pathway versus an extract from a wild type (WT) strain having a 1,4-BDO pathway (left panel).

FIG. 4 schematically illustrates an exemplary cell-free transcription-translation system as provided herein, and graphically illustrates data validating use of the exemplary TX-TL for 1,4-BDO production, and that TX-TL can be used for the rapid prototyping of multiple pathways, and illustrates an exemplary TX-TL protocol for synthesizing 1,3-butanediol from biomass sugars (right panel) that enables dramatic shortening of research and development time (left panel).

FIG. 5 schematically illustrates an exemplary cell-free transcription-translation system as provided herein, and graphically illustrates data validating use of the exemplary TX-TL for 1,4-BDO production, and that TX-TL can be used for optimization of the structure or synthesis of any product, e.g., a specialty chemical or a small molecule chemical, or a natural product, e.g., 1,4-BDO, and identification of synthetic pathway bottlenecks (and resolution of the bottlenecks), exemplified herein by ADH (alcohol dehydrogenase) or ALD (aldehyde dehydrogenase) steps in a 1,4-BDO pathway.

FIG. 6 schematically illustrates an exemplary TX-TL system as provided herein that synthesizes a combinatorial library of polyketides using 6-deoxyerythronolide B synthase (DEBS), as described in detail in Example 3, below.

FIG. 7 schematically and graphically illustrate an exemplary TX-TL protocol, comprising a 1 week process for preparation of each batch of TX-TL buffer/extract, produces extracts to conduct about 750 reactions, which reduces the amount of resources, personnel and cost compared to a fully in vivo process.

FIG. 8 schematically illustrates an exemplary TX-TL system as provided herein for the application and development of TX-TL for industrial metabolic engineering.

FIG. 9 depicts an exemplary TX-TL system as provided herein where cell extracts are modified either by exogenous compound addition or via cell genetic engineering to create diversity. The system as provided is automatable.

FIG. 10A-B depicts an exemplary TX-TL system as provided herein where cell extracts from more than one cell are combined (FIG. 10B) along with refactoring of an exemplary natural product target, actinorhodine, gene cluster as illustrated in FIGS. 10A and 10B.

FIG. 11 depicts the various aspects of natural product (NP) and NP analog development and screening that can be addressed by or applied in the TX-TL system and methods as provided herein.

FIG. 12 describes an exemplary natural product target teixobactin (TXB) for optimizing production or for generating its natural product analogs using the TX-TL systems and methods provided herein.

FIG. 13 depicts a teixobactin gene cluster that can be generated for use in the TX-TL system and methods described herein.

FIG. 14 depicts a central metabolism amino acid precursors used by the gene cluster enzymes for teixobactin.

FIG. 15 depicts a process for developing a natural product host strain (teixobactin is modeled) where highlighted steps (underlined) can be facilitated using TX-TL as described herein to generate a host cell improved in TXB production or production of its natural product analogs.

Like reference symbols in the various drawings indicate like elements, unless otherwise stated.

Reference will now be made in detail to various exemplary embodiments of the invention, examples of which are illustrated in the accompanying drawings. The following detailed description is provided to give the reader a better understanding of certain details of aspects and embodiments of the invention, and should not be interpreted as a limitation on the scope of the invention.

### DETAILED DESCRIPTION

Methods and systems are provided herein to facilitate research, development and production of new compounds, especially complex compounds such as natural products and natural product and secondary metabolite analogs.

The methods and systems provided herein are cell-free systems that serve as a platform to emulate in vivo cellular environments and provide a valuable platform for understanding and expanding the capabilities of natural systems. Cell-free systems provided herein have numerous applications for industrial metabolic engineering by allowing rapid expression and activity screening without the need for plasmid based cloning and in vivo propagation, enabling rapid process/product pipelines. The lack of a cell wall or dependence on cellular viability allows for a flexible platform for constructing and characterizing complex biochemical systems and pathways. The lack of a cell wall also provides for the ability to easily screen toxic metabolites and proteins.

A key feature of the methods and systems provide herein is that biosynthesis pathway flux to a target compound can be optimized by directing resources to user defined objectives and consequently allows for the exploration of a large sequence space. Central metabolism, oxidative phosphorylation, and protein synthesis can be co-activated by the user.

The methods and systems provided herein exploit and extend known simple cell-free systems developed for several organisms including E. coli, yeast, insect, wheat germ, rabbit and human. E. coli based TX-TL cell-free expression systems have been used to produce equivalents amounts of at least a single protein as similar commercial in vivo protein-production systems.

The TX-TL methods and systems provided herein can be used to rapidly prototype novel complex biocircuits as well as metabolic pathways. Protein expression from multiple DNA pieces, including linear and plasmid based DNA, can be performed. The methods and systems provided herein enable modulating concentrations of DNA encoding individual pathway enzymes and testing the related effect on metabolite production. The ability to express multi-enzyme pathways using linear DNA in the methods and systems provided herein bypasses the need for in vivo selection and propagation of plasmids. Linear DNA fragments can be assembled in 1 to 3 hours (hrs) via isothermal or Golden Gate assembly techniques and be immediately used for a TX-TL reaction. The TX-TL reaction can take place in several hours, e.g. approximately 8 hours. The use of linear DNA provides a valuable platform for rapid prototyping libraries of DNA. In the methods and systems provided herein mechanisms of regulation and transcription exogenous to E. coli, such as the tet repressor and T7 RNA polymerase, or other host cell extracts, can be supplemented as defined by the user to generate and maximize endogenous properties, diversity or production. The methods and systems provide herein further enhance diversity and production of target compounds by modifying endogenous properties including mRNA and DNA degradation rates. ATP regeneration systems that allow for the recycling of inorganic phosphate, a strong inhibitor of protein synthesis, are manipulated in the methods and systems provided herein. Redox potential, including e.g., NAD/NADH, NADP/NADPH, are regenerated in TX-TL, and methods for modifying redox and availability of specific cofactors which in turn enables the user to selectively modulate any reaction in the TX-TL system are provided herein.

In alternative embodiments, provided are cell-free transcription-translation systems (TX-TL) that function as rapid prototyping platforms for the synthesis, modification and identification of products, e.g., natural products (NPs) or natural product analogs (NPAs), from biosynthetic gene cluster pipelines and natural product gene clusters. In alternative embodiments, exemplary TX-TL systems as provided herein are used for the combinatorial biosynthesis of natural products and natural product analogs and secondary metabolites. In alternative embodiments, exemplary TX-TL systems as provided herein are used for the rapid prototyping of complex biosynthetic pathways as a way to rapidly assess combinatorial designs before moving to cellular hosts. In alternative embodiments, these exemplary TX-TL systems are multiplexed for high-throughput automation, thus, provided are TX-TL platforms for rapid prototyping of natural product gene clusters and the natural products they encode and synthesize.

In alternative embodiments, provided are natural products (NPs) (secondary metabolites) and natural product analogs (NPAs), and libraries of these NPs and NPAs, and products of manufacture (including e.g., mixtures of cytoplasmic extracts) and processes for producing these NPs and NPAs and NP- and NPA-comprising libraries, and for increasing, modifying and improving the synthesis of these NPs and NPAs. In alternative embodiments, provided are gene pathways such as gene clusters or operons that are combinatorially altered as provided herein to create natural product analog libraries. In alternative embodiments, NPAs as provided herein, or NPs and NPAs made using processes as provided herein, including NPs and NPAs from NP- and NPA-comprising libraries provided herein, are useful for e.g., human and animal health and nutrition, industrial and agricultural uses, e.g., for crop health; for example, any of these NPs and NPAs can comprise: antibacterial agents, antifungal agents, cytotoxins, anticancer and antitumor agents, immunomodulators, anti-inflammatory, anti-arthritic, anthelminthic, insecticides, coccidiostats and anti-diarrhea agents. Examples of these and other natural products whose production can be increased and improved by the present invention or that can provide a template gene pathway (cluster, operon) amenable to the combinatorial aspects of the invention to create natural product analog libraries include: cytotoxins, macrolide polyketides (Type I PKS), oligopyrroles, nonribosomal peptides, aromatic polyketides, aromatic polyketides of Type III PKS, aromatic polyketides of Type II PKS, complex isoprenoids, beta-lactams, terpenoids, and Hybrid peptide-polyketides (from Type I PKS and NRPS), and includes those that are antibacterial compounds, for example vancomycin, erythromycin, daptomycin; antifungal agents for example amphotericin, nystatin; anticancer and antitumor agents for example doxorubicin, bleomycin; immunomodulators or immunosuppressants for example rapamycin, tacrolimus; anthelminthics for example avermectins; insecticides for example spinosyns; coccidiostats for example monensin, narasin; animal health compounds for example avilamycin, tilmicosin; and other compounds including acetogenins, actinorhodine, aflatoxin, albaflavenone, amphotericin, amphotericin b, annonacin, ansamycins, anthramycin, antihelminthics, avermectin, avilamycin, azithromycin, bleomycin, bullatacin, caprazamycins, carbomycin a, cephamycin c, cethromycin, chartreusin, calicheamicin, chloramphenicol, clarithromycin, clavulanate, coelchelin, cytotoxins, daptomycin, discodermolide, doxycycline, daunomycin, docetaxel, dolastatin, doxorubicin, echinomycin, endophenazine, epithienamycin, erythromycin, erythromycin a, fidaxomicin, FK506, flaviolin, fredericamycin, geldanamycin, ginsenoside compound K, Rh2, Rh1, Rg5, Rk1, Rg2, Rg3, Rg1, Rf, Re, Road, Rb2, Re and Rb, geosmin, glucosyl-a47934, iso-migrastatin, ivermectin, josamycin, ketolides, kitasamycin, lovastatin, macbecin, macrolides, macrotetrolide, midecamycin, molvizarin, monensin, napyradiomycin, narasin, novobiocin, nystatin, oleandomycin, oxytetracycline, paclitaxel, pentalenolactone, phenalinolactione, pikromycin, pimaricin, pimecrolimus, polyene antimycotics, polyenes, polyketide macrolides, polyketides, radicicol, rapamycin, rifamycin, roxithromycin, sirolimus, solithromycin, spinosad, spinosyns, spiramycin, squamocin, staurosporine, streptomycin, tacrolimus, telithromycin, tetracenomycin, tetracyclines, teixobactin, thiocoraline, tilmicosin, troleandomycin, tylocine, tylosin, undecylprodigiosin, usnic acid, uvaricin, vancomycin and analogs thereof.

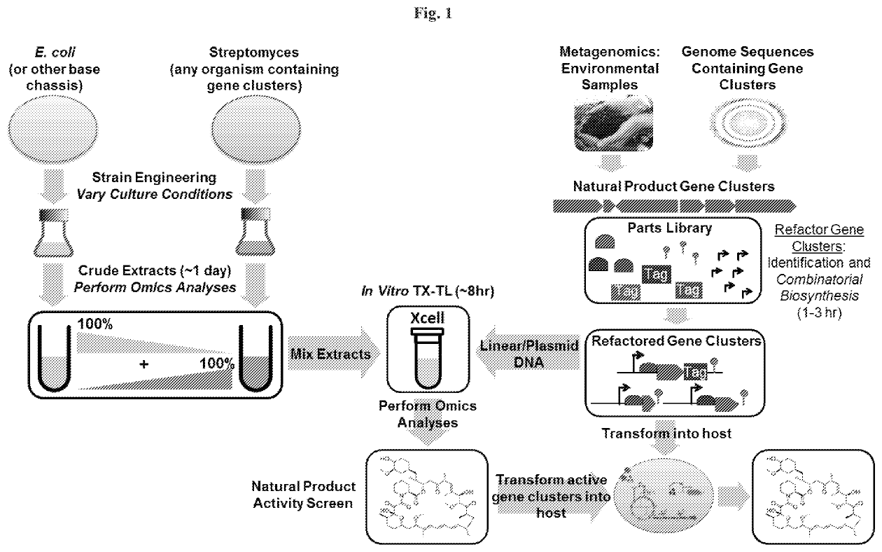

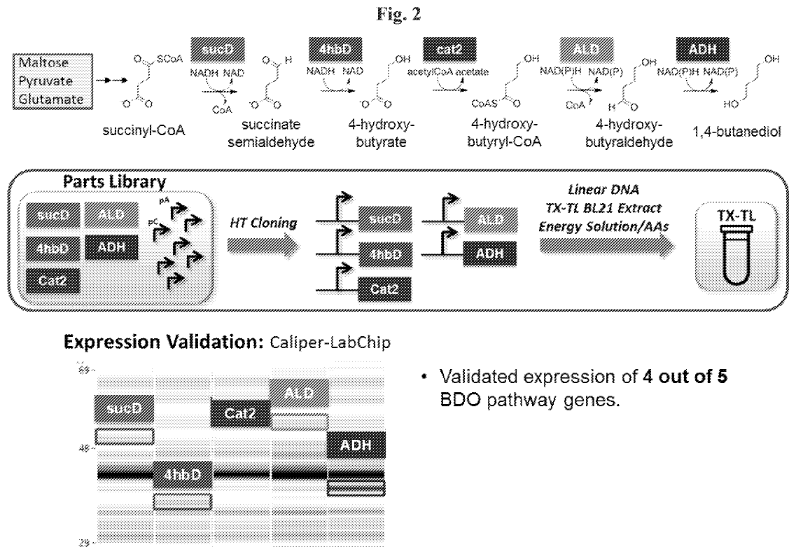

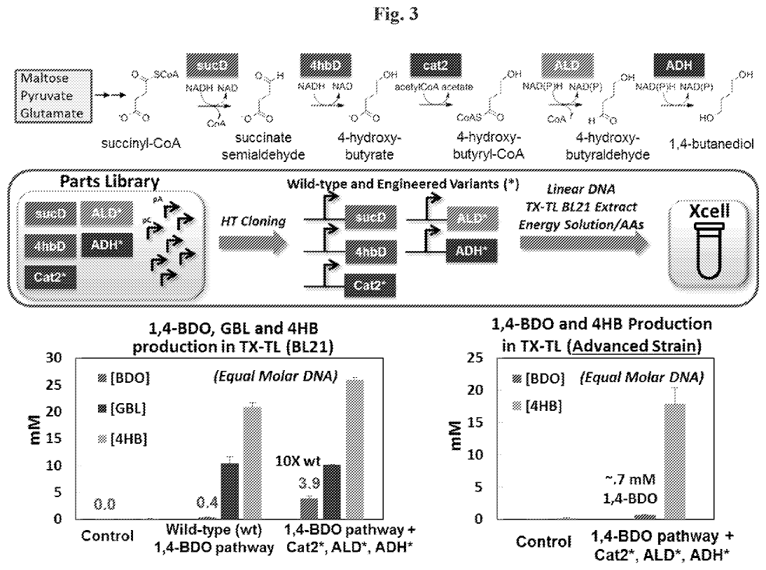

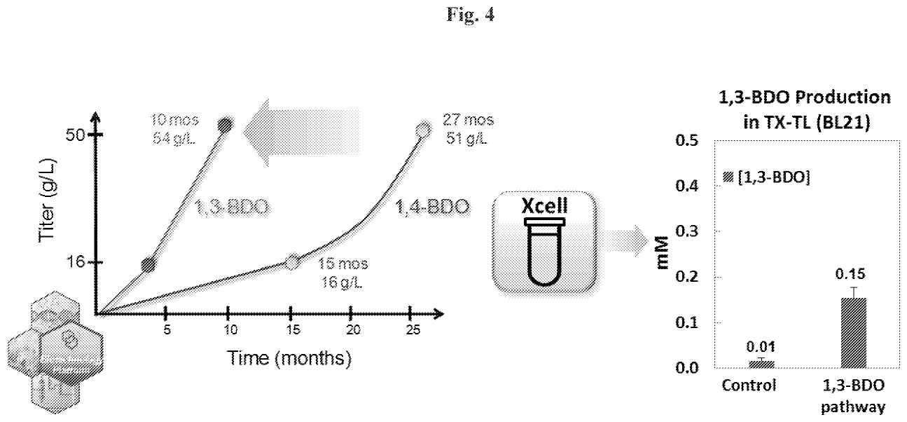

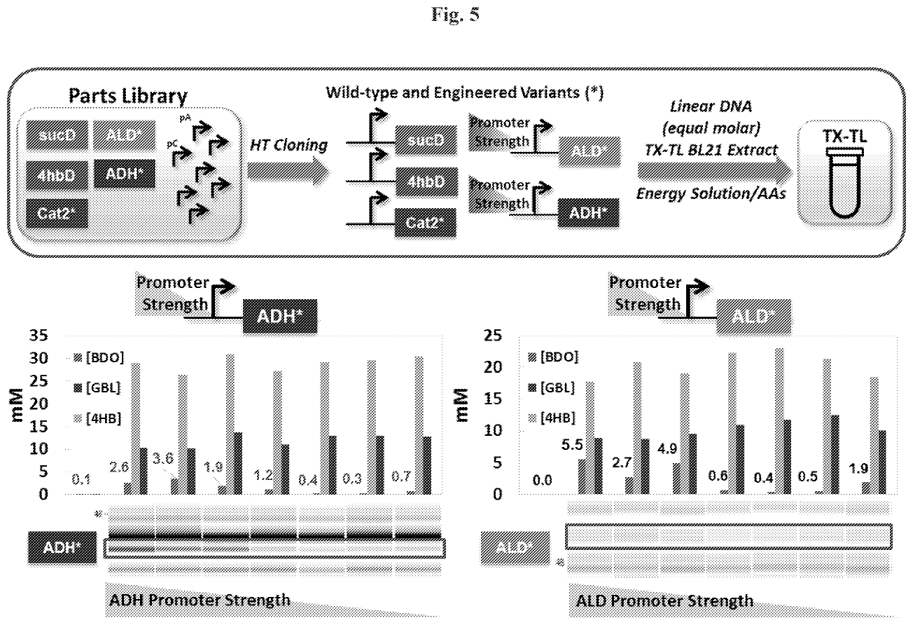

In alternative embodiments, TX-TL provided herein are used for as rapid prototyping platforms for the synthesis, modification and identification of natural products (NPs) or natural product analogs (NPAs) from biosynthetic gene cluster pipelines; and, for the combinatorial biosynthesis of natural products and natural product analogs, as illustrated in FIG. 1. In alternative embodiments, TX-TL provided herein are used as rapid expression screening platforms, e.g., for the expression of genes encoding for the production of natural products (NPs) or natural product analogs (NPAs), e.g., using the exemplary 1,4-BDO pathway genes, as validated using linear DNA, as illustrated in FIG. 2. TX-TL systems as provided herein have been validated using of the exemplary TX-TL for 1,4-BDO production in wild-type (BL21) and advanced strain extracts (advanced 1,4-BDO production strain) with non- and engineered pathway enzymes, as illustrated in FIG. 3. In alternative embodiments, TX-TL provided herein can be used for the rapid prototyping of multiple pathways, as illustrated in FIG. 4. In alternative embodiments, TX-TL provided herein are used for the optimization of natural product synthetic pathways, e.g., 1,4-BDO, production, and for the identification of synthetic pathway bottlenecks, as illustrated in FIG. 5.

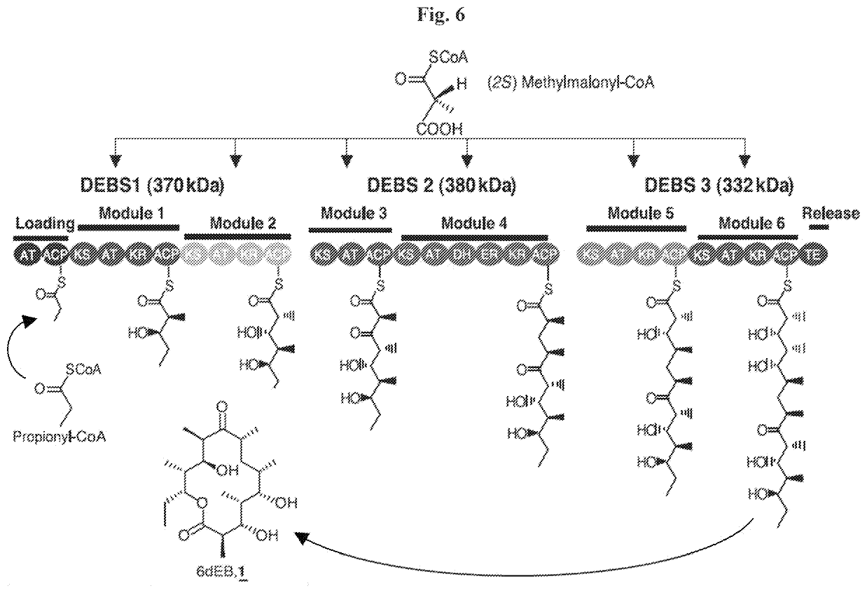

In alternative embodiments, TX-TL provided herein are used for the rapid identification and combinatorial biosynthesis of natural products (NPs) or natural product analogs (NPAs), including those made from complex gene clusters e.g., as shown in FIG. 6 for one natural product. An exemplary feature of this exemplary platform is that an unprecedented level of chemical diversity of natural products can be created and explored. In alternative embodiments, combinatorial biosynthesis approaches are executed through the modification of strains and culture conditions, refactored gene cluster combinations and by combining both methods.

In alternative embodiments, TX-TL systems and processes provided herein are cell-free TX-TL platforms that can use either or a mixture of: single organism crude extracts; synthetic cytoplasmic or nuclear extracts; or, mixtures of crude or processed (modified) cytoplasmic or nuclear extracts from a “base” organism (or “base chassis”), such as E. coli or Saccharomyces cerevisiae (S. cerevisiae), and (as an at least second extract) from an organism containing natural product biosynthetic gene clusters, such as an organism from the Actinomyces genus, e.g., a Streptomyces. The term “base” organism or “base chassis” refers to an organism which has shown to be a robust host for cell-free transcription-translation systems (TX-TL); as well as being a suitable heterologous expression host. In alternative embodiments, strain engineering approaches as well as modification of the growth conditions are used (on the organism from which an at least one extract is derived) towards the creation of extracts as provided herein, including crude or modified extracts, to generate mixed extracts (of the products of manufacture as provided herein) with varying proteomic and metabolic capabilities in the final TX-TL reaction. In alternative embodiments, both approaches are used to tailor or design a final TX-TL reaction system for the purpose of the identification and/or synthesis of natural products, or for the creation of natural product analogs through combinatorial biosynthesis approaches.

In alternative embodiments, in the preparation and characterization of the mixed extracts of the products of manufacture as provided herein, on either initial crude extracts and/or the “final” mixed product, proteomic approaches are conducted to assess and quantify the proteome available for the final TX-TL reactions. In addition, in alternative embodiments, ¹³C metabolic flux analysis (MIFA) and/or metabolomics studies are conducted in TX-TL reactions to create a flux map associated with a “starting” extract, e.g., a crude extract, or the “final” mixed extract product of manufacture, to characterize the resulting metabolome of the extract or extracts.

In alternative embodiments, provided are products of manufacture and processes for addressing one of the primary challenges in natural product discovery—that many predicted gene clusters cannot be expressed under laboratory conditions in the native host, or when cloned into a heterologous host. In alternative embodiments, provided are in vitro transcription/translation (TX-Tl) systems, including products of manufacture and processes that express novel gene clusters without the regulatory constraints of the cell. In alternative embodiments, some or all of the genes are refactored into operons to remove native transcriptional and translational regulation. In alternative embodiments the TX-TL reaction is performed using: one or a combination of extracts from various “chassis” organisms, such as E. coli, and one or a combination of second species, e.g., related to a native organism, e.g., an organism that synthesizes the natural product of interest to be synthesized using products of manufacture and processes provided herein. This can give the advantage of a robust transcription/translation machinery, combined with any unknown components of the native species that might be needed for proper protein folding or activity, or to supply precursors for the natural product pathway. In alternative embodiments, if these factors are known they can be expressed in the chassis organism prior to making the cell-free extract or the purified components added directly to the extract.

Combinatorial TX-TL as provided herein can be used to produce libraries of new compounds, including natural product (NP) libraries. For example, an exemplary refactored NP pathway can vary enzyme specificity at any step to introduce new functional groups and analogs at any one or more sites in a compound or NP. Exemplary processes can vary enzyme specificity to allow only one functional group in a mixture to pass to the next step, thus allowing each reaction mixture to generate a specific NP analog. Exemplary processes can vary the availability of functional groups at any step to control which group or groups are added at that step. Exemplary processes can vary a domain of an enzyme to modify its specificity and analog created. Exemplary processes can add a domain of an enzyme or an entire enzyme module to add novel chemical reaction steps to the NP pathway.

### Nucleic Acids for TX-TL Expression Systems

In alternative embodiments, products of manufacture as provided herein, including cytoplasmic or nuclear extracts, comprise use of nucleic acids which can be substantially isolated or a synthetic nucleic acids comprising or encoding: an enzyme-encoding natural-product synthesizing operon; a biosynthetic gene cluster, optionally a biosynthetic gene cluster comprising coding sequence for all or substantially all enzymes needed in the synthesis of a natural product; a plurality of enzyme-encoding nucleic acids; a plurality of enzyme-encoding nucleic acids for at least two, several or all of the steps in the synthesis of a product or chemical or molecule, wherein optionally the product or chemical is a natural product (NP) or natural product analogs (NPA). In alternative embodiments, the substantially isolated or a synthetic nucleic acids are in a linear or a circular form, or are contained in a circular or a linearized plasmid, vector or phage DNA. In alternative embodiments, the substantially isolated or a synthetic nucleic acid comprises enzyme coding sequences operably linked to a homologous or a heterologous transcriptional regulatory sequence, optionally a transcriptional regulatory sequence is a promoter, an enhancer, or a terminator of transcription. In alternative embodiments, the substantially isolated or a synthetic nucleic acids comprise at least about 50, 100, 200, 250, 300, 350, 400, 450, 500, 550, 600 or more base pair ends upstream of the promoter and/or downstream of the terminator.

In alternative embodiments, expression constructs, vehicles or vectors are provided to make, or to include, or contain within, one or more nucleic acids used in products of manufacture (e.g., cytoplasmic or nuclear extracts) and processes provided herein. In alternative embodiments, nucleic acids used in products of manufacture (e.g., cytoplasmic or nuclear extracts) and processes are operably linked to an expression (e.g., transcription or translational) control sequence, e.g., a promoter or enhancer, e.g., a control sequence functional in a cell from which an extract has been derived. In alternative embodiments, expression constructs, expression vehicles or vectors, plasmids, phage vectors, viral vectors or recombinant viruses, episomes and artificial chromosomes, including vectors and selection sequences or markers containing nucleic acids are used to make or express the products of manufacture as provided herein. In alternative embodiments, the expression vectors also include one or more selectable marker genes and appropriate expression control sequences.

Selectable marker genes also can be included that, for example, provide resistance to antibiotics or toxins, complement auxotrophic deficiencies, or supply critical nutrients not in an extract. Expression control sequences can include constitutive and inducible promoters, transcription enhancers, transcription terminators, and the like which are well known in the art. When two or more exogenous encoding nucleic acids are to be co-expressed, both nucleic acids can be inserted, for example, into a single expression vehicle (e.g., a vector) or in separate expression vehicles. For single vehicle/vector expression, the encoding nucleic acids can be operationally linked to one common expression control sequence or linked to different expression control sequences, such as one inducible promoter and one constitutive promoter.

In alternative embodiments, nucleic acid analysis such as Northern blots or polymerase chain reaction (PCR) amplification of mRNA, or immunoblotting, are used for analysis of expression of gene products, e.g., enzyme-encoding message; any analytical method can be used to test the expression of an introduced nucleic acid sequence or its corresponding gene product. The exogenous nucleic acid can be expressed in a sufficient amount to produce the desired product, and expression levels can be optimized to obtain sufficient expression.

In alternative embodiments, multiple enzyme-encoding nucleic acids (e.g., two or more genes) are fabricated on one polycistronic nucleic acid. In alternative embodiments, one or more enzyme-coding nucleic acids of a desired synthetic pathway are fabricated on one linear or circular DNA. In alternative embodiments, all or a subset of the enzyme-encoding nucleic acid of an enzyme-encoding natural-product synthesizing operon or biosynthetic gene cluster are contained on separate linear nucleic acids (separate nucleic acid strands), optionally in equimolar concentrations in the mixed cytoplasmic or nuclear extract, and optionally, each separate linear nucleic acid comprises one, two, three, 4, 5, 6, 7, 8, 9, or 10 or more genes or enzyme-encoding sequences, and optionally the linear nucleic acid is present in an extract/product of manufacture at a concentration of about 10 nM (nanomolar), 15 nM, 20 nM, 25 nM, 30 nM, 35 nM, 40 nM, 45 nM or 50 nM or more or between about 1 nM and 100 nM.

In alternative embodiments, products of manufacture (including the mixture of at least two cytoplasmic or nuclear extracts from at least two different cells) and processes as provided herein are fabricated or designed to synthesize in vitro polyketides and non-ribosomally synthesized peptides, two important classes of natural products with a wide range of biological activities are. Both are synthesized in an assembly-line fashion by multidomain multimodular polyketide synthases (PKS) and nonribosomal peptide synthetases (NRPS), and products of manufacture as provided herein comprise nucleic acids encoding multidomain multimodular PKSs or NRPSs, and/or the enzyme polypeptides. These PKS and NRPS enzymes are types of molecular assembly lines that synthesize a precursor molecule by successive incorporation of polyketide extender units for polyketides and amino acids for non-ribosomally synthesized peptides (see Example 1, below). These enzyme classes have a highly modularized organization where each module contains functional domains which catalyze reactions for the biosynthesis of the core molecule. In alternative embodiments, products of manufacture use these modular and assembly-line nature of PKSs and NRPSs for combinatorial biosynthesis to synthesize valuable and novel non-natural analogs. In alternative embodiments, products of manufacture genes, modules and domains are mixed and matched to generate derivatives or new compounds.

### Generating Extracts for TX-TL Expression Systems

In alternative embodiments, the mixture of at least two cytoplasmic or nuclear extracts comprises: at least one extract from a bacterial cell and at least one extract from a eukaryotic cell; at least one extract from a prokaryotic cell and at least one extract from a mammalian cell; at least one extract from a bacterial cell and at least one extract from an insect, a plant, a fungal or a yeast cell; or, extracts from at least two different bacterial cells, two different fungal cells; two different yeast cells, two different insect cells, two different plant cells or two different mammalian cells. In alternative embodiments, the mixture of at least two cytoplasmic or nuclear extracts comprises a mixture of a cytoplasmic and a nuclear extract; a mixture of two different cytoplasmic extracts; or a mixture of at least two different nuclear extracts. In alternative embodiments, at least one of the cytoplasmic or nuclear extracts comprises an extract from or comprises an extract derived from: an Escherichia or a Escherichia coli (E. coli); a Streptomyces or an Actinobacteria; an Ascomycota, Basidiomycota, or a Saccharomycetales; a Penicillium or a Trichocomaceae; a Spodoptera, a Spodoptera frugiperda, a Trichoplusia or a Trichoplusia ni; a Poaceae, a Triticum, or a wheat germ; a rabbit reticulocyte or a HeLa cell. In alternative embodiments, at least one of the cytoplasmic or nuclear extracts comprises an extract from or comprises an extract derived from an E. coli; and, at least one of the cytoplasmic or nuclear extracts comprises an extract from or comprises an extract derived from a Streptomyces.

In alternative embodiments, at least one of the cytoplasmic or nuclear extracts comprises an extract from or comprises an extract derived from: any prokaryotic and eukaryotic organism including, but not limited to, bacteria, including Archaea, eubacteria, and eukaryotes, including yeast, plant, insect, animal, and mammal, including human cells. In alternative embodiments, at least one of the cytoplasmic or nuclear extracts comprises an extract from or comprises an extract derived from: Escherichia coli, Saccharomyces cerevisiae, Saccharomyces kluyveri, Candida boidinii, Clostridium kluyveri, Clostridium acetobutylicum, Clostridium beijerinckii, Clostridium saccharoperbutylacetonicum, Clostridium perfringens, Clostridium difficile, Clostridium botulinum, Clostridium tyrobutyricum, Clostridium tetanomorphum, Clostridium tetani, Clostridium propionicum, Clostridium aminobutyricum, Clostridium subterminale, Clostridium sticklandii, Ralstonia eutropha, Mycobacterium bovis, Mycobacterium tuberculosis, Porphyromonas gingivalis, Arabidopsis thaliana, Thermus thermophilus, Pseudomonas species, including Pseudomonas aeruginosa, Pseudomonas putida, Pseudomonas stutzeri, Pseudomonas fluorescens, Homo sapiens, Oryctolagus cuniculus, Rhodobacter spaeroides, Thermoanaerobacter brockii, Metallosphaera sedula, Leuconostoc mesenteroides, Chloroflexus aurantiacus, Roseiflexus castenholzii, Erythrobacter, Simmondsia chinensis, Acinetobacter species, including Acinetobacter calcoaceticus and Acinetobacter baylyi, Porphyromonas gingivalis, Sulfolobus tokodaii, Sulfolobus solfataricus, Sulfolobus acidocaldarius, Bacillus subtilis, Bacillus cereus, Bacillus megaterium, Bacillus brevis, Bacillus pumilus, Rattus norvegicus, Klebsiella pneumonia, Klebsiella oxytoca, Euglena gracilis, Treponema denticola, Moorella thermoacetica, Thermotoga maritima, Halobacterium salinarum, Geobacillus stearothermophilus, Aeropyrum pernix, Sus scrofa, Caenorhabditis elegans, Corynebacterium glutamicum, Acidaminococcus fermentans, Lactococcus lactis, Lactobacillus plantarum, Streptococcus thermophilus, Enterobacter aerogenes, Candida, Aspergillus terreus, Pedicoccus pentosaceus, Zymomonas mobilus, Acetobacter pasteurians, Kluyveromyces lactis, Eubacterium barkeri, Bacteroides capillosus, Anaerotruncus colihominis, Natranaerobius thermophilusm, Campylobacter jejuni, Haemophilus influenzae, Serratia marcescens, Citrobacter amalonaticus, Myxococcus xanthus, Fusobacterium nuleatum, Penicillium chrysogenum, marine gamma proteobacterium, butyrate-producing bacterium, Nocardia iowensis, Nocardia farcinica, Streptomyces griseus, Schizosaccharomyces pombe, Geobacillus thermoglucosidasius, Salmonella typhimurium, Vibrio cholera, Heliobacter pylori, Nicotiana tabacum, Oryza sativa, Haloferax mediterranei, Agrobacterium tumefaciens, Achromobacter denitrificans, Fusobacterium nucleatum, Streptomyces clavuligenus, Acinetobacter baumanii, Mus musculus, Lachancea kluyveri, Trichomonas vaginalis, Trypanosoma brucei, Pseudomonas stutzeri, Bradyrhizobium japonicum, Mesorhizobium loti, Bos taurus, Nicotiana glutinosa, Vibrio vulnificus, Selenomonas ruminantium, Vibrio parahaemolyticus, Archaeoglobus fulgidus, Haloarcula marismortui, Pyrobaculum aerophilum, Mycobacterium smegmatis MC2 155, Mycobacterium avium subsp. paratuberculosis K-10, Mycobacterium marinum M, Tsukamurella paurometabola DSM 20162, Cyanobium PCC7001, Dictyostelium discoideum AX4.

In alternative embodiments, at least one of the cytoplasmic or nuclear extracts comprises an extract from or comprises an extract derived from: Acinetobacter baumannii Naval-82, Acinetobacter sp. ADP1, Acinetobacter sp. strain M-1, Actinobacillus succinogenes 130Z, Allochromatium vinosum DSM180, Amycolatopsis methanolica, Arabidopsis thaliana, Atopobium parvulum DSM 20469, Azotobacter vinelandii DJ, Bacillus alcalophilus ATCC 27647, Bacillus azotoformans LMG 9581, Bacillus coagulans 36D1, Bacillus megaterium, Bacillus methanolicus MGA3, Bacillus methanolicus PB1, Bacillus methanolicus PB-1, Bacillus selenitireducens MLS10, Bacillus smithii, Bacillus subtilis, Burkholderia cenocepacia, Burkholderia cepacia, Burkholderia multivorans, Burkholderia pyrrocinia, Burkholderia stabilis, Burkholderia thailandensis E264, Burkholderiales bacterium Joshi_001, Butyrate-producing bacterium L2-50, Campylobacter jejuni, Candida albicans, Candida boidinii, Candida methylica, Carboxydothermus hydrogenoformans, Carboxydothermus hydrogenoformans Z-2901, Caulobacter sp. AP07, Chloroflexus aggregans DSM 9485, Chloroflexus aurantiacus J-10-fl, Citrobacter freundii, Citrobacter koseri ATCC BAA-895, Citrobacter youngae, Clostridium, Clostridium acetobutylicum, Clostridium acetobutylicum ATCC 824, Clostridium acidurici, Clostridium aminobutyricum, Clostridium asparagiforme DSM 15981, Clostridium beijerinckii, Clostridium beijerinckii NCIMB 8052, Clostridium bolteae ATCC BAA-613, Clostridium carboxidivorans P7, Clostridium cellulovorans 743B, Clostridium difficile, Clostridium hiranonis DSM 13275, Clostridium hylemonae DSM 15053, Clostridium kluyveri, Clostridium kluyveri DSM 555, Clostridium ljungdahli, Clostridium ljungdahlii DSM 13528, Clostridium methylpentosum DSM 5476, Clostridium pasteurianum, Clostridium pasteurianum DSM 525, Clostridium perfringens, Clostridium perfringens ATCC 13124, Clostridium perfringens str. 13, Clostridium phytofermentans ISDg, Clostridium saccharobutylicum, Clostridium saccharoperbutylacetonicum, Clostridium saccharoperbutylacetonicum NI-4, Clostridium tetani, Corynebacterium glutamicum ATCC 14067, Corynebacterium glutamicum R, Corynebacterium sp. U-96, Corynebacterium variabile, Cupriavidus necator N-1, Cyanobium PCC7001, Desulfatibacillum alkenivorans AK-01, Desulfitobacterium hafniense, Desulfitobacterium metallireducens DSM 15288, Desulfotomaculum reducens MI-1, Desulfovibrio africanus str. Walvis Bay, Desulfovibrio fructosovorans JJ, Desulfovibrio vulgaris str. Hildenborough, Desulfovibrio vulgaris str. ‘Miyazaki F’, Dictyostelium discoideum AX4, Escherichia coli, Escherichia coli K-12, Escherichia coli K-12 MG1655, Eubacterium hallii DSM 3353, Flavobacterium frigoris, Fusobacterium nucleatum subsp. polymorphum ATCC 10953, Geobacillus sp. Y4.JMCI, Geobacillus themodenitrificans NG80-2, Geobacter bemidjiensis Bem, Geobacter sulfurreducens, Geobacter sulfurreducens PCA, Geobacillus stearothermophilus DSM 2334, Haemophilus influenzae, Helicobacter pylori, Homo sapiens, Hydrogenobacter thermophilus, Hydrogenobacter thermophilus TK-6, Hyphomicrobium denitrificans ATCC 51888, Hyphomicrobium zavarzinii, Klebsiella pneumoniae, Klebsiella pneumoniae subsp. pneumoniae MGH 78578, Lactobacillus brevis ATCC 367, Leuconostoc mesenteroides, Lysinibacillus fusiformis, Lysinibacillus sphaericus, Mesorhizobium oti MAFF303099, Metallosphaera sedula, Methanosarcina acetivorans, Methanosarcina acetivorans C2A, Methanosarcina barkeri, Methanosarcina mazei Tuc01, Methylobacter marinus, Methylobacterium extorquens, Methylobacterium extorquens AM1, Methylococcus capsulatas, Methylomonas aminofaciens, Moorella thermoacetica, Mycobacter sp. strain JCJ DSM 3803, Mycobacterium avium subsp. paratuberculosis K-10, Mycobacterium bovis BCG, Mycobacterium gastri, Mycobacterium marinum M, Mycobacterium smegmatis, Mycobacterium smegmatis MC2 155, Mycobacterium tuberculosis, Nitrosopumilus salaria BD31, Nitrososphaera gargensis Ga9.2, Nocardia farcinica IFM 10152, Nocardia iowensis (sp. NRRL 5646), Nostoc sp. PCC 7120, Ogataea angusta, Ogataea parapolymorpha DL-1 (Hansenula polymorpha DL-1), Paenibacillus peoriae KCTC 3763, Paracoccus denitrificans, Penicillium chrysogenum, Photobacterium profundum 3TCK, Phytofermentans ISDg, Pichia pastoris, Picrophilus torridus DSM9790, Porphyromonas gingivalis, Porphyromonas gingivalis W83, Pseudomonas aeruginosa PA01, Pseudomonas denitrificans, Pseudomonas knackmussii, Pseudomonas putida, Pseudomonas sp, Pseudomonas syringae pv. syringae B728a, Pyrobaculum islandicum DSM4184, Pyrococcus abyssi, Pyrococcus furiosus, Pyrococcus horikoshii OT3, Ralstonia eutropha, Ralstonia eutropha H16, Rhodobacter capsulatus, Rhodobacter sphaeroides, Rhodobacter sphaeroides ATCC 17025, Rhodopseudomonas palustris, Rhodopseudomonas palustris CGA009, Rhodopseudomonas palustris DX-1, Rhodospirillum rubrum, Rhodospirillum rubrum ATCC 11170, Ruminococcus obeum ATCC 29174, Saccharomyces cerevisiae, Saccharomyces cerevisiae S288c, Salmonella enterica, Salmonella enterica subsp. enterica serovar Typhimurium str. LT2, Salmonella enterica typhimurium, Salmonella typhimurium, Schizosaccharomyces pombe, Sebaldella termitidis ATCC 33386, Shewanella oneidensis MR-1, Sinorhizobium meliloti 1021, Streptomyces coelicolor, Streptomyces griseus subsp. griseus NBRC 13350, Sulfolobus acidocalarius, Sulfolobus solfataricus P-2, Synechocystis str. PCC 6803, Syntrophobacter fumaroxidans, Thauera aromatica, Thermoanaerobacter sp. X514, Thermococcus kodakaraensis, Thermococcus litoralis, Thermoplasma acidophilum, Thermoproteus neutrophilus, Thermotoga maritima, Thiocapsa roseopersicina, Tolumonas auensis DSM 9187, Trichomonas vaginalis G3, Trypanosoma brucei, Tsukamurella paurometabola DSM 20162, Vibrio cholera, Vibrio harveyi ATCC BAA-1116, Xanthobacter autotrophicus Py2, Yersinia intermedia, or Zea mays.

In alternative embodiments, Actinobacteria or Streptomyces are used as a source of at least one of the extracts because Actinomycetes are important sources of novel bioactive compounds which serve as potential drug candidates for antibiotics development. More than 10,000 bioactive bacterial compounds have been described from the well characterized Actinomycete genus Streptomyces, a significant fraction of the known 18,000 bioactive compounds. Many well-known antibiotics including tetracycline and streptomycin originate from the secondary metabolism of Actinomycetes. Recent sequence analysis studies conducted on multiple Actinomycetes suggests that each bacterium can produce approximately 10-fold more secondary metabolites than has been detected during screening efforts before the corresponding genome sequences were available.

In alternative embodiments, because they are well characterized, Escherichia coli or Saccharomyces cerevisiae are used as a source of at least one of the cytoplasmic or nuclear extracts. In alternative embodiments, Escherichia coli or Saccharomyces cerevisiae are mixed/used with Actinobacteria or Streptomyces extracts; for example, between about 50% and 99.9%, or about 50%, 51%, 52%, 53%, 54%, 55%, 56%, 57%, 58%, 59%, ⁶⁰, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% of the liquid volume of the extract can be from one of the two extracts (from one of the cytoplasmic or nuclear extracts).

In alternative embodiments, Actinomycetes are used as a source of at least one of the extracts, or as a source of nucleic acids to be used in products of manufacture or processes as provided herein, because e.g., they comprise so-called “secondary metabolite gene clusters (SMBGCs) (see discussion, below), and “tailoring” enzymes such as glycosyltransferases, halogenases, methyltransferases and hydroxylases. Thus, Actinomycetes extracts provide biosynthetic precursor synthesis capabilities that support secondary metabolite biosynthesis.

### Supplementing Extracts for TX-TL Expression Systems

In alternative embodiments, products of manufacture, e.g., including at least one of the cytoplasmic or nuclear extracts, have added to them, or further comprise, additional ingredients, compositions or compounds, reagents, ions or element, buffers and/or solutions. In alternative embodiments, processes as provided herein use or fabricate environmental conditions to optimize product of a product, e.g., natural product (NPs) or natural product analogs (NPAs).

For example, in alternative embodiments, products of manufacture, e.g., including at least one of the cytoplasmic or nuclear extracts provided herein are used for the production of an organic molecule, e.g., a violacein, a butadiene or a 1,4-BDO, the extracts or production system are supplemented with a carbon source and other essential nutrients.

In alternative embodiments, processes maintain anaerobic conditions; such conditions can be obtained, for example, by first sparging the medium with nitrogen and then sealing the wells or reaction containers.

If desired, the pH of the production system (e.g., mixed extract as provided herein) can be maintained at a desired pH, in particular neutral pH, such as a pH of around 7 by addition of a buffer, a base, such as NaOH or other bases, or an acid, as needed to maintain the production system at a desirable pH.

The production system (e.g., mixed extract as provided herein) can include, for example, any carbohydrate source. Such sources of sugars or carbohydrate substrates include glucose, xylose, maltose, arabinose, galactose, mannose, fructose, sucrose and starch.

In alternative embodiments, products of manufacture, e.g., including the at least one of the cytoplasmic or nuclear extracts, have added to them, or further comprise one or more enzymes (or the nucleic acids that encode them) of central metabolism pathways, for example, one or more (or all of the) central metabolism enzymes from the tricarboxylic acid cycle (TCA, or Krebs cycle), the glycolysis pathway or the Citric Acid Cycle.

For example, in alternative embodiments, a reductive (reverse) tricarboxylic acid cycle coupled with carbon monoxide dehydrogenase and/or hydrogenase activities for the conversion of CO, CO2 and/or H2 to acetyl-CoA and other products such as acetate are added. Added enzymes (or the nucleic acids that encode them) can include: ATP citrate-lyase, citrate lyase, aconitase, isocitrate dehydrogenase, alpha-ketoglutarate: ferredoxin oxidoreductase, succinyl-CoA synthetase, succinyl-CoA transferase, fumarate reductase, fumarase, malate dehydrogenase, NAD(P)H: ferredoxin oxidoreductase, carbon monoxide dehydrogenase, and/or hydrogenase. For example, the reducing equivalents extracted from CO and/or H2 by carbon monoxide dehydrogenase and hydrogenase are utilized to fix CO2 via the reductive TCA cycle into acetyl-CoA or acetate. Acetate can be converted to acetyl-CoA by enzymes such as acetyl-CoA transferase, acetate kinase/phosphotransacetylase, and acetyl-CoA synthetase. Acetyl-CoA can be converted to the butadiene, glyceraldehyde-3-phosphate, phosphoenolpyruvate, and pyruvate, by pyruvate: ferredoxin oxidoreductase and the enzymes of gluconeogenesis.

In alternative embodiments, enzymes that can be added to supplement the amount of or the production of, for example, acetoacetyl-CoA, 3-hydroxybutyryl-CoA, crotonyl-CoA, crotonaldehyde, crotyl alcohol, 2-betenyl-phosphate, 2-butenyl-4-diphosphate, erythritol-4-phosphate, 4-(cytidine 5′-diphospho)-erythritol, 2-phospho-4-(cytidine 5′-diphospho)-erythritol, erythritol-2,4-cyclodiphosphate, 1-hydroxy-2-butenyl 4-diphosphate, butenyl 4-diphosphate, 2-butenyl 4-diphosphate, 3-oxoglutaryl-CoA, 3-hydroxyglutaryl-CoA, 3-hydroxy-5-oxopentanoate, 3,5-dihydroxy pentanoate, 3-hydroxy-5-phosphonatooxypentanoate, 3-hydroxy-5-[hydroxy (phosphonooxy) phosphoryl]oxy pentanoate, crotonate, erythrose, erythritol, 3,5-dioxopentanoate or 5-hydroxy-3-oxopentanoate.

### Methods for Screening for Enzyme Activity and Isolating Products

In alternative embodiments, any method for screening for a desired enzyme activity, e.g., production of a desired product, e.g., such as a violacein, or a butadiene or 1,4-BDO, can be used. Any method for isolating enzyme products or final products, e.g., natural products, can be used, e.g., as described in: WO2013071226A1 published 16 May 2013 entitled Eukaryotic Organisms and Methods for Increasing the Availability of Cytosolic Acetyl-CoA, and for Producing 1,3-Butanediol; WO2013028519A1 published 28 Feb. 2013 entitled Microorganisms and Methods for Producing 2,4-Pentadienoate, Butadiene, Propylene, 1,3-Butanediol and Related Alcohols.

In alternative embodiments, compositions and methods as provided herein comprise use of any method or apparatus to detect an organic volatile, e.g., butadiene or H2 gas, or a microbial-produced organic volatile (e.g., butadiene gas), by e.g., employing invasive sampling of either extract or headspace followed by subjecting the sample to gas chromatography or liquid chromatography often coupled with mass spectroscopy. In alternative embodiments, any “state-of-the-art” apparatus can be used, e.g., for high throughput” screening, e.g., an Agilent 7697A HEADSPACE SAMPLER™ (Agilent Technologies, Santa Clara CA, USA) having a 111-vial capacity (10 mL, 20 mL, or 22 mL vials) and three 36-vial racks that can be exchanged while the headspace sampler is operating, or equivalent, can be used. In addition to limited sample configurations and numbers, the apparatus when coupled with GC or GC/MS would typically require 10-30 minutes to analyze each sample.

In alternative embodiments, apparatus are designed or configured for High Throughput Screening (HTS) of products, e.g., natural products, e.g., a violacein, a butadiene, produced by products of manufacture or processes as provided herein, by detecting and/or measuring the products, e.g., natural products, either directly or indirectly, e.g., by chemical or enzymatic reaction, e.g., in a soluble form in the cell culture medium, in a gas form in the extract headspace.

In alternative embodiments, methods are automatable and suitable for use with laboratory robotic systems, eliminating or reducing operator involvement, while proving high-throughput screening. In some embodiments the apparatus exploit the volatile nature of products, e.g., volatile natural products, either by direct detection in extract headspace or by trapping the off-gas followed by its detection in the trapped state.

Also provided are methods for screening the chemical, a protein, a small molecule, a natural product, a natural product analog, a secondary metabolite or secondary metabolite analog, or a lipid or a library of one or more of said compounds, produced by a TX-TL system, for an activity of interest. For example, the activity can be for a pharmaceutical, nutraceutical, nutrional or animal veterinary or health function. The activity can be as a modulator of protein activity, transcription, translation or cell function; a toxic metabolite or a protein; a cellular toxin; an inhibitor of transcription or translation.

Also provided are methods screening for: a modulator of protein activity, transcription, or translation or cell function; a toxic metabolite or a protein; a cellular toxin; an inhibitor or of transcription or translation, comprising: (a) providing a TX-TL composition described herein (a product of manufacture), wherein the composition comprises at least one protein-encoding nucleic acid; (b) providing a test compound; (c) combining or mixing the test compound with the product of manufacture under conditions wherein the TX-TL extract initiates or completes transcription and/or translation, or modifies a molecule (optionally a protein, a small molecule, a natural product (NP), natural product analog (NPA) or secondary metabolite or analog thereof or a lipid) and (d) determining or measuring any change in the functioning or products of the extract, or the transcription and/or translation, wherein determining or measuring a change in the protein activity, transcription or translation or cell function identifies the test compound as a modulator of that protein activity, transcription or translation or cell function.

### Identifying and Engineering Metabolic Pathways

In alternative embodiments, provided are methods of identifying and/or modifying an enzyme-encoding natural-product (NP) synthesizing operon; a biosynthetic gene cluster; a plurality of enzyme-encoding nucleic acids for at least two, several or all of the steps in the synthesis of a natural product analog (NPA), or a secondary metabolite analog. In alternative embodiments, provided are engineered or modified enzyme-encoding natural-product (NP) synthesizing operons; biosynthetic gene clusters; and/or enzyme-encoding nucleic acids for at least two, several or all of the steps in the synthesis of a natural product analog (NPA), or a secondary metabolite analog, or libraries thereof, made by these methods. In alternative embodiments, provided libraries of NPs, NPAs, and secondary metabolite analogs made by these methods, and compositions as provided herein. In alternative embodiments, these modifications comprise one or more combinatorial modifications that result in generation of desired NPAs or secondary metabolites, or libraries of NPAs or secondary metabolites.

In alternative embodiments, the one or more combinatorial modifications comprise deletion or inactivation of a module, or one or more individual genes, in a gene cluster for the biosynthesis, or altered biosynthesis, of a natural product (NP) or a secondary metabolite, or an NP analog (NPA) or secondary metabolite analog.

In alternative embodiments, the one or more combinatorial modifications comprise domain engineering to fuse protein (e.g., enzyme) domains, shuffle domains, add an extra domain, exchange of one or more (multiple) domains, or other modifications to alter substrate activity or specificity of an enzyme involved in the biosynthesis or modification of the natural product (NP) or a secondary metabolite, or an NP analog (NPA) or secondary metabolite analog.

In alternative embodiments, the one or more combinatorial modifications comprise modifying, adding or deleting a “tailoring” enzyme that acts after the biosynthesis of a core backbone of the natural product (NP) or secondary metabolite is completed, optionally comprising a methyl transferase, a glycosyl transferase, a halogenase, a hydroxylase, a dehydrogenase. In this embodiment NP analogs (NPAs) or secondary metabolite analogs are generated by the action (e.g., modified action, additional action, or lack of action (as compared to wild type)) of the “tailoring” enzymes.

In alternative embodiments, the one or more combinatorial modifications comprise combining modules from various sources to construct artificial gene synthesis clusters, or modified NP gene synthesis clusters.

In alternative embodiments, enzyme-encoding natural-product (NP) synthesizing operons; biosynthetic gene clusters; enzyme-encoding nucleic acids for at least two, several or all of the steps in the synthesis of a natural product analog (NPA), or a secondary metabolite, for use in products of manufacture or processes as provided herein, are identified by methods comprising e.g., use of: a genomic or biosynthetic search engine, optionally WARP DRIVE BIO™ software, antiSMASH (ANTISMASH™) software, iSNAP™ algorithm (Ibrahim et al., Dereplicating nonribosomal peptides using an informatic search algorithm for natural products (iSNAP) discovery, PNAS 2012 Nov. 20; 109 (47)), CLUSTSCAN™ (Starcevic, et al., (2008) Nucleic Acids Res., 36, 6882-6892), NP searcher (Li et al. (2009) Automated genome mining for natural products. BMC Bioinformatics, 10, 185), SBSPKS™ (Anand, et al. (2010) Nucleic Acids Res., 38, W487-W496), BAGEL3™ (Van Heel, et al., (2013) Nucleic Acids Res., 41, W448-W453), SMURF™ (Khaldi et al., (2010) Fungal Genet. Biol., 47, 736-741), ClusterFinder (CLUSTERFINDER™) or ClusterBlast (CLUSTERBLAST™) algorithms, or a combination there of; or, an Integrated Microbial Genomes (IMG)-ABC system (DOE Joint Genome Institute (JGI)).

In alternative embodiments, products of manufacture as provided herein use (incorporate, or comprise) protein machinery that is responsible for the biosynthesis of secondary metabolites in bacteria; this “machinery” can comprise enzymes encoded by gene clusters or operons. In alternative embodiments, so-called “secondary metabolite gene clusters (SMBGCs) are used; they contain all the genes required for the biosynthesis, regulation and/or export of a product, e.g., a natural product. In vivo genes are encoded (physically located) side-by-side, and they can be used in this “side-by-side” orientation in (e.g., linear or circular) nucleic acids used in products of manufacture and processes as provided herein, or they can be rearranged, or segmented into one or more linear or circular nucleic acids.

For identifying secondary metabolite biosynthesis pathways, bacteria are used as they normally organize the biosynthesis of secondary metabolites in SMBGCs. Fungal sources can be used because some SMBCs have been found in fungal producers. Any software tools for the identification of these gene clusters can be used, e.g., tools for the identification of clusters synthesizing antibiotics and secondary metabolites analysis, e.g., WARP DRIVE BIO™ software, antiSMASH (ANTISMASH™) software, iSNAP algorithm (iSNAP™), CLUSTSCAN software (CLUSTSCAN™), NP searcher, SBSPKS software (SBSPKS™), BAGEL3 software (BAGEL3™), SMURF software (SMURF™), ClusterFinder (CLUSTERFINDER™) or ClusterBlast (CLUSTERBLAST™) algorithms, or a combination thereof, or, an Integrated Microbial Genomes (IMG)-ABC system (DOE Joint Genome Institute (JGI)).

In alternative embodiments, natural product gene clusters for use in TX-TL systems and processes as provided herein are identified from genome sequences of known natural product producers using established genome mining tools, such as antiSMASH, as described in e.g., Weber et al. Nucleic Acids Res. 2015 Jul. 1; 43 (W1): W237-43. These genome mining tools can also be used to identify novel biosynthetic genes (for use in TX-TL systems and processes as provided herein) within metagenomic based DNA sequences.

In alternative embodiments, the identified natural product gene clusters and/or biosynthetic genes are ‘refactored’, e.g., where the native regulatory parts (e.g. promoter, RBS, terminator, codon usage etc.) are replaced e.g., by synthetic, orthogonal regulation with the goal of optimization of enzyme expression in an extract product of manufacture as provided herein and/or in a heterologous host. In alternative embodiments, refactored gene clusters are modified and combined for the biosynthesis of other natural product analogs (combinatorial biosynthesis). In alternative embodiments, refactored gene clusters are added to a TX-TL product of manufacture or process reactions as provided herein, and they can be in the form of linear or circular, e.g., plasmid, DNA.

In alternative embodiments, refactoring strategies comprise changes in a start codon, for example, for Streptomyces it might be advantageous to change the start codon, e.g., to TTG. For Streptomyces it has been shown that genes starting with TTG are better transcribed than genes starting with ATG or GTG (see e.g., Myronovskyi et al., Applied and Environmental Microbiology. 2011; 77: 5370-5383).

In alternative embodiments, refactoring strategies comprise changes in ribosome binding sites (RBSs), and RBSs and their relationship to a promoter, e.g., promoter and RBS activity can be context dependent. For example, the rate of transcription can be decoupled from the contextual effect by using ribozyme-based insulators between the promoter and the RBS to create uniform 5′-UTR ends of mRNA, e.g., as described by Lou, et al., Ribozyme-based insulator parts buffer synthetic circuits from genetic context. Nat Biotechnol. 2012; 30:1137-42.

In alternative embodiment, exemplary processes and protocols for the functional optimization of gene clusters by combinatorial design and assembly comprise methods described herein including next generation sequencing and identification of genes, genes clusters and networks, and gene recombineering (recombination-mediated genetic engineering) e.g. as described by Smanski et al., Nat. Biotechnol. (2014) 32:1241-1249.

In parallel, refactored linear DNA fragments can also be cloned into a suitable expression vector for transformation into a heterologous expression host. In alternative embodiments, provided are TX-TL reactions comprising refactored gene clusters with single organism or mixed crude extracts, including the mixture of at least two cytoplasmic or nuclear extracts from at least two different cells as provided herein.

In alternative embodiments, products of these TX-TL reactions are then subject to a suite of “-omics” based approaches including: metabolomics, transcriptomics and proteomics, towards understanding the resulting metabolome, as well as expression of the natural product gene clusters. In alternative embodiments, natural products produced within the TX-TL reactions as provided herein are identified and characterized using a combination of high-throughput mass spectrometry (MS) detection tools as well as chemical and biological based assays. Following the characterization of the TX-TL produced natural product, the corresponding gene clusters may be cloned into a suitable vector for expression and scale up in a heterologous or native expression host. Production can be scaled up in an in vitro bioreactor.

In alternative embodiments, functional or bioinformatic screening methods are used to discover and identify biocatalysts and gene clusters, e.g., small molecule biosynthetic gene clusters, for use in products of manufacture and processes as described herein. Environmental habitats of interest for the discovery of natural products includes soil and marine environments.

In alternative embodiments, metagenomics, the analysis of DNA from a mixed population of organisms, is used to discover and identify biocatalysts and gene clusters, e.g., small-molecule biosynthetic gene clusters. In alternative embodiments, metagenomics is used initially to involve the cloning of either total or enriched DNA directly from the environment (eDNA) into a host that can be easily cultivated, e.g., as described in Handelsman, J. (2004). Microbiol. Mol. Biol. Rev. 68, 669-685. Next generation sequencing (NGS) technologies also can be used e.g., to allow isolated eDNA to be sequenced and analyzed directly from environmental samples, e.g., as described by Shokralla, et al. (2012) Mol. Ecol. 21,1794-1805.

As described herein the TX-TL compositions and methods can produce analogs of known compounds, for example natural product analogs and secondary metabolic structural analogs. Accordingly the TX-TL compositions can be used in the process described herein that generate product diversity. Methods provided herein include a cell free (in vitro) method for making, synthesizing or altering the structure of a compound, composition, organic molecule small molecule or natural product (NP), natural product analog (NPA) or secondary metabolite or library thereof, comprising using the TX-TL compositions described herein. The methods can produce in the TX-TL extract or mixture at least two or more of the altered compounds to create a library of altered compounds; preferably the library is a natural product analog library, prepared, synthesized or modified by a method comprising use of the product of manufacture or the extract mixture described herein or by using the process or method described herein. Also provided is a library of: natural products (NPs) or natural product analogs (NPAs), or structural analogs of a secondary metabolite, or a combination thereof, prepared, synthesized or modified by a method comprising use of a TX-TL extract or extract mixture (a product of manufacture) described herein or by using the process or method described herein.

In alternative embodiments, practicing the invention comprises use of any conventional technique commonly used in molecular biology, microbiology, and recombinant DNA, which are within the skill of the art. Such techniques are known to those of skill in the art and are described in numerous texts and reference works (See e.g., Sambrook et al., “Molecular Cloning: A Laboratory Manual,” Second Edition, Cold Spring Harbor, 1989; and Ausubel et al., “Current Protocols in Molecular Biology,” 1987). Unless defined otherwise herein, all technical and scientific terms used herein have the same meaning as commonly understood by one of ordinary skill in the art to which this invention pertains. For example, Singleton and Sainsbury, Dictionary of Microbiology and Molecular Biology, 2d Ed., John Wiley and Sons, N Y (1994); and Hale and Marham, The Harper Collins Dictionary of Biology, Harper Perennial, NY (1991) provides those of skill in the art with general dictionaries of many of the terms used in the invention. Although any methods and materials similar or equivalent to those described herein find use in the practice of the present invention, the preferred methods and materials are described herein. Accordingly, the terms defined immediately below are more fully described by reference to the Specification as a whole.

Unless defined otherwise herein, all technical and scientific terms used herein have the same meaning as commonly understood by one of ordinary skill in the art to which this invention pertains. Although any methods and materials similar or equivalent to those described herein find use in the practice of the present invention, the preferred methods and materials are described herein. Accordingly, the terms defined immediately below are more fully described by reference to the Specification as a whole.

As used herein, the singular terms “a,” “an,” and “the” include the plural reference unless the context clearly indicates otherwise. Unless otherwise indicated, nucleic acids are written left to right in 5′ to 3′ orientation; amino acid sequences are written left to right in amino to carboxy orientation, respectively. It is to be understood that this invention is not limited to the particular methodology, protocols, and reagents described, as these may vary, depending upon the context they are used by those of skill in the art.

The following examples, and the figures, are intended to clarify the invention, and to demonstrate and further illustrate certain preferred embodiments and aspects without restricting the subject of the invention to the examples and figures.

### EXAMPLES

### Example 1: Exemplary TX-TL Systems

This Example describes making and using exemplary TX-TL systems as provided herein.
I. Extract hosts, strain engineering approaches and cultivation conditions
   “Base” Organism or Chassis Source for Cytoplasmic or Nuclear Extract: The base organism or chassis can include any organism, e.g., any of the organisms for which robust cell-free expression systems have been established. This includes E. coli (BL21, MG1655(K12)), Saccharomyces cerevisiae (yeast), insect, wheat germ, rabbit and human. In one embodiment, E. coli cytoplasmic or nuclear extract is used as it has been shown to be a powerful expression host for the reconstitution of polyketides.

Strain engineering and or modifications to cultivation conditions can be used to improve the performance of TX-TL for the identification of natural products, as well as the combinatorial biosynthesis of analogs as provided herein.

### Strain Modifications to the “Base” Chassis:

Strain engineering approaches may be taken to improve the performance of the base chassis extract in TX-TL for the production of natural products (NPs), NP analogs (NPAs) and secondary metabolite analogs, as well as for the purpose of combinatorial biosynthesis as provided herein.

In alternative embodiments, genome engineering, e.g., combinatorial modification, approaches are used.

### Modifications to Enhance TX-TL Performance:

Transcriptional modularity: The majority of the TX-TL experiments that we have conducted have been in the BL21 Rosetta strain which lacks the T7 RNA polymerase. Consequently, we have relied on E. coli's native RNA polymerase and corresponding sigma factors to drive transcription. Transcriptional modularity may be desired as large transcripts can be put under the control of a strong bacteriophage promoter such as T7 and other smaller genes within a natural product gene cluster may be put under the control of a sigma 70 based promoter.

A plasmid and promoter system based on the pZ Expression System can be used, as described in Lutz, R. & Bujard, H. Independent and tight regulation of transcriptional units in Escherichia coli via the LacR/O, the TetR/O and AraC/I1-I2 regulatory elements. Nucleic Acids Res. 25, 1203-1210 (1997).

In order to improve transcriptional output as well as overall transcriptional modularity the following modifications can be made to the “base” organism:
1. Overexpression of an RNA polymerase, e.g., a T7 or T3 RNA polymerase (purified polymerase may also be added to the TX-TL reaction)
2. Overexpression of E. coli's sigma factors, namely sigma 70 and sigma 54 or other sigma factors (19, 24, 28, 32 and 38) if corresponding promoters are used. Purified sigma factors may also be added.
3. lacI^{q}: Basal expression control can be achieved by the introduction of a mutated promoter of the lacI gene, called lacIQ, that leads to higher levels of expression (almost 10-fold) of LacI.
4. Expression of rare tRNAs.
5. Expression of gamS to improve transcription from linear DNA.

Protein Production:

In alternative embodiments, BL21 is used as a host for protein production; it harbors a plasmid that encodes for rare tRNAs. Base strains can be transformed with a plasmid that expresses tRNAs for 7 rare codons (AGA, AGG, AUA, CUA, GGA, CCC, and CGG). These tRNA genes can be driven by their native or synthetic promoters.

In alternative embodiments, chaperones native to E. coli including DnaK-DnaJ-GrpE and GroEL-GroES are expressed or overexpressed, this may improve overall protein production in TX-TL.

In alternative embodiments, RNaseE mutants (dominant endonuclease in E. coli) are used: RNaseE mutants in the literature have been shown to increase the half-lives of their substrates [15]. These mutants may be used towards enhancing mRNA stability and consequently protein production.

### Overexpression of Heterologous Proteins:

In alternative embodiments, proteins from gene cluster host or other enzymes are expressed or overexpressed, and they can be used for the identification of natural products (NPs) or for combinatorial biosynthesis of NP analogs.

In alternative embodiments, large (e.g., greater than 100 kDa) proteins (e.g. PKSs) required for natural product synthesis are expressed or overexpressed; they may need to be expressed during growth as to allow for more TX-TL transcriptional and translational resources to be allocated for the expression of the rest of the genes within a cluster.

In alternative embodiments, “tailoring enzymes” are expressed or overexpressed; this can be after the biosynthesis of the core natural product to introduce a variety of chemical modifications. Enzymes such as glycosyl-transferases, halogenases, methyltransferases, and hydroxylases can be added; their addition also can introduce additional chemical moieties, or modified chemical moieties.

In alternative embodiments, heterologous (e.g., heterologous to one or both extracts used in a TX/TL) gene clusters are expressed or overexpressed.

In alternative embodiments, CoA ligases are expressed or overexpressed for the generation of acyl-CoA precursors for natural products.

### Post-Translational Modifications:

In alternative embodiments, glycosyltransferases, or other enzymes that catalyze post-translational modifications that are not native to the base organism or chassis, e.g., E. coli, or if used, additional extract, e.g., an Actinomyces or a Streptomyces, are expressed or overexpressed.

In alternative embodiments, PPTases (e.g., PPTase Sfp from Bacillus subtilis and AcpS from E. coli), corresponding homologues and variants. Polyketide synthases cannot be functional unless their apo-acyl carrier proteins (apo-ACPs) are post-translationally modified by covalent attachment of the 4′-phosphopantetheine group to the highly conserved serine residue, and this reaction is catalyzed by phospho-pantetheinyl transferases (PPTases). PPTases covalently attach the phosphopantetheinyl group derived from coenzyme A (CoA) to acyl carrier proteins or peptidyl carrier proteins as part of the enzymatic assembly lines of fatty acid synthases (FAS), polyketide synthases (PKS), and nonribosomal peptide synthetases (NRPS). In alternative embodiments, PPTase Sfp from Bacillus subtilis and AcpS from E. coli are used, they can transfer small molecules of diverse structures from their CoA conjugates to the carrier proteins.

### Redox and ATP Recycling Systems:

In alternative embodiments, redox mechanisms are expressed or overexpressed; redox is recycled by the proteome that's available in the TX-TL reaction; it also can supplemented, e.g., additional redox reagents can be added.

In alternative embodiments, NAD(P)H regeneration is provided for: NAD(P)H regeneration can overexpress sthA, the soluble trans-hydrogenase from E. coli, to assist with the regeneration of redox as it converts NADPH into NADH and vice versa: NADPH+NAD+<=>NADH+NADP+.

In alternative embodiments, for redox regeneration, can express or overexpress glucose dehydrogenase (EC 1.1.1.47-glucose 1-dehydrogenase [NAD(P)+]) with or without sthA overexpression, produces NADPH from D-glucose and can be a way to generate additional redox from glucose that is added to the TX-TL reaction (D-glucose+NAD(P)+=D-glucono-1,5-lactone+NAD(P)H+H+).

In alternative embodiments, for ATP regeneration: can express or overexpress an ATP regeneration system using e.g., E. coli's ATP synthase and/or Gloeobacter rhodopsin; Gloeobacter rhodopsin has been established and may be overexpressed as an additional ATP regeneration system [16].

### Competing Reactions:

In alternative embodiments, the “base” strain pathways that compete for metabolites, energy and redox that are needed for the synthesis of natural products are predicted in silico using e.g., SIMPHENY™ and can be experimentally validated using a combination of '-omics approaches.

In alternative embodiments, once found, these pathways may be eliminated or modified through, e.g., chromosomal gene modifications, including additions or deletions. In alternative embodiments, protein expression is also modified during in vitro synthesis, or cell growth, by the use of synthetic small RNAs that can effectively target the knockdown of multiple genes, as described e.g., in reference [17].

### Central Metabolism:

In alternative embodiments, in order to maximize energy, redox and metabolites for natural product (NP) or NP analog synthesis, the expression of enzymes within central metabolism may need to be overexpressed, added or deleted, optionally with inclusion of mutants with desired properties.

For example, in one embodiment, a mutant 1pdA is expressed, and/or optionally the endogenous 1pdA is replaced with the mutant 1pdA; the lipoamide dehydrogenase gene (1pdA) encoding the E3 subunits of both the pyruvate dehydrogenase and sucAB complexes, is inhibited by NADH, so to improve flux into the TCA cycle, the endogenous 1pdA can be replaced with a mutant 1pdA that is less inhibited by NADH [20].

In alternative embodiments, transcriptional regulators (ArcA, ArcB, Cra, Crp, Cya, Fnr, and Mlc) are deleted, added or overexpressed: this expression can be used to induce or repress central metabolism and have significant effect on metabolic flux. For example, ArcA, a master transcriptional regulator in bacteria, represses the TCA cycle and can be deleted so expression of many genes becomes constitutive.

b. Modifications for Combinatorial Biosynthesis:

1. Overexpression of Heterologous Proteins:

In alternative embodiments, one or more CoA ligases (e.g., can be multiple) are expressed or overexpressed for the generation of acyl-CoA precursors for natural product (NP) analogs (NPAs).

In alternative embodiments, secondary metabolite pathways from different natural product producing organisms are expressed or overexpressed such that the diversity of compounds (e.g., natural product (NP) analogs (NPAs)) synthesized in a TX-TL is increased.

In alternative embodiments, one or more “tailoring enzyme(s)”, which act after the biosynthesis of the core natural product to introduce a variety of chemical modifications, are expressed or overexpressed.

### Modification of “Base” Chassis Culture Conditions:

In alternative embodiments, because the media and fermentation conditions can significantly affect the performance of crude extracts in a TX-TL reaction, cells are grown aerobically, e.g., in 2×yeast extract and tryptone (YT) growth media, and e.g., harvested mid-log to maximize protein production; or, anaerobic conditions can be used as appropriate.

a. Media Condition Modifications:

In alternative embodiments, minimal media is used, e.g., comprising trace metals, casamino acids.

b. Cultivation Based Modifications:

Co-cultivation: In alternative embodiments, enzymes having new properties that come from higher organisms which may not be expressed or are inactive in bacterial hosts are expressed, e.g., in a biosynthetic gene cluster, for use in exemplary TX-TLs as provided herein; enzymes not having properties in a “starting” biosynthetic gene cluster can be added. In alternative embodiments, to address this, E. coli is co-cultured with a Saccharomyces cerevisiae or a yeast e, or extracts from E. coli and Saccharomyces cerevisiae are mixed, or enzymes from Saccharomyces cerevisiae are added to a biosynthetic gene cluster from a bacteria, e.g., an E. coli. E. coli or Saccharomyces may also be co-cultivated with a Streptomyces or any gene cluster containing organism. Alternatively, E. coli and/or Saccharomyces extracts are mixed with an extract from a Streptomyces or from any gene cluster containing organism.

### Crude Extraction of “Base” Chassis:

An exemplary protocol for E. coli can be found in reference [3], below, or Sun, et al. Protocols for Implementing an Escherichia coli Based TX-TL Cell-Free Expression System for Synthetic Biology. J. Vis. Exp. (79), e50762, (2013). An exemplary protocol for yeast based cell-free protein synthesis extract preparation can be found in reference [18].

a. Fractionation of Crude Extracts:

An exemplary E. coli based TX-TL protocol is based on the preparation of an E. coli S30 extract [19]. It may be desired to produce additional fractions including an S100 (high-speed supernatant fraction), 70S ribosomes and 30S and 50S ribosomes. The S100 fraction may have soluble proteins of interest. These fractions can be mixed towards optimizing TX-TL productivities.

Many antibiotics have been shown to be inhibitors of translation. Aminoglycoside antibiotics have an affinity for the 30S ribosome subunit. Streptomycin, one of the most commonly used aminoglycosides, interferes with the creation of the 30S initiation complex; to address this: in alternative embodiments, ribosomal fractions from heterologous organism are added or mixed in (e.g., to the extract of the TX/TL) to reduce the possibility of, or reduce, translational inhibition by a desired natural product the may be an inhibitor of translation, e.g., have an affinity for the 30S ribosome subunit.

b. “-'Omics-Based” Characterization and Optimization of Crude Extracts:

To optimize a TX/TL system, crude or initial cytoplasmic extracts can be subjected to proteomics analysis for the determination of the proteome available for the TX-TL reaction, metabolomics can be used to validate that intracellular metabolites are no longer present and genomics/transcriptomics to validate that endogenous nucleic acids have been successfully digested by exonucleases.

### Gene Cluster Containing Organisms:

In alternative embodiments, natural product (NP) gene clusters are used e.g., in products of manufacture and processes as provided herein; these can be found e.g., in bacteria (e.g. Streptomyces), fungus (e.g. Aspergillus spp.) and plants (e.g. poppy, P. somniferum). For example, an Actinomycetes can be used as a host, or as the source of a cytoplasmic extract, for a secondary metabolite gene cluster, or for the heterologous expression of secondary metabolite gene clusters, as provided herein, as they have available precursors that support NP, NP analog (NPA) and secondary metabolite biosynthesis, and express enzymes that are needed for the modification of natural products, e.g., for NPA syntheses as provided herein.

In alternative embodiments, fungal organisms including Aspergillus spp. which can encode an impressive number of natural product clusters can be used, e.g., as heterologous expression hosts or as the source of a cytoplasmic extract. Organisms containing natural product biosynthetic gene clusters used to practice the products of manufacture or processes as provided herein, e.g., to provide a source of extract, can include any of the following organisms (also, any of these organisms can be used as a source of extract):
Streptomyces (bacteria): S. albus J1074; S. ambofaciens; S. ambo BES2074; S. avermitilis, S. ave SUKA17, S. coelicolor M1154, S. fradiae (tyl), S. roseosporus, S. toyocaensis, S. venezuelae, S. cinnamonensis or equivalents.
Bacteria derived from or bacterial isolates from environmental samples: Aspergillus (Fungus) such as A. niger, A. oryzae, A. nidulans or equivalents.
Plants: P. somniferum, S. lycopersicon, M esculenta, L. japonicas, A. thaliana, Zea mays, Avena spp or equivalents.

In alternative embodiments, ‘cryptic’ gene clusters are induced in an extract source strain. Strain engineering and or modifications to cultivation conditions can be used to improve the performance of TX-TL for the identification of natural products and for the combinatorial biosynthesis of analogs in a gene cluster containing host. Both approaches (strain engineering and or modifications to cultivation conditions) can be used for the induction of ‘cryptic’ gene clusters, e.g., in various Streptomyces hosts or sources of extract; this achieves an additional layer of structural complexity to the synthesized compounds, e.g., NP or NPA, when applied together with combinatorial biosynthesis methods.

### Strain Modifications to the Gene Cluster Containing Organisms:

Strain engineering approaches may be taken to improve the performance of the gene cluster containing organism extract in TX-TL for the production of natural products as well as for the purpose of creating chemical diversity for the combinatorial biosynthesis of analogs.

### Genome Engineering Tools, e.g., for Actinomycetes or Streptomyces Include: CRISPR/Cas System:

In alternative embodiments, an engineered CRISPR/Cas system is used for rapid multiplex genome editing of genes, operons, metabolite gene clusters to practice the products of manufacture and processes as provided herein, e.g., for multiplex genome editing of Streptomyces strains; the editing can comprise activation of genes (e.g., to enhance the expression of a metabolite gene cluster) or targeted chromosomal deletions, e.g., in Streptomyces species, targeted chromosomal deletions of various sizes (ranging from 20 bp to 30 kb); CRISPR/Cas has an efficiency ranging from 70-100% [28]. In alternative embodiments, CRISPR/Cas systems are used for deletions and the activation of genes; and also can be used to either delete or enhance the expression of metabolite gene clusters.

In alternative embodiments, traditional site-specific recombination strategies are used to practice the products of manufacture and processes as provided herein: for example, Two site-specific strategies such as Cre/loxP and Dre/rox (leave scars on the genome) can be used; or, alternatively, a scar-less method can be used where the Dre and Cre recombinases are used in the construction of unmarked multiple mutations, marker-free expression of target genes, large-scale deletions, and/or the chromosomal integration of biosynthetic gene clusters in, e.g., different genera of Actinomycetes (developed as described in [29]).

### Transcriptional Modularity and Control:

In alternative embodiments, native and synthetic promoters are used, e.g., are engineered into genes, e.g., operons or metabolite gene clusters. For example, in a pure Actinomycetes or Streptomyces gene cluster containing cell extract it may be advantageous to utilize native and synthetic promoters for optimal transcriptional performance. If the extract is composed of both an 15 E. coli and a Streptomyces extract, a combination of promoters may be selected, taking advantage of the available transcriptional machinery from E. coli and Streptomyces as well as a bacteriophage orthogonal RNA polymerase. Listed below are an exemplary, base set of promoters that are active in both E. coli and a Streptomyces hosts or E. coli and a Streptomyces-derived extracts which can be used as heterologous expression hosts for the refactored natural product gene clusters.

In alternative embodiments, in order to improve transcriptional output as well as overall transcriptional modularity, the following modifications or additions can be made to the natural product producers or extracts provided or used herein:
Overexpression or addition of an RNA polymerase, e.g., a T7 or T3 RNA polymerase (purified polymerase may also be added to the TX-TL reaction);
Overexpression or addition of gene cluster containing organism, e.g., Streptomyces, sigma factors, e.g., sigma 70 and sigma 54, or other sigma factors (HrdB, 19, 24, 28, 32 and 38) if corresponding promoters are used. Purified sigma factors may also be added;
Exemplary Promoters for Actinomycetes or Streptomyces include: strong promoters, for example, an ermEp* (a heterologous promoter from Saccharopolyspora erythraea and is active in E. coli), a SF14p or a kasOp* (active also in E. coli) [21].
Exemplary Promoters used to practice the invention may be derived from a promoter library, e.g., for Actinomycetes or Streptomyces; libraries of promoters for expression of heterologous and endogenous pathways can be used; for example, a synthetic promoter library can be constructed as for the modulation of actinorhodin production in Streptomyces coelicolor A3(2) as described in reference [22]. In reference [23], the construction of a library of synthetic promoters revealed some specific features of strong Streptomyces promoters. In total 38 promoters of varying strength were characterized.
2. Strain Engineering: Alternative approaches may be taken for the engineering of a host suitable for natural product identification and for the combinatorial biosynthesis of product analogs, and for preparation of a strain as a source of the mixture of at least two cytoplasmic or nuclear extracts from at least two different cells, e.g., for the engineering of an Actinomycetes or Streptomyce.

### Strain Engineering for Natural Product (NP) Identification:

In alternative embodiments, genome-minimized heterologous expression hosts are used, e.g., for the removal of secondary metabolite biosynthetic gene clusters (SMBGCs), e.g., as a genome-minimized Actinomycetes. It may be critical for the identification of novel gene clusters in TX-TL to remove one or more secondary metabolite biosynthetic gene clusters. For example, four endogenous gene clusters for actinorhodin, undecylprodigiosin, coelimycin, and calcium dependent antibiotic (CDA) can be deleted from Streptomyces coelicolor. Point mutations can be introduced into rpoB and rpsL to pleiotropically increase or decrease the level of secondary metabolite production [24]. Different sets of secondary metabolite gene clusters (SMBGCs) can be deleted, e.g., from S. coelicolor, to create a series of genome-reduced hosts, including one strain with all clusters deleted, as described in [25].

In alternative embodiments, systems based approaches (transcriptomics, metabolomics, C13-metabolic flux analysis and proteomics) are used to identify secondary metabolite pathways, e.g., secondary metabolite biosynthetic gene clusters (SMBGCs), for targeted deletions (these modified pathways can then be used in processes as provided herein), as well as for overexpression.

### Deletion or Disruption of Competing Secondary Metabolite Pathways:

Genome-minimized strains, e.g., as described above, can be used; they may be advantageous for the identification of natural products from biosynthetic gene clusters, as they can have all or the majority of SMBGC gene clusters removed, thereby providing a background for which the target compound of interest can be more easily detected.

In alternative embodiments, small RNAs targeting secondary metabolite biosynthesis are used, expressed or added, e.g., to host cells from which extracts are derived; small RNAs can be expressed during the growth of the organism, e.g., expressed prior to the preparation of a crude cytoplasmic extract (e.g., a base extract to be used as described herein) to e.g., achieve a similar expression background as achieved with the genome-minimized strains. Small RNAs as well as CRISPR/Cas technologies may also be used for the combinatorial knockdown of secondary metabolite pathways to provide both a clean secondary metabolite free background for as well as a host suitable for the implementation of combinatorial biosynthesis strategies in TX-TL.

### Strain Engineering for Combinatorial Synthesis Approaches.

Several methods can be used for the induction of cryptic or silent gene clusters. The expression of these gene clusters may create additional chemical diversity that can be explored in the TX-TL reaction on its own or in combination with DNA from refactored gene clusters. The list of possible genetic approaches are listed below and also can be used the identification of natural products.

In alternative embodiments, ribosome and RNA polymerase engineering is used, for example: rpoB and rpsL mutants (components of ribosomal subunits) can be made and/or added to enhance secondary metabolite production, as described e.g., in [24]; mutations to the RNA polymerase machinery can be made to increase promoter binding affinity; deletion or overexpression of pathway or global regulators (activators and repressors); expression of mutant transcriptional regulators; and/or expression or overexpression of ribosome recycling factor (RRF) (rpsL mutants have been shown to induce the expression of RRF and consequently secondary metabolite biosynthesis).

In alternative embodiments, secondary metabolite biosynthetic pathway gene clusters are duplicated, or increased multiple times.

In alternative embodiments, combinations of antibiotic resistance mutations are made, e.g., as described in reference [26], which showed that combinations of antibiotic resistance mutations let to increase in the production of the polyketide actinorhodin (Act).

In alternative embodiments, “epigenetic mining” is used e.g., within higher organisms such as fungi, where epigenetic modifications such as phosphorylation, acetylation, methylation, ubiquitination, ADP-ribosylation, and glycosylation are known to regulate gene expression. Modulating epigenetic control also can be used to enhance or activate production of natural products in eukaryotes, e.g., fungal organisms as in reference [27], and may be desired if fungal hosts are used.

In alternative embodiments, self-resistance is engineered, e.g., self-resistance mechanisms are added or created; these are often encoded in secondary metabolic gene clusters and can influence the yields of secondary metabolites. Self-resistance can be engineered through the upregulation or overexpression of resistance genes such as drrABC, avtAB and actAB, as described in reference [32].

### Protein Production:

In alternative embodiments, rare tRNAs or expressed or overexpressed, e.g., Streptomyces strains can be transformed with a plasmid that expresses tRNAs for 7 rare codons (AGA, AGG, AUA, CUA, GGA, CCC, and CGG). These tRNA genes can be driven by their native or synthetic promoters.

In alternative embodiments, one or more chaperones are expressed or overexpressed, e.g., chaperones native to Streptomyces including DnaK-DnaJ-GrpE and GroEL-GroES may improve overall protein production in TX-TL.

In alternative embodiments, RNaseE is inactivated e.g., by mutation, a dominant endonuclease in bacteria, are generated; RNaseE mutants in the literature have been shown to increase the half-lives of their substrates [15]. These mutants may be used towards enhancing mRNA stability and consequently protein production.

### Overexpression of Native or Heterologous Proteins:

In alternative embodiments, native or heterologous proteins are expressed or overexpressed to identify natural products or for the combinatorial biosynthesis of analogs; for example:

In alternative embodiments, large (for example, greater than 100 kDa) proteins (e.g. polyketide synthases (PKS)) required for natural product synthesis are expressed or overexpressed; these may be needed to be expressed during growth as to allow for more TX-TL transcriptional and translational resources to be allocated for the expression of the rest of the genes within a cluster;

In alternative embodiments, “tailoring enzymes” are expressed or overexpressed; their action after the biosynthesis of the core natural product can introduce a variety of chemical modifications. Enzymes such as glycosyltransferases, halogenases, methyltransferases, and hydroxylases and introduce additional chemical moieties.

In alternative embodiments, CoA ligases are expressed or overexpressed for the generation of acyl-CoA precursors for natural products.

In alternative embodiments, Streptomyces antibiotic regulatory protein (SARP) is expressed or overexpressed for e.g., positive regulation of antibiotic production. Many gene clusters in Streptomyces encode a SARP which has been shown to be a positive regulator of antibiotic production. The overexpression of SARP from a particular Streptomyces host of interest may be useful for the identification of natural products and for combinatorial biosynthesis.

In alternative embodiments, native or heterologous gene clusters are expressed or overexpressed.

In alternative embodiments, MbtH-like proteins are expressed or overexpressed; MbtH-like proteins participate in tight binding to nonribosomal peptide synthetases (NRPS) proteins containing adenylation (A) domains where they stimulate adenylation reactions. Expression of MbtH-like proteins may be important for a number of applications, including optimal production of native and genetically engineered secondary metabolites produced by mechanisms that employ NRPS enzymes.

### Post-Translational Modifications:

In alternative embodiments, phosphopantetheinyl transferases (PPTases), corresponding homologues and variants are expressed or overexpressed. Polyketide synthases cannot be functional unless their apo-acyl carrier proteins (apo-ACPs) are post-translationally modified by covalent attachment of the 4′-phosphopantetheine group to the highly conserved serine residue, and this reaction is catalyzed by phosphopantetheinyl transferases (PPTases). PPTases covalently attach the phosphopantetheinyl group derived from coenzyme A (CoA) to acyl carrier proteins or peptidyl carrier proteins as part of the enzymatic assembly lines of fatty acid synthases (FAS), polyketide synthases (PKS), and nonribosomal peptide synthetases (NRPS). PPTase Sfp from Bacillus subtilis and AcpS from E. coli also transfer small molecules of diverse structures from their CoA conjugates to the carrier proteins.

### Redox and ATP Recycling Systems:

In alternative embodiments, redox mechanisms are expressed or overexpressed; redox is recycled by the proteome that's available in the TX-TL reaction; it also can supplemented, e.g., additional redox reagents can be added.

In alternative embodiments, NAD(P)H regeneration is provided for: NAD(P)H regeneration can overexpress sthA, the soluble trans-hydrogenase from E. coli, to assist with the regeneration of redox as it converts NADPH into NADH and vice versa: NADPH+NAD+<=>NADH+NADP+.

In alternative embodiments, for redox regeneration, can express or overexpress glucose dehydrogenase (EC 1.1.1.47-glucose 1-dehydrogenase [NAD(P)+]) with or without sthA overexpression, produces NADPH from D-glucose and can be a way to generate additional redox from glucose that is added to the TX-TL reaction (D-glucose+NAD(P)+=D-glucono-1,5-lactone+NAD(P)H+H+).

In alternative embodiments, for ATP regeneration: can express or overexpress an ATP regeneration system using e.g., E. coli's ATP synthase and/or Gloeobacter rhodopsin; Gloeobacter rhodopsin has been established and may be overexpressed as an additional ATP regeneration system [16].

### Competing Reactions:

In alternative embodiments, the “base” strain pathways that compete for metabolites, energy and redox that are needed for the synthesis of natural products are predicted in silico using e.g., SIMPHENY™ and can be experimentally validated using a combination of '-omics approaches.

In alternative embodiments, once found, these pathways may be eliminated or modified through, e.g., chromosomal gene modifications, including additions or deletions. In alternative embodiments, protein expression is also modified during in vitro synthesis, or cell growth, by the use of synthetic small RNAs that can effectively target the knockdown of multiple genes [17]. Deletion of secondary metabolite biosynthesis may be critical for the identification of natural products as well as eliminating or reducing the competition for TX-TL resources and metabolites.

### Central Metabolism:

In alternative embodiments, in order to maximize energy, redox and metabolites for natural product (NP) or NP analog synthesis, the expression of enzymes within central metabolism may need to be overexpressed, added or deleted, optionally with inclusion of mutants with desired properties.

For example, in one embodiment, a mutant 1pdA is expressed, and/or optionally the endogenous 1pdA is replaced with the mutant 1pdA; the lipoamide dehydrogenase gene (1pdA) encoding the E3 subunits of both the pyruvate dehydrogenase and sucAB complexes, is inhibited by NADH, so to improve flux into the TCA cycle, the endogenous 1pdA can be replaced with a mutant 1pdA that is less inhibited by NADH [20].

In alternative embodiments, transcriptional regulators (ArcA, ArcB, Cra, Crp, Cya, Fnr, and Mlc) are deleted, added or overexpressed: this expression can be used to induce or repress central metabolism and have significant effect on metabolic flux. For example, ArcA, a master transcriptional regulator in bacteria, represses the TCA cycle and can be deleted so expression of many genes becomes constitutive.

### Modification of Gene Cluster Containing Organism's Culture Conditions:

In alternative embodiments, media and fermentation conditions are modified; they can significantly affect the performance of crude extracts in a TX-TL reaction. Cells can be grown aerobically in 2× yeast extract and tryptone (YT) growth media and harvested mid-log to maximize protein production. These media conditions may be suitable as well for the cultivation of Streptomyces hosts.

In alternative embodiments, exemplary media and cultivation conditions listed below are used for the purpose of identifying natural product gene clusters in TX-TL as well as for the combinatorial biosynthesis of new analogs.

### Exemplary Media and Fermentation Modifications:

Media composition, aeration, culture vessel, addition of enzyme inhibitors; Minimal media, optionally with trace metals, casamino acids; rich media (TB, LB, YT); Modulation of phosphate levels, ammonium, calcium and pH; addition of trace metals, vanadium, Mg2+, Fe; distilled or tap water; addition of rare earth metals (the addition of rare earth metals has been shown to induce secondary metabolite biosynthesis); nutrient depletion (nutrient depletion is coupled with sporulation which could induce secondary metabolite biosynthesis pathways); use of iron-chelators; environmental cues, optionally stress, nutrients, ambient pH, temperature, iron availability, sporulation, oxygen, water and light; the addition of hormones and or antibiotics may induce the production of secondary metabolites; the addition of Mg2+ has been shown to influence the production of secondary metabolites; the addition of inhibitors, such as F-actin inhibitor jasplakinolide, see e.g., reference [30]; heat (42° C.) (thermal stress) or ethanol based stressors; addition of N-acetyl glucosamine (can stimulate antibiotic production); and/or, addition of synthetic compounds, natural product analogs or natural products, small-molecule probes (including e.g., a collection of synthetic small molecules that alter the secondary metabolism of S. coelicor as identified in reference [32], it may be advantageous to add these small-molecule effectors as their addition negates the need for genetic strategies.

### Exemplary Cultivation Based Modifications:

Stimulating secondary metabolite production through changing culture conditions: OSMAC approach (one strain, many compounds): Can use this approach, which examines the ability of single strains to produce different compounds when growing under different conditions, e.g., as described in reference [31], which identified 20 metabolites from a single strain by changing the growth conditions; this approach can be combined with the media recommendations listed above. Co-cultivation: Co-culture can be used to induce the production of cryptic metabolites. In order to achieve a laboratory situation that more closely resembles environmental conditions, co-culture of two or three species can be used; the aim of these co-culturing methods is to mimic environmental conditions that will facilitate the discovery of new secondary metabolites.

The list below includes exemplary Streptomyces co-cultures involved in antibiotic production, that can be used, e.g., also as described in reference [33]: Streptomyces coelicolor-five Actinomycetes; Streptomyces coelicolor-Bacillus subtilis; Streptomyces spp.-Tsukamurella pulmonis; Streptomyces coelicolor-Myxococcus xanthus; Streptomyces clavuligerus-Staphylococcus aureus; Streptomyces cinnabarinus-Alteromonas sp.; Streptomyces sp.-Proteobacteria; Streptomyces coelicolor-Corallococcus coralloides; Streptomyces fradiae 007-Penicillium sp.WC-29-5; Streptomyces lividans-Bacillus subtilis; Streptomyces lividans-Verticillium dahliae; Streptomyces have also been successfully co-cultured with a variety of fungal species including: Streptomyces bullii-Aspergillus fumigatus; Streptomyces rapamycinicus-Aspergillus fumigatus; Streptomyces peucetius-Aspergillus fumigatus; Streptomyces coelicolor-Aspergillus niger.

### Selections for Enhanced Secondary Metabolite Production:

In alternative embodiments, antibiotic selections are carried out for mutations that enhance transcription and translation of secondary metabolite genes in stationary phase.

1.2.3 Crude Extraction of Gene Cluster Containing Organisms:

An exemplary protocol for E. coli can be found in reference [3]. Reference for yeast based cell-free protein synthesis extract preparation can be found in reference [18].

### Exemplary Fractionation Methods for Using or Making Crude, or Source, Extracts:

An exemplary E. coli based TX-TL protocol is based on the preparation of an E. coli S30 extract [19]. S30 extracts have been produced by similar methods for several Streptomyces species. In alternative embodiments, additional fractions including an S100 (high-speed supernatant fraction), 70S ribosomes and 30S and 50S ribosomes are added or produced. The S100 fraction may have soluble proteins of interest. These fractions can be mixed towards optimizing TX-TL productivities. Many antibiotics have been shown to be inhibitors of translation. Aminoglycoside antibiotics have an affinity for the 30S ribosome subunit. Streptomycin, one of the most commonly used aminoglycosides, interferes with the creation of the 30S initiation complex. In alternative embodiments, ribosomal fractions from heterologous organism are mixed to reduce the possibility of translational inhibition by the desired natural product.

### Genome Mining for Natural Product Gene Clusters

### Exemplary Methods and Compositions for Refactoring of Gene Clusters:

Codon optimization also to enhance protein production; List of promoters: lac/lac UV5, tac/trc, PBAD, T7/T71ac, pTET; Refactoring for combinatorial biosynthesis; Ribo J, insulators; Rolling circle amplification to create more DNA; Combinatorial biosynthesis; High GC polymerases, or a combination thereof.

### Exemplary In Vitro Transcription-Translations (TX-TL) Reactions

### Exemplary Methods and Compositions for Enhancing Performance TX-TL:

Length challenges: use modules approximately 4 to 6 kb in length, or more, and need to optimize; add degradation tags to smaller transcripts; resources can be shuttled to larger proteins or tune expression; Amicon filter+partial pathway; gamS mutants to improve protection may or may not be needed; Measurement of transcriptional output and the systems translational with RNA aptamer (and using this platform to screen for TX-TL improvements); Concentrate DNA: DNA lyophilization for TX-TL high-throughput. (larger genes require more DNA); DOE experiments, RiboJ; Amicon filter for larger proteins; Cofactors (SAM, NAD(H), NADP(H)); Metas (Zn, Mg2+); Tailor tRNA mixes; CoA substrates; Dsb; Metals, e.g., Fe and trace metals; Add non-proteinogenic amino acids; Crude extracts, e.g., in many combinations; HT-prep of extracts from hundreds/thousands of matrixed strains (co-culture) and cultivation; approaches towards gaining a new hierarchy of natural products; Multi-pot reactions: also can be used to enhance TX-TL performance; Encapsulation of TX-TL: phospholipid; Protease inhibitors, a cocktail of protease inhibitors may be added to the TX-TL reaction towards reducing proteolytic cleavage of produced proteins; Improving performance with membrane vesicles (can go up to 100 h) and microfluidics. (time and productivity extension), liposomes; Oxygen limitation can be used; pH limitation, try calcium carbonate.

An exemplary TX-TL extract comprises the ingredients, and optionally with the amounts, as set forth in the following Table 1:

| Ingredients | Concentration | |
|---|---|---|
| E. Coli Extract | 33% v/v (about10 mg/ml of protein) | |
| Amino Acid | 1.5 mM (Leucine, 1.25 mM) | |
| HEPES | 50 | mM |
| ATP | 1.5 | mM |
| GTP | 1.5 | mM |
| CTP & UTP | 0.9 | mM |
| tRNA | 0.2 | mg/ml |
| CoA | 0.26 | mM |
| NAD+ | 0.33 | mM |
| CAMP | 0.75 | mM |
| folinic acid | 0.068 | mM |
| spermidine | 1 | mM |
| pEG-8000 | 2% | |
| Mg-glutamate | 4-10 | mM |
| K-glutamate | 8-160 | mM |
| DTT | 5 | mM |
| NADPH | 1 | mM |
| maltodextrin | 35 | mM |
| IPTG | 0.5 | mM |
| pyruvate | 30 | mM |
| NADH | 1 | mM |

### Example 2: Combinatorial Biosynthesis to Generate Novel Natural Products

Provided herein are processes for combinatorial biosynthesis to generate products, e.g., novel natural products, and exemplary processes are described in this Example:

Exemplary Methods for Preparing Cell Extracts of a Streptomyces Strain with the Desired Gene Cluster for Synthesizing Natural Products:

Modification of precursors, for example, making fluoromalonyl-CoA instead of malonyl-CoA that is then used as a precursor by the PKS or the nonribosomal peptide synthetases (NRPS) module, or adding precursors to the extract, e.g. fluoromalonyl-CoA, to substitute in desired reactions instead of native precursors; Deletion or inactivation of modules or domains; Domain engineering to fuse domains, modify them to alter substrate specificity, shuffling of modules, adding a repeat unit; modification of tailoring enzymes that act after the biosynthesis of the core backbone is finished; enzymes include but are not limited to methyl transferases, glycosyl transferases, halogenases and hydroxylases; these can either be inactivated or can be modified to introduce new functional groups at different positions, leading to different compounds; Construction of artificial PKS-NRPS clusters by combination of modules from various sources.

### Combinatorial Biosynthesis of Assembly Line Polyketide Synthases (PKS).

This exemplary protocol makes two basis assumptions that have been validated, as described in: Fong and Khosla, Current opinion in chemical biology, 2012, 16 (117-123).

Individual Enzymes Along the Assembly Line have Relaxed Substrate Specificity.

In alternative embodiments, structurally altered biosynthetic intermediate are used. Substrate specificity of some PKS modules have been quantified and shown to accept many substrate analogs within a Kcat/Km range of 10-100 fold. Therefore, if the reaction in consideration is not rate limiting, a structurally altered biosynthetic intermediate is used.

In alternative embodiments, precursor directed biosynthesis is used to convert unnatural primer units or diketides of a number of natural PKSs into the corresponding polyketide analogs.

In alternative embodiments, module duplication is used; successive modules of certain PKSs show exceptionally high conservations in KS and ACP domain sequences, suggesting that module duplication may have been sufficient for the evolution of long, variably functionalized polyketide backbones.

In alternative embodiments, mechanisms that promote channeling of biosynthetic intermediates from one enzyme to the other are sufficiently conserved to permit the engineering of chimeric assembly lines.

In alternative embodiments, available structural modules for DEBS in other systems such as tryptophan synthase, the intermediates are channeled for very short distances on the order of 1-nM suggest that the growing polyketide chain is channeled across extraordinary lengths (50-100 nM) before the product is released. PKSs rely on selective and dynamic protein-protein interactions.

Description of a PKS modular composition: (McDaniel et al., PNAS, Mar. 2, 1999 vol. 96 no. 5 1846-1851)

In alternative embodiment, provided are products of manufacture and processes using polyketide synthase (PKS) modules, which comprise three domains—a ketosynthase, an acyltransferase (AT), and an acyl carrier protein (ACP)—that catalyze a 2-carbon extension of the growing polyketide chain. The choice of extender unit used by each module-acetate, propionate, or other small organic acids in the form of CoA thioesters—is determined by the specificity of the AT domain. With each 2-carbon chain extension, the final state of the β-carbonyl is embedded as a ketone, hydroxyl, methenyl, or methylene group by the presence or absence of one, two, or three additional catalytic domains in the module—a ketoreductase (KR), dehydratase (DH), and enoyl reductase (ER). In effect, the composition of catalytic domains within a module provides a “code” for the structure of each 2-carbon unit, and the order of modules codes for the sequence of the 2-carbon units, together creating a template colinear with the polyketide product. The remarkable structural diversity of polyketides is governed by the combinatorial possibilities of catalytic domains within each module, the sequence and number of modules, and the post-polyketide synthesis cyclization and “tailoring” enzymes that accompany the PKS genes. The direct correspondence between the catalytic domains of modules in a PKS and the structure of the resulting biosynthetic product is the basis for the exemplary process for modifying polyketide structure by modifying the domains of the modular PKS.

### Example 3: Domain Substitution: Erythromycin Library

Provided herein are processes for combinatorial biosynthesis, including domain substitution, to generate products, e.g., novel natural products, and exemplary processes are described in this Example:

A combinatorial library of polyketides has been constructed by using 6-deoxyerythronolide B synthase (DEBS), the PKS that produces the macrolide ring of erythromycin. This was accomplished by substituting the ATs and β-carbon processing domains of DEBS with counterparts from the rapamycin PKS (RAPS) that encode alternative substrate specificities and β-carbon reduction/dehydration activities. Engineered DEBS containing single, double, and triple catalytic domain substitutions catalyzed production of erythromycin macrolactones with corresponding single, double, and triple modifications. The ability to simultaneously manipulate multiple catalytic centers of the PKS demonstrates the robustness of the engineering process and the potential for creating libraries of novel polyketides that are impractical to prepare in the chemistry laboratory; Protocols are described in e.g., McDaniel et al., PNAS, Mar. 2, 1999 vol. 96 no. 5 1846-1851.

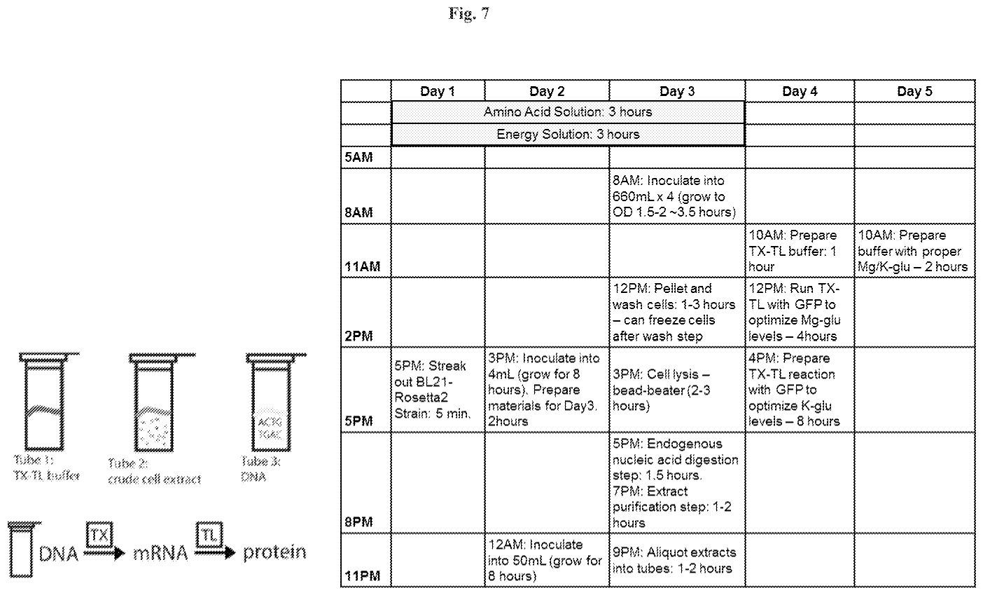

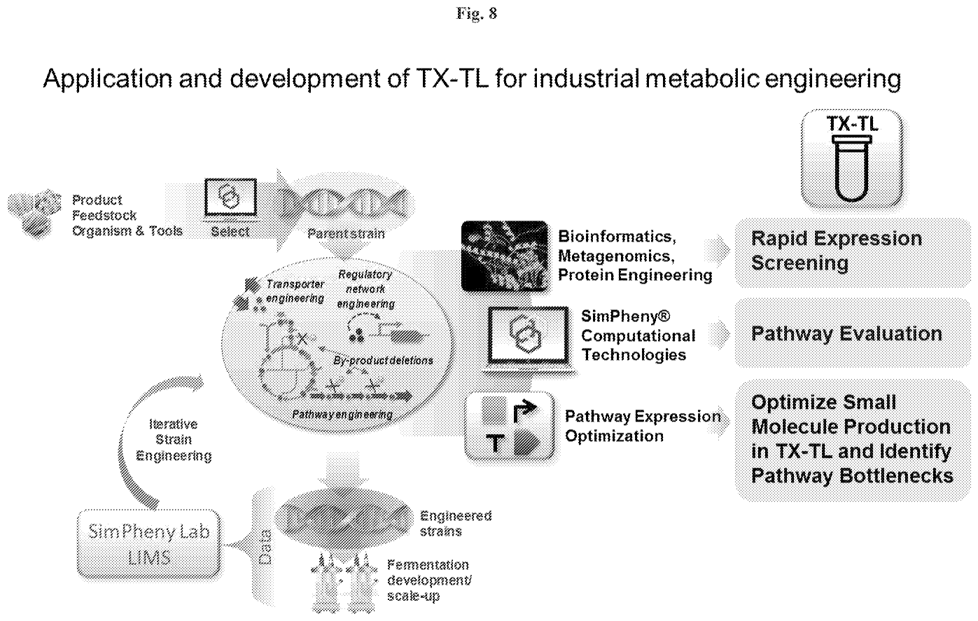

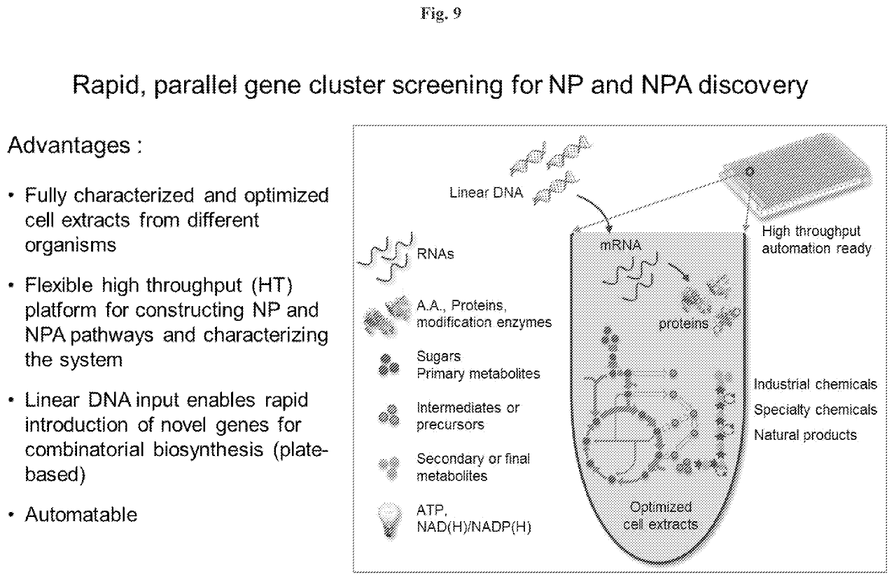

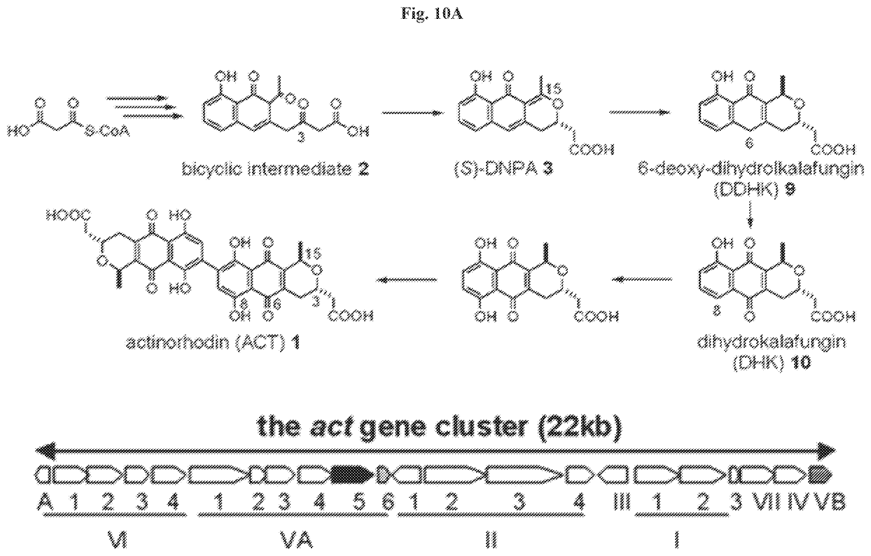

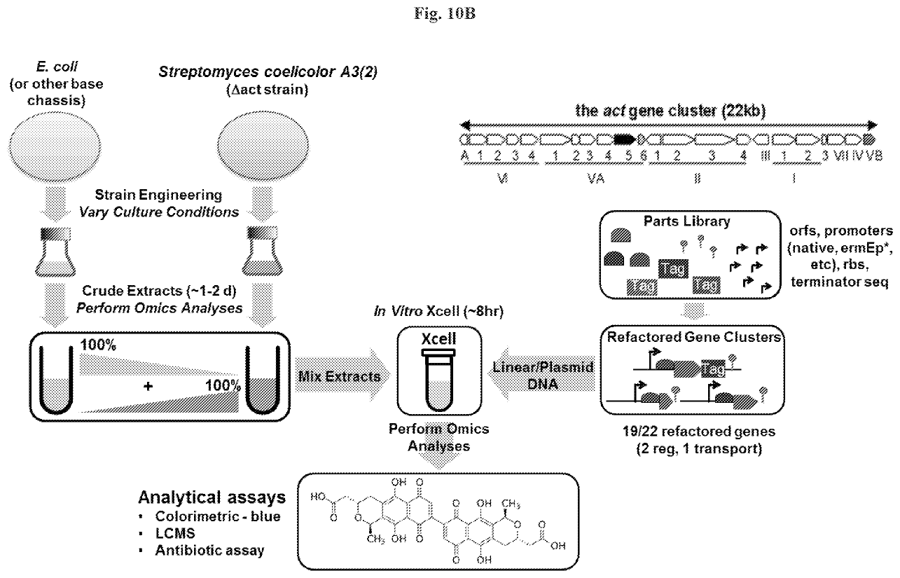

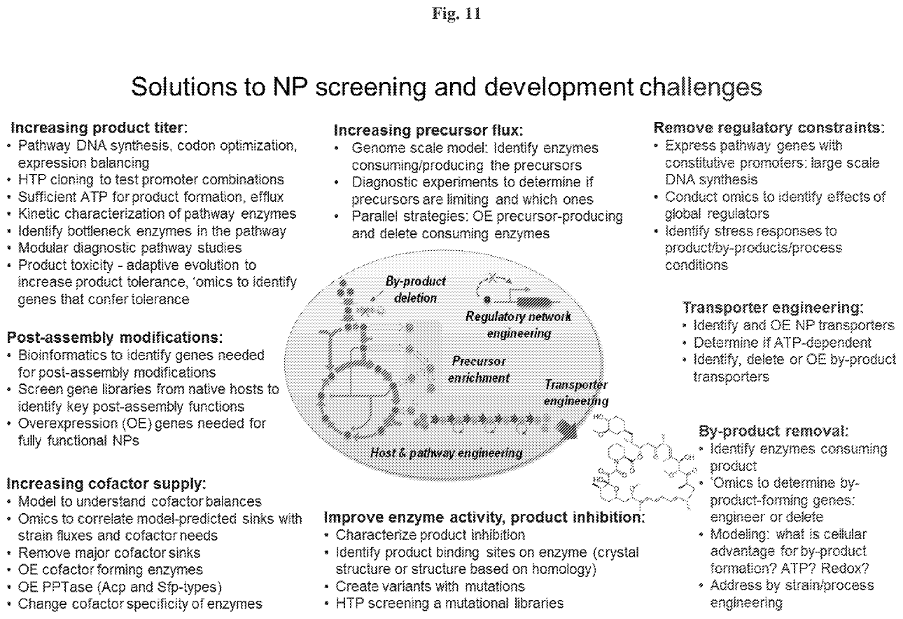

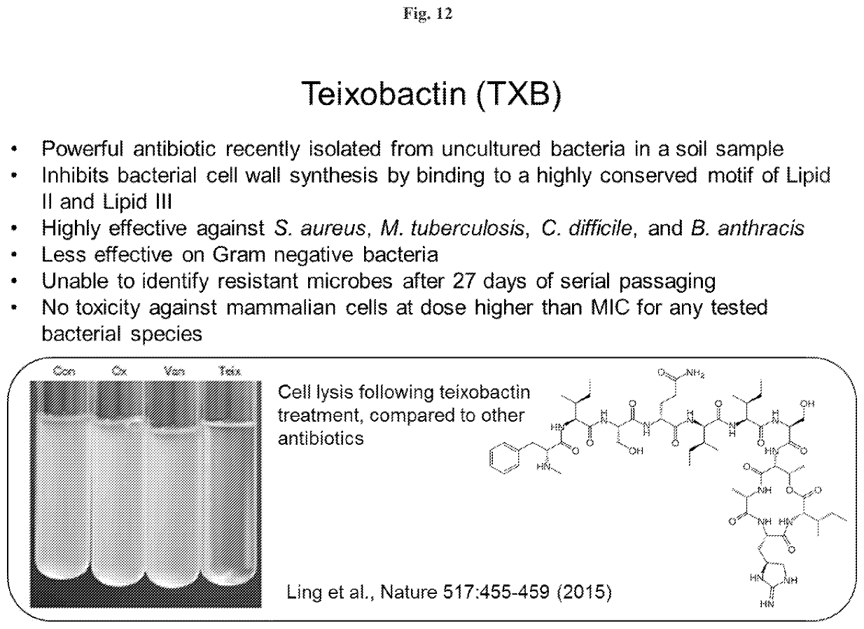

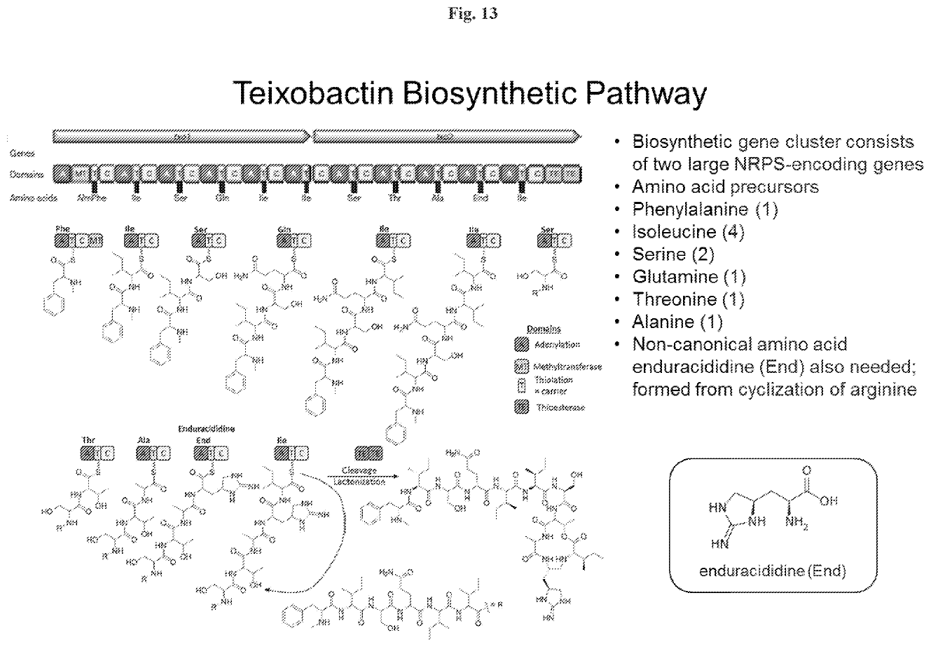

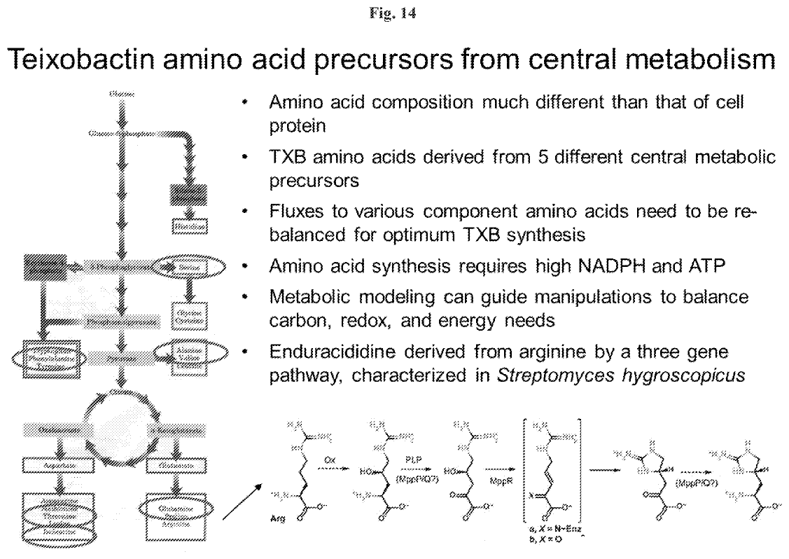

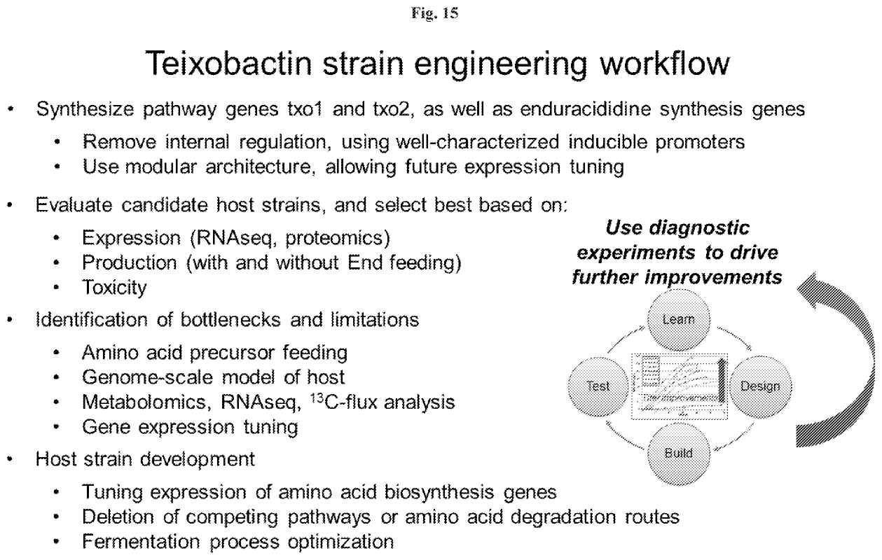

Genetic architecture of DEBS. DEBS (‘6-deoxyerythronolide B synthase”) catalyzes formation of 6-deoxyerythronolide B (1) from decarboxylative condensations between one propionyl-CoA priming unit and six methylmalonyl-CoA extender units. For β-carbon processing, modules 1, 2, 5, and 6 contain KR domains, module 4 contains the complete KR, DH, and ER set, and module 3 lacks any functional β-carbon-modifying domains; see FIG. 7. The loading segment consists of priming AT and ACP domains, and a thioesterase (TE) catalyzes the release and cyclization of the polyketide chain. For this work, restriction endonuclease sites were engineered around AT and KR domains in modules 2, 5, and 6. (B, BamHI; P, PstI; X, XbaI; KS, ketosynthase).

Exemplary TX/TL Application: Using automated Type II restriction enzyme cloning, synthetic variants for each of the DEBS genes are constructed using alternate AT and KR domains in each module. Considering 3 variants of each module (wild-type and one each of AT and KR domain changes), each of the genes consisting of 2 modules can have 9 variations. Linear DNA for these variants of each of the 3 DEBS genes are constructed, and then combined in vitro to make 729 different combinations. The combined DNA are added to a TX/TL mixture containing all the other genes required for erythromycin synthesis, and the reaction carried out in 10 uL aliquots in 96-well plates. After the reaction, the mixtures are transferred to cultures of the target microbe, to determine killing efficacy compared to that of the mixture with all wild-type genes.

### Example 4: Mutagenesis of Tailoring Enzymes

Provided herein are processes for combinatorial biosynthesis, including the modification (engineering) of enzymes, to generate products, e.g., novel natural products, and exemplary processes are described in this Example:

Mithramycin (MTM, also known as aureolic acid) is an aureolic acid-type polyketide produced by various soil bacteria of the genus Streptomyces, including Streptomyces argillaceus (ATCC 12956). Three novel MTM derivatives, namely mithramycin SK (MTM-SK), demycarosyl-mithramycin SK (demyc-MTM-SK), and mithramycin SA (MTM-SA), were generated and by insertional inactivation of the gene mtmW from the MTM producer Streptomyces argillaceus, which encodes a reductase involved in ketoreduction of the C-3 side chain of MTM. The structures of the new MTM derivatives were elucidated by NMR spectroscopy and mass spectrometry. One of these compounds, MTM-SK, was tested in vitro against a variety of human cancer cell lines, as well as in an in vitro toxicity assay, and showed an improved therapeutic index, in comparison to the parent drug, MTM. Protocols are described in e.g., Remsing et al., J Am Chem Soc. 2003 May 14; 125(19): 5745-5753.

Exemplary TX/TL Application: The TX/TL reaction are prepared using all S. argillaceus genes required for MTM synthesis, except for the mtmW gene which will be left out. The reactions are carried out in 10 uL aliquots, and the range of products made are tested by mass-spectrometry, in which the MTM peak should be replaced by a different peak due to the lack of ketoreduction. The structures are confirmed by NMR.

### Example 5: Generation of Novel Natural Products by Alteration of Precursors

Provided herein are processes for combinatorial biosynthesis, including the alternation, modification (engineering) of precursors, to generate products, e.g., novel natural products, and exemplary processes are described in this Example: Fluoroacetate is formed by Streptomyces cattleya by incorporating fluoride and can be activated to form fluoroacetyl-CoA. In this work, fluoromalonyl-CoA was generated either by carboxylation of fluoroacetyl-CoA or from fluoro-malonate. It was demonstrated that the thioesterase domain of deoxyerythronolide B synthase could use the fluorinated monomer also. This demonstration of activity with the modified precursor led to further experiments where pathways were constructed involving two polyketide synthase systems and it was shown that fluoroacetate could be used to incorporate fluorine into the PK backbone in vitro. It could also be inserted site-selectively to replace an H atom and introduced into polyketides in vivo. Protocols are described in e.g., Walker et al., Science, 2013, 341, 1089-93.

Exemplary TX/TL Application: A purified CoA transferase is used in an in vitro reaction with fluoroacetate to synthesize a stock of fluoroacetyl-CoA. The deoxyerythronolide B gene cluster is refactored under strong promoters, and TX/TL would be conducted using the standard TX/TL mix and these synthetic genes. Various ratios of acetyl-CoA and fluoroacetyl-CoA are added to the system along with other required cofactors and precursors. Mass spectrometry and NMR are used to confirm the locations and degree of fluoroacetate incorporation into the polyketide product.

### Example 6. Genetic Changes for Combinatorial Biosynthesis of Lipopeptides: Deletion of an Amino Acid Modifying Enzyme

Provided herein are processes for combinatorial biosynthesis of products such as lipopeptides, including the deletion of an amino acid modifying enzyme, to generate products, e.g., novel natural products, and exemplary processes are described in this Example:

Daptomycin is a cyclic lipodepsipeptide antibiotic synthesized by a non-ribosomal peptide synthesis (NRPS) mechanism in Streptomyces roseosporus. It consists of 13 amino acids, some of which are non-canonical, coupled to a long-chain fatty acid tail. The daptomycin gene cluster consists of 3 genes encoding NRPS, 3 genes responsible for fatty acid synthesis and ligation, 2 genes encoding amino acid modification enzymes, and 6 genes encoding regulatory, transport, and resistance mechanisms. Due to homology between daptomycin and related antibiotics from other organisms, it is hypothesized that modifications to the gene cluster could lead to structural changes in the molecule that result in improved and/or altered antimicrobial activity. Protocols are described in e.g., Nguyen et al. 2006, PNAS 103:17462-17467; Baltz 2014, ACS Syn. Biol. 3:748-758.

One example is the deletion of dptI (alpha-KG methyltransferase) which substitutes the 3-methylglutamate at position 12 with glutamate. The resulting cluster produces a molecule with 50% yield compared to wild-type, with reduced antimicrobial activity against S. aureus in the absence of surfactant but with better activity (lower MIC) in the presence of surfactant than the wild-type.

Exemplary TX/TL Application: The TX/TL reactions are prepared using all S. aureus genes required for daptomycin synthesis, except for the dptI gene which will be left out. The reactions are carried out in 100 uL aliquots, and the range of products made will be tested by mass-spectrometry, in which the daptomycin peak should be replaced by a different peak due to the substitution of glutamate for 3-methylglutamate at position 12. The structures are confirmed by NMR.

### Example 7. Genetic Changes for Combinatorial Biosynthesis of Lipopeptides: Domain Substitution in Nonribosomal Peptide Synthetases (NRPS)

Provided herein are processes for combinatorial biosynthesis of products such as lipopeptides, including domain substitutions, e.g., in NRPS enzymes, to generate products, e.g., novel natural products, and exemplary processes are described in this Example:

NRPS enzymes, as described above, comprise a series of modules, each responsible for the addition of an amino acid or fatty acid to the peptide chain. Each module consists of 3 or 4 domains: coupling amino acids or fatty acids together (C), binding and activation of amino acids (A), peptidyl carrier protein domains for tethering the amino acids to the growing chain (T), and optionally an (E) domain for interconverting L- and D-amino acids, (M) domain for amino acid methylation, and thioesterase (Te) for cyclization and release of the completed molecule.

Replacement of a module consisting of C-A-T domains has been shown to modify the amino acid incorporated at a particular position. For example, the C-A-T at position 11 can be replaced with the position 8 module, resulting in D-Ala substitution for D-Ser, and the opposite can be done to change position 8 from D-Ala to D-Ser. Both result in lower MIC against S. aureus in the presence of surfactant compared to the native molecule. Alternatively, different domain substitutions result in D-Asn substitution at either position. These have higher MIC against S. aureus, but could have improved activity against other microbes. Protocols are described in e.g., Nguyen et al. 2006, PNAS 103:17462-17467; Baltz 2014, ACS Syn. Biol. 3:748-758.

Exemplary TX/TL Application: Synthetic variants for each of the NRPS genes for daptomycin synthesis will be constructed, with all of the variants at positions 8 and 11 described above: modules for D-Ser, D-ala, and D-Asn at each of the positions. The combined DNA will be added to a TX/TL mixture containing all the other genes required for daptomycin synthesis, and the reaction carried out in 100 uL aliquots. The structures of the resulting products will be determined by NMR, to validate each of the substitutions.

### Example 8. Exemplary TX-TL Materials and Methods

Provided herein are processes for combinatorial biosynthesis of products such as, e.g., natural products (NPs), NP analogs (NPAs) and secondary metabolite analogs, and exemplary processes are described in this Example: Genes of interest were cloned into a plasmid suitable for expression in E. coli, plasmid pZS*13S obtained from R. Lutz (Expressys, Germany) and are based on the pZ Expression System (Lutz, R. & Bujard, H. Independent and tight regulation of transcriptional units in Escherichia coli via the LacR/O, the TetR/O and AraC/I1-I2 regulatory elements. Nucleic Acids Res. 25, 1203-1210 (1997)).

Cell-Free Expression Preparation and Execution:

Preparation of the cell-free TX-TL expression system was prepared e.g., as described in ACS Synth. Biol. 10.1021/sb400131a; in one exemplary embodiment the final extract has conditions: 8.9-9.9 mg/mL protein, 4-10.5 mM Mg-glutamate, 4-160 mM K-glutamate, 0.33-5 mM DTT, 1.5 mM each amino acid except leucine, 1.25 mM leucine, 50 mM HEPES, 1.5 mM ATP and GTP, 0.9 mM CTP and UTP,0.2 mg/mL tRNA, 0.26 mM CoA, 0.33 mM NAD, 0.75 mM cAMP, 0.068 mM folinic acid, 1 mM spermidine, 30 mM 3-PGA, 35 mM maltodextrin, 3.5 μM GamS, 30 mM pyruvate, 1 mM acetyl-coA and 2% PEG-8000 (Sun et al., (2013) Linear DNA for rapid prototyping of synthetic biological circuits in an Escherichia coli based TX-TL cell-free system.

TX-TL reactions were conducted in a volume of 10 μL in a 96-well plate (Nunc) at 29° C., using a three tube system: extract, buffer, and DNA. When possible, inducers such as IPTG or purified proteins such as gamS were added to a mix of extract and buffer to ensure uniform distribution. End point measurements are after 8 h of expression at 29° C.

Analytical Methods

3-HB, 4-HB and GBL can be separated by, for example, HPLC using a Spherisorb 5 ODS1 column and a mobile phase of 70% 10 mM phosphate buffer (pH=7) and 30% methanol, and detected using a UV detector at 215 nm (Hennessy et al. 2004, J. Forensic Sci. 46(6):1-9). 1,3-BDO and 1,4-BDO can be detected by gas chromatography or by HPLC and refractive index detector using an Aminex HPX-87H column and a mobile phase of 0.5 mM sulfuric acid; e.g., using protocols as described in e.g., Gonzalez-Pajuelo et al., Met. Eng. 7:329-336 (2005).

### Example 9. Use of TX-TL for Rapid In Vitro Generation of Violacein Analogs

Provided herein are processes for combinatorial biosynthesis of natural products such as, e.g., violacein and analogs thereof, and exemplary processes are described in this Example:

Processes as provided herein are used to rapidly generate analogs of violacein, a violet pigment with antitumor and antimicrobial properties.

A TX-TL cell-free protein synthesis method can be used in the production of the “parent” molecule violacein, e.g., an exemplary TX-TL coupled transcription/translation (TX-TL) systems process as provided herein, or a process as described by e.g., Sun et al. Protocols for Implementing an Escherichia coli Based TX-TL Cell-Free Expression System for Synthetic Biology. Journal of Visualized Experiments, 2013.

Exemplary TX-TL coupled transcription/translation (TX-TL) systems as provided herein are used to rapidly generate analogs of violacein: Genes for the violacein pathway VioABCDE from C. violaceum (GenBank ID: AB032799) are codon optimized by DNA 2.0™ and cloned into a plasmid suitable for expression in E. coli plasmid pET25b+ obtained from Novagen.

Tryptophan analogs (Sigma-Aldrich) were added to the TX-TL reactions to produce analogs of violacein.

Using E. coli (BL21 Star, Thermo Fisher) cellular extracts, individual plasmids containing the VioA, VioB, VioC, VioD, VioE genes are expressed in 10 μl reactions for 8 hours at 22° C. and were then pooled together along with 1 mM NADH, 1 mM NADPH, and 3 mM L-Tryptophan and incubated for an additional 20 hours at 22° C. The samples are spun down, e.g., at 21,000 RCF for 10 minutes, to extract the insoluble violacein analogs for analytical measurement.

Analysis of violacein, its pathway intermediates and analogs can be performed by LCMS. LCMS system can comprise e.g., an EXACTIVE™ high resolution mass spectrometer, ACCELA™ quaternary pump and THERMOPAL™ (ThermoPal™) autosampler (Thermofisher). Reversed phase chromatography on HYPERSIL GOLD™ 100×3 mm, 1.9 uM column can be used. Eluents: water with 0.1% formic acid and acetonitrile with 0.1% formic acid; acetonitrile gradient 20% to 95% over 8 minutes (m) at flow rate 300 uL/min. Data is acquired in positive ionization mode using e.g., XCALIBUR™ software.

Variations of this exemplary TX/TL process can be used to rapidly generate analogs of any natural product, including analogs of violacein.

### REFERENCES EXAMPLE 1

1. Sun Z Z et al. ACS Synth Biol. 2014 Jun. 20; 3(6):387-97.
2. Stephanie Culler, SEED2015 conference talk: “The development of platform-based technologies for the optimization of sustainably produced chemicals”, Jun. 11, 2015
3. http://www.jove.com/video/50762/protocols-for-implementing-an-Escherichia-coli-based-tx-tl-cell-free
4. Jewett et al. Mol Syst Biol. 2008; 4: 220
5. Hodgman C E and Mike Jewett: Metab Eng. 2012 May; 14(3):261-9.
6. Carlson et al. Biotechnol Adv. 2012 September-October; 30(5):1185-94.
7. Caschera and Noireaux Biochimie, 2014 April; 99:162-8.
8. Caschera and Noireaux, Metab Eng. 2015 January; 27:29-37.
9. Weber T, et al. Trends Biotechnol. 2015 January; 33(1):15-26.
10. Berdy J, J Antibiot (Tokyo). 2012 August; 65(8):385-95.
11. Weber et al. Nucleic Acids Res. 2015 Jul. 1; 43 (W1): W237-43.
12. Frasch et al. Curr Opin Biotechnol. 2013 December; 24(6):1144-50.
13. Nett M. et al. Nat Prod Rep. 2009 November; 26(11):1362-84.
14. Shin J, Noireaux V. J Biol Eng. 2010 Jul. 1; 4:9.
15. Briegel K J, et al. Genetics. 2006 January; 172(1):7-15.
16. Lee K A and Jung K H. J Nanosci Nanotechnol. 2011 May; 11(5):4261-4.
17. Na et al. Nat Biotechnol. 2013 February; 31(2):170-4.
18. Hodgman and Jewett et al. N Biotechnol. 2014 Sep. 25; 31(5):499-505.
19. Zubay G, Annu Rev Genet. 1973; 7:267-87.
20. Kim et al. J Bacteriol. 2008 June; 190(11): 3851-3858.
21. Wang et al. Appl. Environ. Microbiol. July 2013 vol. 79 no. 14 4484-4492.
22. Sohoni et al. PLoS One. 2014 Jun. 25; 9 (6): e99701.
23. Seghezzi et al. Appl Microbiol Biotechnol. 2011 April; 90(2):615-23.
24. Gomez-Escribano and Bibb et al. Microb Biotechnol. 2011 March; 4(2):207-15.
25. Zhou et al. FEMS Microbiol Lett. 2012 August; 333(2):169-79
26. Hu and Ochi et al. Appl Environ Microbiol. 2001 April; 67(4):1885-92.
27. Chiang et al. Curr Opin Chem Biol. 2011 February; 15(1):137-43.
28. Cobb et al. ACS Synth Biol. 2015 Jun. 19; 4(6):723-8.
29. Herrmann et al. Appl. Environ. Microbiol. March 2012 vol. 78 no. 6 1804-1812.
30. Christian et al. J Nat Prod. 2005 November; 68(11):1592-7.
31. Bode et al. Chembiochem. 2002 Jul. 2; 3(7):619-27.
32. Craney et al. Chem Biol. 2012 Aug. 24; 19(8):1020-7.
33. Antoraz et al. Front Microbiol. 2015; 6: 461.

A number of embodiments of the invention have been described. Nevertheless, it will be understood that various modifications may be made without departing from the spirit and scope of the invention. Accordingly, other embodiments are within the scope of the following claims.

1-56. (canceled)

57. A product of manufacture comprising a mixture of at least two extracts,
wherein the at least two extracts comprise:
(a) at least two different cytoplasmic extracts from at least two different cells; or
(b) at least one cytoplasmic and one nuclear extract from at least two different cells,
wherein at least one of the at least two different cells is an E. coli cell comprising modifications,
and wherein the E. coli cell modifications comprise:
(i) overexpression of an RNA polymerase, optionally a T7 or T3 RNA polymerase,
(ii) overexpression of an E. coli sigma factor,
(iii) addition of a mutated promoter of a lacI gene, called lacIQ, thereby leading to higher levels of expression of LacI, wherein optionally the comprises lacIq,
(iv) expression of rare tRNAs, and
(v) expression of gamS, thereby improving transcription from linear DNA, and the mixture is capable of in vitro transcription, translation and/or coupled transcription and translation.

58. The product of manufacture of claim 57, wherein: the at least two different cells are from different kingdoms, phyla, classes, orders, families, genera or species.

59. The product of manufacture of claim 57, wherein between about 50% and 99.9% of the mixture is from one of the at least two extracts.

60. The product of manufacture of claim 57, wherein at least one of the two different cells is a Streptomyces or an Actinomyces, and optionally the Actinomyces is an Amycolatopsis, a Saccharopolyspora, or a Micromonospora.

61. The product of manufacture of claim 57, further comprising additional ingredients, compositions or compounds, reagents, ions or elements, buffers and/or solutions,
wherein optionally the additional ingredients, compositions or compounds, reagents, ions or elements, buffers and/or solutions are mixed into the cytoplasmic extract, or the cytoplasmic and nuclear extracts mixture,
wherein optionally the additional ingredients, compositions or compounds, reagents, buffers and/or solutions comprise: nucleosides or nucleotides; lipids or fatty acids; carbohydrates, polysaccharides or sugars; nucleic acids or oligonucleotides; one or more enzymes, co-enzymes or enzyme co-factors; one or more amino acids; polycationic aliphatic amines or spermidine; a folinic acid, a 5-formyltetrahydrofolate or a leucovorin; a vitamin; a polyether or a polyethylene glycol (polyethylene oxide (PEO) or polyoxyethylene (POE)); a small-molecule redox reagent, an isopropyl β-D-1-thiogalactopyranoside (IPTG), a dithioerythritol (DTE) or a dithiothreitol (DTT); a glutamate or a glutamic acid; an alpha-keto amino acid or a pyruvic acid; a regulator or activator of transcription or translation, or any combination thereof;
wherein optionally the carbohydrate, polysaccharide or sugar comprises a maltodextrin, maltose, glucose and/or a hexametaphosphate (HMP);
and optionally the co-enzyme or co-factor comprises an acyl-CoA precursor (optionally acetyl-CoA, malonyl-CoA, ethylmalonyl-CoA, methylmalonyl-CoA, isobutyryl-CoA, or propionyl-CoA), a nicotinamide adenine dinucleotide (NAD) or an NADH, a nicotinamide adenine dinucleotide phosphate (NADP) or an NADPH, a fluoromalonyl-CoA (F-CoA), or a S-Adenosyl methionine (SAM);
and optionally the nucleosides or nucleotides comprise ATP, GTP, CTP, UTP or any combination thereof, and optionally the nucleic acids or oligonucleotides comprise transfer RNA (tRNA), small inhibitory RNA (siRNA), translational riboregulators or riboswitches,
and wherein optionally the amino acid comprises non-natural amino acid, optionally comprising (amino acids introduced using an expanded genetic code, and optionally the ions or elements comprise an inorganic phosphate, a phosphonate, a phosphonic acid or a phosphonate salt,
and optionally the one or more enzymes comprise an enzyme for modification of a product, a small molecule, a natural product, a secondary metabolite or a natural product analog (NPA), a protein, a lipid or fatty acid, a polysaccharide or a nucleic acid,
and optionally the enzyme modification comprises: lipidation, pegylation, glycosylation, adding hydrophobic groups, myristoylation, palmitoylation, isoprenylation, prenylation, lipoylation, adding a flavin moiety,
and optionally comprising addition of: a flavin adenine dinucleotide (FAD) an FADH₂, a flavin mononucleotide (FMN), or an FMNH₂), phospho-pantetheinylation, heme C addition, acylation, alkylation, butyrylation, carboxylation, malonylation, hydroxylation, adding a halide group, iodination, propionylation, S-glutathionylation, succinylation, glycation, adenylation, thiolation, or condensation,
and optionally the condensation comprises addition of: an amino acid to an amino acid, an amino acid to a fatty acid, an amino acid to a sugar, or a combination thereof,
and optionally the one or more enzymes comprise a CoA ligase, a phosphorylase, a glycosyl-transferase, a halogenase, a methyltransferase, a hydroxylase, or a lambda phage GamS enzyme,
and optionally used with a bacterial or an E. coli extract, optionally at a concentration of about 3.5 mM, a disulfide bond (Dsb) family enzyme (optionally DsbA), or a combination thereof;
or optionally the enzymes comprise one or more central metabolism enzyme (optionally tricarboxylic acid cycle (TCA, or Krebs cycle) enzymes, glycolysis enzymes or Pentose Phosphate Pathway enzymes),
and optionally the additional ingredients, compositions or compounds, reagents, ions, buffers and/or solutions comprise protease, DNase or RNase inhibitors, a buffer, an anti-oxidant, a rare earths, a vitamin, a salt, or a metal,
and optionally a trace metal, iron (Fe), zinc (Zn), Mg2+, Mn, vanadium and/or a halogen,
and optionally the additional ingredients, compositions or compounds, reagents, ions, buffers and/or solutions comprise a labeling agent (and optionally the labeling agent comprises a metabolic labeling agent), a detection or an affinity-tags, a fluorophore, reagents for biotinylation or biotin, a gold nanoparticle, an isotope or a radioactive isotope (optionally the isotope comprises a metabolic labeling isotope, a ¹³C-6-lysine, a ³H thymidine, a ³⁵S methionine, a ³²P orthophosphate, a ¹⁴C-labeled D-glucose); a photoreactive amino acid label (optionally the photoreactive amino acid label comprises a diazirine), a bioorthogonal labeling reagent (optionally the bioorthogonal labeling reagent comprises an azide, an alkyne, an aldehyde or a ketone), or an RNA polymerase,
optionally a T7 or T3 RNA polymerase,
optionally a purified polymerase, or a sigma factor,
optionally a Streptomyces sigma factor or a sigma 70, a sigma 54, sigma factors HrdB, 19, 24, 28, 32 and/or 38,
and optionally the additional ingredients, compositions or compounds, reagents, ions, buffers and/or solutions comprise an E. coli extract, an amino acid, HEPES, ATP, GTP, CTP and UTP, tRNA, CoA, NAD+, cAMP, folinic acid, spermidine, PEG-8000, Mg-glutamate, K-glutamate, DTT, NADPH, maltodextrin, IPTG, pyruvate or NADH,
wherein optionally the nucleic acids comprise:
an isolated or a synthetic nucleic acid comprising or encoding:
an enzyme-encoding natural-product (NP)- or natural product analog (NPA)-synthesizing operon; or a biosynthetic gene cluster,
optionally a biosynthetic gene cluster comprising coding sequence for enzymes needed in the synthesis of a natural product (NP), NPA, or a secondary metabolite;
a plurality of enzyme-encoding nucleic acids;
a plurality of enzyme-encoding nucleic acids for at least two, several or all of the steps in the synthesis of a product or chemical or molecule,
wherein optionally the product or chemical is a natural product (NP) or a natural product analog (NPA),
wherein optionally the chemical, the natural product (NP), the NPA, or secondary metabolite is a violacein, a butadiene, a propylene, a 1,4-butanediol, a 1,3-butanediol, a crotyl alcohol, a methyl vinyl carbinol, an isopropanol, an ethylene glycol, a terephthalic acid, an adipic acid, a hexamethylenediamine (NMDA), a caprolactam, a cyclohexanone, a aniline, a Methyl Ethyl Ketone (MEK), a fatty alcohol, an acrylic acid, an acrylate ester, a methyl methacrylate, a lipid, a carbohydrate, a beta-lactam, a polyketide, a macrolide, a macrolide having a 14-, 15- or 16-membered macrocyclic lactone ring, a trans-AT type I PKS, a Type II PKS, or a Type III PKS, a heterocyst glycolipid PKS-like, a cyclic peptide, a bottromycin or an antibiotic,
and optionally the chemical, the natural product (NP), the NPA, or secondary metabolite is a terpenoid, a steroid, an alkaloid, a fatty acid, a nonribosomal polypeptide (“NRP”; also referred to as “nonribosomal peptide”), an enzyme cofactor, an aminocoumarin, a melanin, an aminoglycosides/aminocyclitol, a microcin, an aryl polyene, a microviridin, a bacteriocin, a nucleoside, an oligosaccharide, a butyrolactone, a phenazine, a phosphoglycolipid, a cyanobactin, a phosphonate, a (dialkyl)resorcinol, a polyunsaturated fatty acid, an ectoine, a furan, a lycocin, a Head-to-tail cyclized peptide, a proteusin, a homoserine lactone, a sactipeptide, an indole, a siderophore, a ladderane lipid, a terpene, a lantipeptide, a thiopeptide, a linear azol(in)e-containing peptides (LAPs), a lasso peptide, a linaridin,
and optionally the isolated or the synthetic nucleic acid are in a linear or a circular form,
and optionally the isolated or the synthetic nucleic acid is contained in a circular or a linearized plasmid, vector or phage DNA,
and optionally the isolated or the synthetic nucleic acid comprises enzyme coding sequences operably linked to a homologous or a heterologous transcriptional regulatory sequence,
optionally a transcriptional regulatory sequence comprises a promoter, an enhancer, or a terminator of transcription,
and optionally the isolated or the synthetic nucleic acid comprises has at least about 100, 200, 300, 400 or 500 or more base pair ends upstream of the promoter and/or downstream of the terminator,
and optionally the promoter comprises a native promoter, and optionally the native promoter comprises a promoter used in an organism that is a source of the mixed extract, or a synthetic promoter,
and optionally promoters operably linked to nucleic acids comprise a combination of promoters from all or several of the organisms that are the source of the mixed extract (optionally the combination of promoters comprises an E. coli promoter and a Streptomyces promoter,
and optionally the promoter is ermEp*, and optionally the promoter is a heterologous promoter from Saccharopolyspora erythraea active in E. coli, SF14p, or kasOp*,
and optionally all, or a subset, of the enzyme-encoding nucleic acid of the enzyme-encoding natural-product (NP), NPA, or a synthesizing operon or biosynthetic gene cluster are contained on separate linear nucleic acids, optionally in equimolar concentrations in the mixed cytoplasmic or nuclear extract,
and optionally, each separate linear nucleic acid comprises one, two, three, 4, 5, 6, 7, 8, 9, or 10 or more genes or enzyme-encoding sequences, and optionally the linear nucleic acid is present in a concentration of about 1.0 nanomolar mN, 5 nM, 10 nM, 15 nM, 20 nM, 25 nM, 30 nM, 35 nM, 40 nM, 45 nM or 50 nM or more or between about 1 nM and 100 nM,
and optionally the enzyme-encoding nucleic acids, the linear nucleic acid or all, or a subset, of the enzyme-encoding nucleic acid of the enzyme-encoding natural-product synthesizing operon or biosynthetic gene cluster are immobilized, optionally immobilized on a bead or a chip,
and optionally the enzyme encoded by the nucleic acid are between about 10 and 100 kDa, or about 10 kDa, 20 kDa, 30 kDa, 40 kDa, 50 kDa, 60 kDa, 70 kDa, 80 kDa, 90 kDa, or 100 kDa, 110 kDa, 120 kDa, 130 kDa, 140 kDa, 150 kDa, or more, and optionally the isolated or the synthetic nucleic acid comprises:
(i) a genome, a gene or a DNA from a source other than the cell used for the extract, or an exogenous nucleic acid that has been engineered or mutated, optionally engineered or mutated in a protein coding region or in a non-coding region,
(ii) a genome, a gene or a DNA from a cell used for the extract, or an endogenous nucleic acid that has been engineered or mutated, optionally engineered or mutated in a protein coding region or in a non-coding region,
(iii) a genome, a gene or a DNA from one, both or several of the organisms used as a source for the extract, or
(iv) any or all of (i) to (iii),
wherein optionally the enzyme-encoding natural-product (NP), natural product analog (NPA), or synthesizing operon; the biosynthetic gene cluster; or the plurality of enzyme-encoding nucleic acids for at least two, several or all of the steps in the synthesis of a natural product (NP), natural product analog (NPA) or secondary metabolite or a chemical or molecule:
(i) comprise one or several cryptic, or phenotypically silent, genes, optionally identified by a software or sequence analysis of a genome,
(ii) are genetically modified, optionally modified for optimization of transcription, translation and/or function of an encoded protein,
and optionally RNA encoding sequence are optimized, and optionally the enzyme-encoding natural-product (NP) or natural product analog (NPA)-synthesizing operon; the biosynthetic gene cluster; or the plurality of enzyme-encoding nucleic acids for at least two, several or all of the steps in the synthesis of a natural product (NP), natural product analog (NPA) or secondary metabolite or a chemical or molecule are identified by methods comprising use of: a genomic or biosynthetic search engine, or, an Integrated Microbial Genomes (IMG)-ABC system (DOE Joint Genome Institute (JGI)).

62. The product of manufacture of claim 57, wherein the at least two cell extracts further comprise at least one extract from: a different prokaryotic cell or a eukaryotic cell; or, a different bacterial cell, a fungal cell, a yeast cell, an algae cell, an Archaeal cell, an insect cell, a plant cell, a mammalian cell or a human cell.

63. The product of manufacture of claim 57, wherein the mixture further comprises:
(i) an undiluted liquid isolate from at least one of the at least two different cells;
(ii) a diluted liquid preparation from at least one of the at least two different cells,
wherein optionally the cytoplasmic or combined cytoplasmic and nuclear extracts is diluted with a saline or a buffer;
(iii) an undiluted liquid preparation from at least one of the at least two different cells;
(iv) a lyophilized preparation of the mixture of at least two cytoplasmic extracts or combined cytoplasmic and nuclear extracts from at least two different cells;
(v) at least one cytoplasmic extract from a prokaryotic cell or a bacterial cell and at least one nuclear extract from a eukaryotic cell;
(vi) at least one cytoplasmic extract from a prokaryotic cell and at least one nuclear extract from a mammalian cell;
(vii) at least one cytoplasmic extract from a bacterial cell and at least one nuclear extract from an insect, a plant, a fungal or a yeast cell;
(viii) cytoplasmic extracts from at least two different bacterial cells;
(ix) at least one cytoplasmic or nuclear extract from or at least one cytoplasmic or nuclear extract obtained from:
(1) a prokaryote, a bacteria, an Archaea, a eukaryote, a fungi, a plant, an animal, or a human,
(2) a bacterial isolate from an environmental source or sample,
(3) a Saccharomyces cerevisiae or a yeast, a Aspergillus or fungus, optionally A. oryzae, A. nidulans, a plant or plant product, optionally a wheat germ, P. somniferum S. lycopersicon, M. esculenta, L. japonicas, A. thaliana, Zea mays, or Avena spp,
(4) an Escherichia or a Escherichia coli (E. coli); an Actinomyces or a Streptomyces or an Actinobacteria, a Micromonospora; an Ascomycota, Basidiomycota, or a Saccharomycetales; a Penicillium or a Trichocomaceae; a Spodoptera, a Spodoptera frugiperda, a Trichoplusia or a Trichoplusia ni; a Poaceae, or a Triticum;
(5) an insect cell, optionally Sf9,
(6) a rabbit reticulocyte, Chinese hamster ovary (CHO), Human embryonic kidney (HEK) or a HeLa cell, or a cultured human-derived cell; or
(7) Leishmania tarentolae, Myxobacteria, Phellinus, Ceratocystis virescens, Cronartium fusiforme, Paenibacillus polymyxa, Mycolatopsis rifamycinica, Clostridium botulinum, Streptomyces verticillus, Marine bacteria, Archaea, Thermococcus S557, Methanocaldococcus jannaschii, Penicillium chrysogenum, Cephalosporium acremonium, Pleurotus ostreatus, Tolypocladium inflatum, Claviceps spp., Aspergillus alliaceus, Taxus brevifolia, Cephalotaxus harringtonii, Artemisia annua, Galanthus spp., Conus magus, Conus magus, Ecteinascidia turbinate, Discodermia dissoluta, Erythropodium caribaeorum or Bugula neritina.

64. The product of manufacture of claim 59, wherein between about 51% and 99.5% of the liquid volume of the at least two extracts is from one of the at least two extracts.

65. The product of manufacture of claim 57, wherein one of the at least two cytoplasmic extracts comprises an extract from or comprises an extract obtained from a species selected from the group consisting of Streptomyces species S. coelicolor, S. albus, S. albus J1074, S. ambofaciens, S. ambofaciens BES2074, S. avermitilis, S. avermitilis SUKA17, S. coelicolor M1154, S. fradiae, S. roseosporus, S. toyocaensis, S. venezuelae, S. cinnamonensis, Streptomyces rapamycinicus, Streptomyces griseus, Streptomyces platensis, Streptomyces spheroides, Streptomyces rimosus, Streptomyces roseosporus and Streptomyces lividans;
and optionally the Amycolatopsis is Amycolatopsis mediterranei, or Amycolatopsis orientalis;
and optionally the Saccharopolyspora is Saccharopolyspora erythraea or Saccharopolyspora spinosa.

66. The product of manufacture of claim 57, wherein the cells from which the at least one cytoplasmic or the nuclear extract has been obtained, before isolation or harvesting of the extract, is:
an activated or a stimulated cell;
a cell exposed to chemical or a reagent in vitro;
a genetically altered cell;
a strain engineered cell, or a cell modified by genetic strain engineering methods, or a modification of cells from which the at least one cytoplasmic or nuclear extract has been derived; or,
a cultured cell.

67. The product of manufacture of claim 57, wherein the cells from which the at least one cytoplasmic or nuclear extract has been obtained:
(i) are free of cell wall, cell wall components, organelles or sub-cellular compartments; or
(ii) are supplemented with: an organelle or sub-cellular compartment, wherein optionally the organelle comprises a natural or a synthetic Golgi organelle, a mitochondria or a chloroplast; a synthetic or a designer organelle, a synthetic nano- or micro-compartment, a synthetic or a designer micelle or liposome; an NAD(P)H or ATP recycling system; a mitochondria or mitochondrial extract; or a chaperone protein or a chaperone complex or mbtH and its homologs, or a broad specificity 4′-Phosphopantetheine transferase or phosphoprotein phosphatase (PPTtase) including sfp and its homologs,
and optionally the natural or a synthetic Golgi organelle is for glycosylation,
and optionally the chaperone protein or the chaperone complex comprises any of Hsp60, Hsp70, Hsp90, Hsp100, DnaK-DnaJ-GrpE and/or GroEL-GroES.

68. The product of manufacture of claim 57, wherein in (a) one of the at least two cytoplasmic extracts comprises an extract from or comprises an extract obtained from an Actinomyces or a Streptomyces.

69. The product of manufacture of claim 57, wherein in (b) the nuclear extract comprises an extract from or comprises an extract obtained from a mammalian cell.

70. The product of manufacture of claim 57, wherein in (c) the cytoplasmic extract comprises an extract from or comprises an extract obtained from an Actinomyces or a Streptomyces, or
the nuclear extract comprises an extract from or comprises an extract obtained from a mammalian cell.

71. The product of manufacture of claim 57, wherein the mixture further comprises:
a biosynthetic gene cluster comprising a coding sequence for an enzyme needed in the synthesis of a natural product (NP), a natural product analog (NPA), or a secondary metabolite;
a plurality of enzyme-encoding nucleic acids; or
a plurality of enzyme-encoding nucleic acids for at least two, several or all of the steps in the synthesis of a product or chemical or molecule,
wherein optionally the product or chemical is a natural product (NP) or a natural product analog (NPA) or secondary metabolite analog.

72. The product of manufacture of claim 57, wherein the cells from which the at least one cytoplasmic or nuclear extract are obtained comprise components of a central metabolism,
wherein optionally the central mechanism comprises glycolysis, pentose phosphate pathway, TCA cycle and amino acid biosynthesis, lipid or fatty acid biosynthesis, oxidative phosphorylation and/or protein synthesis upregulated, activated or co-activated, or de-activated.

73. The product of manufacture of claim 72, wherein the cells from which the at least one cytoplasmic or nuclear extract has been obtained are cultured under different environmental or in vitro culture conditions,
and optionally the different environmental or in vitro culture conditions comprise turning on or off native enzymes, natural products or secondary metabolites, a natural product analog (NPA), or proteins, or for the extracts to have greater or fewer amounts of co-factors.

74. The product of manufacture of claim 73, wherein the natural products or secondary metabolites comprise polyketides of class I, II or III, a non-ribosomal peptide or a hybrid polyketide-non ribosomal peptide.

75. The product of manufacture of claim 72, wherein
the cells from which the at least one cytoplasmic or nuclear extract has been obtained are in mid-log phase, interphase, mitotic (M) phase or cytokinesis, or undergoing mitosis,
the strain engineering comprises ribosome and/or RNA polymerase engineering, and optionally comprising: adding or making rpoB and rpsL mutants, or components of ribosomal subunits, to enhance secondary metabolite production; mutations to the RNA polymerase machinery can be made to increase promoter binding affinity; deletion or overexpression of pathway or global regulators or activators and repressors; expressing mutant transcriptional regulators; and/or expressing or overexpressing ribosome recycling factor (RRF),
and optionally the strain engineering comprises epigenetic modifications, optionally comprising phosphorylation, acetylation, methylation, ubiquitination, ADP-ribosylation, and/or glycosylation,
and optionally the strain engineering comprises engineering in self-resistance, optionally by the upregulation or overexpression of a resistance gene, and optionally the resistance gene is drrABC, avtAB or actAB,
and optionally the strain engineering comprises genome-minimizing, optionally removing or disabling one, some, all or the majority of secondary metabolite biosynthetic gene clusters (SMBGCs),
and optionally the strain engineering comprises combinatorial knockdown of secondary metabolite pathways,
optionally by adding or expressing small RNAs targeting secondary metabolite biosynthesis,
and optionally the strain engineering comprises expressing tRNAs for rare codons,
optionally codons for AGA, AGG, AUA, CUA, GGA, CCC, and CGG,
and optionally the strain engineering comprises over-expressing one or more chaperones native to strain,
optionally a Streptomyces,
optionally a Streptomyces comprising over-expressing Hsp60, Hsp70, Hsp90, Hsp100, DnaK-DnaJ-GrpE and/or GroEL-GroES,
optionally a Streptomyces comprising over-expressing Hsp60, Hsp70, Hsp90, Hsp100, DnaK-DnaJ-GrpE and/or GroEL-GroES to improve overall protein production, and optionally the strain engineering comprises inactivating RNaseE,
optionally by mutation to enhance mRNA stability and consequently protein production,
and optionally the strain engineering comprises expressing or overexpressing Streptomyces antibiotic regulatory protein (SARP) for positive regulation of antibiotic production,
and optionally the strain engineering comprises expressing or overexpressing MbtH-like proteins for stimulating adenylation reactions,
and optionally the strain engineering comprises expressing or overexpressing phosphopantetheinyl transferases (PPTases) proteins for stimulating post-translational modification of an apo-acyl carrier protein (apo-ACP) to activate polyketide synthases, and optionally the strain engineering comprises NAD(P)H regeneration, optionally expressing or overexpressing trans-hydrogenases.

76. The product of manufacture of claim 57, wherein:
(a) step (b)(i) comprises overexpression of a T7 or T3 RNA polymerase, optionally a purified T7 or T3 RNA polymerase;
(b) step (b)(i) comprises overexpression of a sigma 70, sigma 54, sigma 19, sigma 24, sigma 28, sigma 32 or sigma 38 E. coli sigma factorC.
(c) step (b)(i) comprises adding purified sigma factors to the modified E. coli cell cytoplasm, or
(d) step (b)(iv) comprises transforming the modified E. coli cell with a plasmid that expresses rare codon tRNAs, wherein optionally the rare codon tRNAs comprise the rare codons AGA, AGG, AUA, CUA, GGA, CCC, and CGG, and optionally the rare tRNA genes are driven by a native promoter or by a synthetic promoter.
